(19) United States
(12) Patent Application Publication
(10) Pub. No.: US 2023/0159645 A1
(43) Pub. Date: May 25, 2023

(54) MULTISPECIFIC BINDING MOIETIES COMPRISING PD-1 AND TGF-BRII BINDING DOMAINS

(71) Applicants: MERUS N.V., Utrecht (NL); Incyte Corporation, Wilmington (DE)

(72) Inventors: Cecilia Anna Wilhelmina GEUIJEN, Utrecht (NL); Patrick Mayes, Wilmington, DE (US); Shaun M. Stewart, Wilmington, DE (US); Liang-Chuan Wang, Wilmington, DE (US)

(73) Assignees: MERUS N.V., Utrecht (NL); Incyte Corporation, Wilmington, DE (US)

(21) Appl. No.: 18/056,907

(22) Filed: Nov. 18, 2022

(30) Foreign Application Priority Data
Nov. 19, 2021 (NL) 2029844

(51) Int. Cl. C07K 16/28; A61P 35/00

(52) U.S. Cl. CPC C07K 16/2863; C07K 16/2818; A61P 35/00; C07K 2317/31; A61K 2039/505

(57) ABSTRACT

The present disclosure relates to a multispecific binding moiety comprising a PD-1 binding domain and a TGF-βRII binding domain, wherein the PD-1 binding domain blocks PD-1 mediated signaling and the TGF-βRII binding domain blocks TGF-βRII-mediated signaling. The present disclosure further relates to a pharmaceutical composition comprising such multispecific binding moiety, a method of treatment using such multispecific binding moiety, and a cell producing such multispecific binding moiety.

### REFERENCE TO AN ELECTRONIC SEQUENCE LISTING

The contents of the electronic sequence listing (AttachC_SeqListingST26-2189A.xml; Size: 149 kilobytes; and Date of Creation: Nov. 16, 2022) is herein incorporated by reference in its entirety.

### TECHNICAL FIELD

The present disclosure relates to the field of antibodies. In particular it relates to the field of therapeutic antibodies for the treatment of diseases involving aberrant cells. More particularly it relates to multispecific binding moieties comprising a binding domain that binds to PD-1 and a binding domain that binds to TGF-βRII.

### BACKGROUND

Although T lymphocytes are known for their role in immunosurveillance of tumors, cancer cells are capable of escaping immune control by induction of inhibitory immune pathways. Consequently, immune checkpoint blockade (ICB) in which antibodies are used to block inhibitory immune pathways, has emerged as a promising therapeutic option and has been demonstrated in preclinical and clinical studies to enhance and sustain endogenous immunity against certain cancers.

Programmed death 1 (PD-1) and programmed death ligand 1 (PD-L1) are components of an immunosuppressive network that dampens T cell activity in normal physiology but can be exploited by tumors to suppress T cell-mediated antitumor immune responses. Antibodies directed against PD-1 and PD-L1 have effected improvements in responses and survival for some patients suffering from several different cancers. However, despite promising clinical activity, only a minority of patients respond to anti-PD-1/PD-L1 therapies, with limited durability. Thus, there is an urgent need to develop new, safe and effective therapies to treat cancer.

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in downregulating the immune system by inhibiting the activation of T-cells, which in turn, when present on somatic cells, reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used to treat some types of cancer.

Monoclonal antibodies targeting PD-1 have been approved for the treatment of various malignancies. Response rate in melanoma patients treated with an anti-PD-1 antibody (pembrolizumab), for example, is 33% at 3 years despite 70-80% of patients initially responding (Ribas A. et al. Association of Pembrolizumab With Tumor Response and Survival Among Patients With Advanced Melanoma. JAMA. 2016 Apr. 19; 315(15):1600-9).

TGF-β signaling regulates a plethora of physiological and pathological processes including cell cycle arrest in epithelial and hematopoietic cells, control of mesenchymal cell proliferation and differentiation, wound healing, extracellular matrix production, immunosuppression and carcinogenesis (Massagué J. TGF-β signalling in context. Nat Rev Mol Cell Biol. 2012 October; 13(10):616-30). In addition, TGF-β signaling regulates numerous cancer cell functions, including cell cycle progression, apoptosis, adhesion and differentiation (Liu S et al, Signal Transduction and targeted Therapy, 2021). TGF-β exhibits a biphasic function such that in normal and premalignant cells, it predominantly has been reported to act as a tumor suppressor, whereas in tumor cells it permits growth promoting functions and epithelial-to-mesenchymal transition, which in turn permits tumor cell migration, invasion, intravasation and extravasation.

TGF-βRII is a member of the serine/threonine protein kinase family and the TGFB receptor subfamily. It is known under various synonyms, including TGFBR2, AAT3, FAA3, LDS1B, LDS2, LDS2B, MFS2, RIIC, TAAD2, TGFR-2, TGFbeta-RII, transforming growth factor beta receptor 2, TBR-ii, and TBRII. TGF-βRII forms a heterodimeric complex with another receptor protein, and binds TGF-β. This receptor/ligand complex phosphorylates proteins, which then enter the nucleus and regulate the transcription of a subset of genes related to cell proliferation.

Several inhibitors targeting the TGF-β pathway are in preclinical and clinical development and inhibit TGF-β at different levels; i.e. at the ligand, ligand-receptor level or intracellular level. The inhibitors include for instance TGF-β-neutralizing monoclonal antibodies, an anti-TGF-βRII antibody, soluble receptors, antibody ligand traps (e.g. anti-PD-L1-TGF-βRIIECD), antisense oligonucleotides to prevent TGF-β synthesis, a TGF-β2 antisense gene-modified allogeneic cancer cell vaccine, and small molecules targeting the kinase domain of TGF-βRI.

Targeting of the TGF-β pathway with a monospecific antibody is reported to show anti-tumor activity in vitro and in vivo; however, poor clinical outcomes persist with low efficacy and unacceptable toxicity, including major cytokine release syndrome (CRS). Combined targeting of the TGF-β pathway and immune checkpoint inhibition has been attempted with Bintrafusp alpha, an anti-PD-L1-TGFBRII bifunctional fusion protein, without demonstrating a strong clinical effect.

There remains a need for novel therapeutic interventions that selectively inhibit TGF-β signaling in activated tumor-specific PD-1-expressing T cells locally in the tumor microenvironment, in order to promote T cell tumor infiltration, and restore and sustain T cell antitumor effector activity. Such targeting will alleviate immunosuppressive pathways to potently promote CTL function and T cell memory for effective durable cancer elimination while minimizing toxicity associated with systemic TGF-β blockade.

### SUMMARY

One of the objects of the present disclosure is to provide a new pharmaceutical agent for the treatment of human disease, in particular for the treatment of cancer. This object is met by the provision of multispecific binding moieties, for example bispecific antibodies, that bind PD-1 and TGF-βRII. The PD-1 binding domain of the multispecific binding moieties aims at driving the specificity of the multispecific binding moiety to activated/exhausted effector T cells in tumors and tumor-draining lymph nodes, where the TGF-βRII binding domain can locally block TGF-β from binding to TGF-βRII, so as to reduce systemic toxicity of TGF-β inhibition on non-T cells. Further, the multispecific binding moieties alleviate both the PD1- and TGF-β-mediated immunosuppressive pathways to promote cytotoxic T lymphocyte activity in the tumor microenvironment.

The present disclosure provides a multispecific binding moiety comprising a PD-1 binding domain and a TGF-βRII binding domain, wherein the PD-1 binding domain blocks PD-1 mediated signaling and the TGF-βRII binding domain blocks TGF-βRII-mediated signaling.

The present disclosure also provides a multispecific binding moiety comprising a PD-1 binding domain and a TGF-βRII binding domain, wherein the PD-1 binding domain comprises a heavy chain variable region comprising CDR1, CDR2, and CDR3 sequences as described further herein.

The present disclosure also provides a multispecific binding moiety comprising a PD-1 binding domain and a TGF-βRII binding domain, wherein the TGF-βRII binding domain comprises a heavy chain variable region comprising CDR1, CDR2, and CDR3 sequences as described further herein.

The present disclosure further provides a pharmaceutical composition comprising an effective amount of the multispecific binding moiety as described herein.

The present disclosure further provides a multispecific binding moiety as described herein, and a pharmaceutical composition as described herein, for use in therapy.

The present disclosure further provides a multispecific binding moiety as described herein, and a pharmaceutical composition as described herein, for use in the treatment of cancer.

The present disclosure further provides a method for treating a disease, comprising administering an effective amount of a multispecific binding moiety as described herein, or a pharmaceutical composition as described herein, to an individual in need thereof.

The present disclosure further provides a method for treating cancer, comprising administering an effective amount of a multispecific binding moiety as described herein, or a pharmaceutical composition as described herein, to an individual in need thereof.

The present disclosure further provides a cell comprising a nucleic acid sequence encoding the heavy chain variable region of a PD-1 binding domain as described herein and a nucleic acid sequence encoding the heavy chain variable region of a TGF-βRII binding domain as described herein.

The present disclosure further provides a cell producing a multispecific binding moiety as described herein.

### DETAILED DESCRIPTION

In certain embodiments, the present disclosure provides a multispecific binding moiety comprising a PD-1 binding domain and a TGF-βRII binding domain, wherein the PD-1 binding domain blocks PD-1 mediated signaling and the TGF-βRII binding domain blocks TGF-βRII-mediated signaling.

As used herein, “blocks” or “blocking” means interfering or modifying the interaction between a ligand and a receptor, or causing a total or partial reduction of the signal transduction cascade. For the purposes of the present disclosure, in certain embodiments, potency in blocking ligand induced PD-1 signaling is determined by using a SHP recruitment assay as described in Example 2 or a NFAT reporter assay as described in Example 3. In certain embodiments, potency in blocking ligand induced PD-1 signaling is determined by using a SHP recruitment assay as described in Example 2. In certain embodiments, potency in blocking ligand induced PD-1 signaling is determined by using a NFAT reporter assay as described in Example 3. For the purposes of the present disclosure, in certain embodiments, potency in blocking ligand induced TGF-βRII signaling is determined by using a SMAD assay as described in Example 4 or 5. In certain embodiments, potency in blocking ligand induced TGF-βRII signaling is determined by using a SMAD assay as described in Example 4. In certain embodiments, potency in blocking ligand induced TGF-βRII signaling is determined by using a SMAD assay as described in Example 5.

In certain embodiments, a multispecific binding moiety of the present disclosure binds to human PD-1. In certain embodiments, the PD-1 binding domain of the multispecific binding moiety of the present disclosure blocks binding of PD-L1 to PD-1, for instance as measured in an assay as described in Example 2 or 3.

In certain embodiments, a multispecific binding moiety of the present disclosure binds to human TGF-βRII. Human TGF-βRII is a transmembrane protein of which there are different isoforms. The amino acid sequence of human TGF-βRII isoform A is provided as SEQ ID NO: 82; the amino acid sequence of the extracellular domain of human TGF-βRII isoform A is provided as SEQ ID NO: 83. Human TGF-isoform B is a splice variant encoding a longer isoform due to an insertion in the extracellular domain. The amino acid sequence of human TGF-βRII isoform B is provided as SEQ ID NO: 84; the amino acid sequence of the extracellular domain of isoform B of human TGF-βRII is as set forth in SEQ ID NO: 85.

A “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like VHH and VH), minibodies, scFab, scFv-zipper, nanobodies, DART molecules, TandAb, Fab-scFv, F(ab)′2, F(ab)′2-scFv2, and intrabodies.

In certain embodiments, the multispecific binding moiety is a multispecific antibody. A multispecific antibody according to the present disclosure is an antibody that comprises at least two binding domains which have specificity for at least two different targets or epitopes. In certain embodiments, a multispecific antibody of the present disclosure is a bispecific antibody. In certain embodiments, a multispecific antibody of the present disclosure may further comprise an Fc region or a part thereof. In certain embodiments, a multispecific binding moiety of the present disclosure is an IgG1 antibody. Constant regions of a binding moiety of the present disclosure may comprise one or more variations that modulate properties of the binding moiety other than its binding properties to the target antigens. For instance, the constant regions may comprise one or more variations that promote heterodimerization of the PD-1 and TGF-βRII heavy chains over homodimerization of two PD-1 heavy chains and/or two TGF-βRII heavy chains, and/or the constant regions may comprise one or more variations that reduce or improve effector function, in particular one or more variations that reduce effector function.

A “Fab” typically means a binding domain comprising a heavy chain variable region, a light chain variable region, a CH1 and a CL region.

In certain embodiments, a multispecific binding moiety of the present disclosure comprises a single Fab domain that binds to PD-1, a single Fab domain that binds to TGF-βRII, and an Fc region. In certain embodiments, a multispecific binding moiety of the present disclosure consists of a single Fab domain that binds to PD-1, a single Fab domain that binds to TGF-βRII, and an Fc region. In certain embodiments, a multispecific binding moiety of the present disclosure consists essentially of a single Fab domain that binds to PD-1, a single Fab domain that binds to TGF-βRII, and an Fc region.

An “Fc region” typically comprises a hinge, CH2, and CH3 region. A suitable hinge includes, but is not limited to, the hinge of which the amino acid sequence is set forth in SEQ ID NO: 68. Suitable CH2 and CH3 regions include, but are not limited to, the CH2 region of which the amino acid sequence is set forth in SEQ ID NO: 70 or 71, and the CH3 region of which the amino acid sequence is set forth in SEQ ID NO: 72, or 73 and 74.

A CL, CH1, CH2, and/or CH3 region may be modified according to methods known in the art in order to obtain favorable antibody characteristics, including for instance to promote heterodimerization of different heavy chains, to improve heavy-light chain pairing, and to enhance or reduce immune cell effector function.

In certain embodiments, the PD-1 binding domain of the multispecific binding moiety blocks PD-1 mediated signaling and the TGF-βRII binding domain of the multispecific binding moiety blocks TGF-βRII-mediated signaling in activated T cells, in particular activated tumor-specific T cells.

In certain embodiments, a multispecific binding moiety of the present disclosure has a higher potency in blocking TGF-βRII-mediated signaling in cells expressing both PD-1 and TGF-βRII than in cells expressing TGF-βRII and no, substantially no, or low levels of, PD-1.

The present disclosure therefore also provides a multispecific binding moiety comprising a PD-1 binding domain and a TGF-βRII binding domain, wherein the multispecific binding moiety has a higher potency in blocking TGF-βRII-mediated signaling in cells expressing both PD-1 and TGF-βRII than in cells expressing TGF-βRII and no, substantially no, or low levels of, PD-1.

In certain embodiments, cells expressing both PD-1 and TGF-βRII are Jurkat-PD-1⁺ cells, such as for instance Jurkat T cells expressing human PD-1 and a luciferase reporter driven by an NFAT response element (NFAT-RE), and the cells expressing TGF-βRII and no PD-1 are Jurkat-PD-1^{null} cells, such as for instance Jurkat-PD-1^{null} cells. Jurkat T cells expressing human PD-1 and a luciferase reporter driven by an NFAT-RE are commercially available, for instance from Promega (cat. no. CS187105—part of kit CS187106 and CS187107); Jurkat-PD-1^{null} cells are publicly available, for instance from the ATCC (cat. no. TIB-152).

In certain embodiments, cells expressing both PD-1 and TGF-βRII are stimulated CD4⁺ or CD8⁺ cells and the cells expressing TGF-βRII and low levels of PD-1 are unstimulated CD4⁺ or CD8⁺ cells. In certain embodiments, the stimulated CD4⁺ or CD8⁺ cells are stimulated with recombinant human TGF-β1.

In certain embodiments, cells expressing both PD-1 and TGF-βRII are HEK-Blue™ TGF-β-PD-1⁺ cells, such as for instance described in Example 7, and the cells expressing TGF-βRII and no PD-1 are HEK-Blue™ TGF-β cells, such as for instance HEK-Blue™ TGF-β cells. HEK-Blue™ TGF-β cells, commercially available from for instance Invivogen (cat. no. hkb-tgfb), are stably transfected human embryonic kidney HEK 293 cells comprising the human TGFBRI, Smad3, and Smad4 genes. They further express a Smad3/4-binding elements (SBE)-inducible SEAP reporter gene.

In certain embodiments no, substantially no, or a low level of PD-1 refers to a level of PD-1 on the cell surface that is undetectable in a suitable assay as set out herein. In certain embodiments, a low level of PD-1 refers to less than 100 PD-1 molecules present on the cell surface. In certain embodiments, the level of PD-1 is measured using quantibrite bead methodology.

For the purposes of the present disclosure, determining if a multispecific binding moiety has a higher potency in blocking TGF-βRII-mediated signaling in cells expressing both PD-1 and TGF-βRII than in cells expressing TGF-βRII and no, substantially no, or low levels of PD-1, is done by using one of the assays described herein.

The potency data of the multispecific binding moieties as provided herein is obtained with the phospho-SMAD2/3 assay as described in Examples 4 and 5 and the isogenic HEK-BLUE-PD-1 TGF-β reporter assay as described in Example 7. Therefore, in certain embodiments, the potency in blocking TGF-βRII-mediated signaling is measured in a phospho-SMAD2/3 assay as described in Example 4 or Example 5.

In brief, the phospho-SMAD2/3 assay as described in Example 4 is performed using Jurkat-PD-1^{null} and Jurkat-PD-1⁺ cells cultured in RPMI/10% FBS. Cells are incubated with test or control antibodies in 6-step serial dilutions (100 μg/ml to 0.001 μg/ml) for one hour at 37° C./5% CO₂. After one hour, human recombinant TGF-β1 is added at a final concentration of 10 ng/ml and the cells are incubated for two more hours at 37° C./5% CO₂. After incubation, cells are washed gently with PBS. Cell lysates are prepared using lysis buffer containing phosphatase inhibitors and protease inhibitors. PhosphoSMAD2/3 levels are determined using ELISA.

In brief, the phospho-SMAD2/3 assay as described in Example 5 is performed using PBMCs from healthy donors, which are stimulated with 1 μg/ml anti-CD3 for 48 hours followed by 16 hour serum deprivation in 0.1% FBS. Stimulated and unstimulated PBMCs are incubated with test and control antibodies for 30 minutes at room temperature. Recombinant human TGF-β1 is added at a final concentration of 1 ng/ml and the cells are incubated for another 30 minutes. Finally, cells are washed twice with PBS and stained for cell surface markers followed by intracellular phosphoSMAD2 staining. Flow cytometry is performed to gate CD4⁺ and CD8+ cells. Phospho-SMAD2 signal is measured in geo mean fluorescence intensity (GMFI) on these cells.

In certain embodiments, the potency in blocking TGF-βRII-mediated signaling is measured in an isogenic HEK-BLUE-PD-1 TGF-β reporter assay as described in Example 7.

In brief, the HEK-BLUE-PD-1 TGF-β reporter assay is performed using HEK-Blue™ TGF-β cells and HEK-Blue™ TGF-β-PD-1⁺ cells at 25000 cells per well. Serial dilutions of test and control antibodies are added and the cells are incubated for one hour at room temperature, followed by the addition of human recombinant TGF-β1 at a final concentration of 1 ng/ml. Cells are incubated at 37° C./5% CO₂ overnight. After incubation, 40 ul of supernatants and 160 ul of re-suspended QUANTI-Blue™ Solution are incubated at 37° C./5% CO₂ for 40 minutes. The quantity of SEAP secreted in the supernatant is assessed using QUANTI-Blue™ Solution. SEAP levels are determined using a spectrophotometer at 650 nm.

In certain embodiments, a multispecific binding moiety of the present disclosure has a potency in blocking TGF-βRII-mediated signaling in cells expressing both PD-1 and TGF-βRII of at least about 10 fold, preferably between about 10-100000 fold, higher than in cells expressing TGF-βRII and no PD-1. In certain embodiments, a multispecific binding moiety of the present disclosure has a potency in blocking TGF-βRII-mediated signaling in cells expressing both PD-1 and TGF-βRII of at least 10 fold, preferably between 10-100000 fold, higher than in cells expressing TGF-βRII and no PD-1. In certain embodiments, the potency in blocking TGF-βRII-mediated signaling is determined in IC50 (μg/ml) in a phospho-SMAD2/3 assay or HEK-BLUE-PD-1 TGF-β reporter assay. In certain embodiments, the potency in blocking TGF-βRII-mediated signaling is determined in IC50 (μg/ml) in a phospho-SMAD2/3 assay. In certain embodiments, the potency in blocking TGF-βRII-mediated signaling is determined in IC50 (μg/ml) in a HEK-BLUE-PD-1 TGF-β reporter assay.

In certain embodiments, the potency of a multispecific binding moiety of the present disclosure in blocking TGF-βRII-mediated signaling in cells expressing TGF-βRII and no, substantially no, or low levels of, PD-1 is lower than the potency of a reference anti-TGF-βRII antibody targeting the same cells, and the potency of the multispecific binding moiety in blocking TGF-βRII-mediated signaling in cells expressing both TGF-βRII and PD-1 is higher than the potency of the reference anti-TGF-βRII antibody targeting the same cells, wherein the reference anti-TGF-βRII antibody is a bivalent monospecific antibody comprising a heavy chain having an amino acid sequence as set forth in SEQ ID NO: 76 and a light chain having an amino acid sequence as set forth in SEQ ID NO: 77.

In certain embodiments, cells expressing both PD-1 and TGF-βRII are Jurkat-PD-1⁺ cells, stimulated CD4⁺ or CD8⁺ cells, or HEK-Blue™ TGF-β-PD-1⁺ cells; the cells expressing TGF-βRII and no, or substantially no, PD-1 are Jurkat-PD-1^{null} cells or are HEK-Blue™ TGF-β cells; and the cells expressing TGF-βRII and low levels of PD-1 are unstimulated CD4⁺ or CD8⁺ cells, as described further herein.

In certain embodiments, the potency of a multispecific binding moiety of the present disclosure in blocking TGF-βRII-mediated signaling in cells expressing both TGF-βRII and PD-1 is at least 10 fold, preferably between 10-100000 fold, higher than the potency of a reference anti-TGF-βRII antibody. In certain embodiments, the potency of a multispecific binding moiety of the present disclosure in blocking TGF-βRII-mediated signaling in cells expressing both TGF-13RII and PD-1 is at least about 10 fold, preferably between about 10-100000 fold, higher than the potency of a reference anti-TGF-βRII antibody. In certain embodiments, the reference TGF-βRII antibody is a bivalent monospecific antibody comprising a heavy chain having an amino acid sequence as set forth in SEQ ID NO: 76 and a light chain having an amino acid sequence as set forth in SEQ ID NO: 77. In certain embodiments, the potency in blocking TGF-βRII-mediated signaling is determined in IC50 (μg/nil) in a phospho-SMAD2/3 assay.

In certain embodiments, a multispecific binding moiety of the present disclosure has a higher activity in reducing tumor volume than a combination of reference antibodies. In certain embodiments, the combination of reference antibodies are two bivalent monospecific antibodies targeting PD-1 and TGF-βRII, wherein the bivalent monospecific antibody targeting PD-1 comprises a heavy chain having an amino acid sequence as set forth in SEQ ID NO: 78 and a light chain having an amino acid sequence as set forth in SEQ ID NO: 79 and the bivalent monospecific antibody targeting anti-TGF-βRII comprises a heavy chain having an amino acid sequence as set forth in SEQ ID NO: 76 and a light chain having an amino acid sequence as set forth in SEQ ID NO: 77.

The present disclosure therefore also provides a multispecific binding moiety comprising a PD-1 binding domain and a TGF-βRII binding domain, wherein the multispecific binding moiety has a higher activity in reducing tumor volume than a combination of reference antibodies. In certain embodiments, the multispecific binding moiety is dosed with a two-fold lower to up to twenty-fold lower number of PD-1 and TGF-βRII binding domains than each of the bivalent monospecific antibodies of the combination of reference antibodies. For example, a multispecific binding moiety, which is monovalent for binding to PD-1 and monovalent for binding to TGF-βRII, when dosed at 1 mg/kg has a higher activity in reducing tumor volume than a combination of reference antibodies, each of which is bivalent for binding to PD-1 or TGF-βRII and each of which is dosed at 10 mg/kg. Also, a multispecific binding moiety, which is monovalent for binding to PD-1 and monovalent for binding to TGF-βRII, when dosed at 10 mg/kg has a higher activity in reducing tumor volume than a combination of reference antibodies, each of which is bivalent for binding to PD-1 or TGF-βRII and each of which is dosed at 10 mg/kg.

In certain embodiments, the combination of reference antibodies are two bivalent monospecific antibodies targeting PD-1 and TGF-βRII, wherein the bivalent monospecific antibody targeting PD-1 comprises a heavy chain having an amino acid sequence as set forth in SEQ ID NO: 78 and a light chain having an amino acid sequence as set forth in SEQ ID NO: 79, and wherein the bivalent monospecific antibody targeting TGF-βRII comprises a heavy chain having an amino acid sequence as set forth in SEQ ID NO: 76 and a light chain having an amino acid sequence as set forth in SEQ ID NO: 77.

In certain embodiments, the activity in reducing tumor volume is determined by measuring tumor volume reduction in an in vivo mouse study, in particular in an in vivo mouse study using MDA-MB-231 xenograft huCD34 NSG mice.

In certain embodiments, a multispecific binding moiety of the present disclosure has a tumor volume reduction that is at least 1.5 fold, preferably between 1.5-100 fold or 2-80 fold or 5-80 fold or 10-80 fold or 15-80 fold or 20-80 fold or 30-80 fold or 40-80 fold or 50-80 fold or 2-60 fold or 5-60 fold or 10-60 fold or 15-60 fold or 20-60 fold or 30-60 fold or 40-60 fold, of the tumor volume reduction of a combination of reference antibodies. In certain embodiments, a multispecific binding moiety of the present disclosure has a tumor volume reduction that is at least about 1.5 fold, preferably about between 1.5-100 fold or 2-80 fold or 5-80 fold or 10-80 fold or 15-80 fold or 20-80 fold or 30-80 fold or 40-80 fold or 50-80 fold or 2-60 fold or 5-60 fold or 10-60 fold or 15-60 fold or 20-60 fold or 30-60 fold or 40-60 fold, of the tumor volume reduction of a combination of reference antibodies. In other words, the multispecific binding moiety of the present disclosure provides a greater reduction in tumor volume than the combination of reference antibodies. In certain embodiments, the combination of reference antibodies are two bivalent monospecific antibodies targeting PD-1 and TGF-βRII, wherein the bivalent monospecific antibody targeting PD-1 comprises a heavy chain having an amino acid sequence as set forth in SEQ ID NO: 78 and a light chain having an amino acid sequence as set forth in SEQ ID NO: 79, and the bivalent monospecific antibody targeting TGF-βRII comprises a heavy chain having an amino acid sequence as set forth in SEQ ID NO: 76 and a light chain having an amino acid sequence as set forth in SEQ ID NO: 77.

In certain embodiments, a multispecific binding moiety of the present disclosure reduces tumor volume when administered as a single agent.

The present disclosure therefore also provides a multispecific binding moiety comprising a PD-1 binding domain and a TGF-βRII binding domain, wherein the multispecific binding moiety induces tumor volume reduction as a single agent.

In certain embodiments, the present disclosure provides several PD-1xTGF-βRII bispecific antibodies as exemplary multispecific binding moieties, the PD-1 binding domains of which comprise a heavy chain variable region having an amino acid sequence selected from SEQ ID NO: 1; 5; 9; 13; 14; 18 and 19, the TGF-βRII binding domains of which comprise a heavy chain variable region having an amino acid sequence selected from SEQ ID NO: 23; 27; 31; 35; 39; 43; 47; 88; and 89, and both the PD-1 and TGF-βRII binding domains comprising the same light chain.

In certain embodiments, the PD-1 binding domain of a multispecific binding moiety of the present disclosure comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;

b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;

c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;

d) heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; or

e) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively.

The heavy chain variable regions of the PD-1 binding domains of a multispecific binding moiety of the present disclosure may comprise a limited number, such as for instance one, two, three, four, five, six, seven, eight, nine, or ten, non-conservative amino acid substitutions, or an unlimited number of conservative amino acid substitutions.

In certain embodiments, the PD-1 binding domain of a multispecific binding moiety of the present disclosure also includes PD-1 binding domain variants thereof, wherein each of the HCDRs may comprise at most three, two, or one amino acid variations. In certain embodiments, only one or two HCDRs may comprise at most three, two, or one non-conservative amino acid variations. In certain embodiments, such variants do not comprise amino acid variations in HCDR3. In certain embodiments, the amino acid variation is a conservative amino acid substitution.

Typically, a conservative amino acid substitution involves a variation of an amino acid with a homologous amino acid residue, which is a residue that shares similar characteristics or properties. Homologous amino acids are known in the art, as are routine methods for making amino acid substitutions in antibody binding domains without significantly impacting binding or function of the antibody, see for instance handbooks like Lehninger (Nelson, David L., and Michael M. Cox. 2017. Lehninger Principles of Biochemistry. 7th ed. New York, N.Y.: W.H. Freeman) or Stryer (Berg, J., Tymoczko, J., Stryer, L. and Stryer, L., 2007. Biochemistry. New York: W.H. Freeman), incorporated herein in its entirety. In determining whether an amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute. For example, the following groups can be determined: (1) non-polar: Ala (A), Gly (G), Val (V), Leu (L), Ile (I), Pro (P), Phe (F), Trp (W), Met (M); (2) uncharged polar: Ser (S), Thr (T), Cys (C), Tyr (Y), Asn (N), Gln (Q); (3) acidic: Asp (D), Glu (E); and (4) basic: Lys (K), Arg (R), His (H). Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (Q), Asn (N), Ser (S), Thr (T), Tyr (Y). Alternatively, amino acid residues may be divided into groups based on common side-chain properties: (1) hydrophobic: Met (M), Ala (A), Val (V), Leu (L), Ile (I); (2) neutral hydrophilic: Cys (C), Ser (S), Thr (T), Asn (N), Gln (Q); (3) acidic: Asp (D), Glu (E); (4) basic: His (H), Lys (K), Arg R); (5) residues that influence chain orientation: Gly (G), Pro (P); and (6) aromatic: Trp (W), Tyr (Y), Phe (F).

The substitution of an amino acid residue with another present in the same group would be preferred. Accordingly, conservative amino acid substitution can involve exchanging a member of one of these classes for another member of that same class. Typically, the variation results in no, or substantially no, loss in binding specificity of the binding domain to its intended target.

Additional types of amino acid variations include variations resulting from somatic hypermutation or affinity maturation. PD-1 binding variants encompassed by the present disclosure include somatically hypermutated or affinity matured heavy chain variable regions, which are heavy chain variable regions derived from the same VH gene segments as the heavy chain variable regions described by sequence herein, the variants having amino acid variations, including non-conservative and/or conservative amino acid substitutions in one, two, or all three HCDRs. Routine methods for affinity maturing antibody binding domains are widely known in the art, see for instance Tabasinezhad M, et al. (Trends in therapeutic antibody affinity maturation: From in-vitro towards next-generation sequencing approaches. Immunol Lett. 2019 August; 212:106-113).

Examples of suitable positions for introducing an amino acid variation include, but are not limited to, the first, second, and/or fourth amino acid of HCDR1; the third, seventh, eighth, ninth, tenth, eleventh, thirteenth, fourteenth, and/or sixteenth amino acid of HCDR2; and/or the sixth and/or thirteenth amino acid of HCDR3.

In certain embodiments, the present disclosure thus also provides a multispecific binding moiety, the PD-1 binding domain of which comprising:
HCDR1 having amino acid sequence X₁X₂FX₃S, wherein
   X₁ can be F, Y, T, or H;
   X₂ can be Y, Q, E, H, or D;
   X₃ can be W, or Y; and/or
HCDR2 having amino acid sequence YIX₁YSGX₂X₃X₄X₅X₆PX₇X₈KX₉, wherein
   X₁ can be Y, V, or I;
   X₂ can be S, or G;
   X₃ can be T, Y, S, H, N, W, L, or Q;
   X₄ can be S, or N;
   X₅ can be F, V, or L;
   X₆ can be N, or S;
   X₇ can be S or A;
   X₈ can be F or L;
   X₉ can be S, T, G, D, R, or N; and/or
HCDR3 having amino acid sequence GGYTGX₁GGDWFDX₂, wherein
   X₁ can be Y, H, V, or A;
   X₂ can be P, V, Y, W, F, T, Q, H, or S.

Other suitable positions for introducing an amino acid variation include, but are not limited to, the second, third, fourth, and/or fifth amino acid of HCDR1; the third, fourth, fifth, sixth, eighth, ninth, tenth, eleventh, twelfth, thirteenth, fourteenth, fifteenth, sixteenth and/or seventeenth amino acid of HCDR2; and/or the first, second, sixth, seventh, ninth, tenth, fourteenth, fifteenth, sixteenth and/or eighteenth amino acid of HCDR3.

In certain embodiments, the present disclosure thus also provides a multispecific binding moiety, the PD-1 binding domain of which comprising:
HCDR1 having amino acid sequence RX₁X₂X₃X₄, wherein
   X₁ can be F, or Y;
   X₂ can be T, A, or V;
   X₃ can be M, L, or V;
   X₄ can be S, H, N, V, or T; and/or
HCDR2 having amino acid sequence WIX₁X₂X₃X₄GX₅X₆X₇X₈X₉X₁₀X₁₁X₁₂X₁₃X₁₄, wherein
   X₁ can be N, or D;
   X₂ can be P, S, or T;
   X₃ can be N, or Q;
   X₄ can be T, or D;
   X₅ can be N, S, T, K, L, or E;
   X₆ can be P, Y, A, H, or F;
   X₇ can be T, or S;
   X₈ can be Y, F, or H;
   X₉ can be A, G, V, or F;
   X₁₀ can be Q, R, N, L, T, or S;
   X₁₁ can be D, A, G, or S;
   X₁₂ can be F, V, or A;
   X₁₃ can be T, K, H, G;
   X₁₄ can be G, N, E, or D; and/or
HCDR3 having amino acid sequence X₁X₂GYCX₃X₄DX₅CYPNX₆X₇X₈DX₉, wherein
   X₁ can be I, S, or V;
   X₂ can be L, Q, or N;
   X₃ can be N, G, S, or D;
   X₄ can be T, S, P, N, or E;
   X₅ can be N, or I;
   X₆ can be W, G, Q, H, W, A, or L;
   X₇ can be I, V, or L;
   X₈ can be F, L, or I;
   X₉ can be Y, S, N, I, R, H, V, T, K, A, or L.

In certain embodiments, a PD-1 binding domain of a multispecific binding moiety of the present disclosure comprises a heavy chain variable region having an amino acid sequence as set forth in any one of SEQ ID NO: 1; 5; 9; 13; 14; 18; 19, or a variant thereof. In certain embodiments, a PD-1 binding domain of a multispecific binding moiety of the present disclosure comprises a heavy chain variable region having an amino acid sequence as set forth in any one of SEQ ID NO: 1; 5; 9; 13; 14; 18; 19, or a variant having at least 80%, at least 85%, at least 90%, or at least 95% sequence identity thereto.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this invention the NEEDLE program from the EMBOSS package is used to determine percent identity of amino acid and nucleic acid sequences (version 2.8.0, EMBOSS: The European Molecular Biology Open Software Suite (2000) Rice, P. LongdenJ. and Bleasby, A. Trends in Genetics 16, (6) pp276-277, http://emboss.bioinformatics.n1/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment.

In certain embodiments, a PD-1 binding domain of a multispecific binding moiety of the present disclosure also comprises PD-1 binding domain variants, which, in addition to the variations in the HCDRs referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a PD-1 binding domain variant of a multispecific binding moiety of the present disclosure comprises no variations in the CDR regions but comprises one or more variations in the framework regions. Such variants have at least 80%, at least 85%, at least 90%, or at least 95% sequence identity to the sequences disclosed herein, and are expected to retain PD-1 binding specificity. Thus, in certain embodiments, a PD-1 binding domain of a multispecific binding moiety of the present disclosure comprises:
a heavy chain variable region having at least 80%, at least 85%, at least 90%, or at least 95% sequence identity to the amino acid sequence as set forth in SEQ ID NO: 1, which heavy chain variable region comprises a HCDR1 amino acid sequence as set forth in SEQ ID NO: 2; a HCDR2 amino acid sequence as set forth in SEQ ID NO: 3; and a HCDR3 amino acid sequence as set forth in SEQ ID NO: 4;
a heavy chain variable region having at least 80%, at least 85%, at least 90%, or at least 95% sequence identity to the amino acid sequence as set forth in SEQ ID NO: 5, which heavy chain variable region comprises a HCDR1 amino acid sequence as set forth in SEQ ID NO: 6; a HCDR2 amino acid sequence as set forth in SEQ ID NO: 7; and a HCDR3 amino acid sequence as set forth in SEQ ID NO: 8;
a heavy chain variable region having at least 80%, at least 85%, at least 90%, or at least 95% sequence identity to the amino acid sequence as set forth in SEQ ID NO: 9, which heavy chain variable region comprises a HCDR1 amino acid sequence as set forth in SEQ ID NO: 10; a HCDR2 amino acid sequence as set forth in SEQ ID NO: 11; and a HCDR3 amino acid sequence as set forth in SEQ ID NO: 12;
a heavy chain variable region having at least 80%, at least 85%, at least 90%, or at least 95% sequence identity to the amino acid sequence as set forth in SEQ ID NO: 13, which heavy chain variable region comprises a HCDR1 amino acid sequence as set forth in SEQ ID NO: 10; a HCDR2 amino acid sequence as set forth in SEQ ID NO: 11; and a HCDR3 amino acid sequence as set forth in SEQ ID NO: 12;
a heavy chain variable region having at least 80%, at least 85%, at least 90%, or at least 95% sequence identity to the amino acid sequence as set forth in SEQ ID NO: 14, which heavy chain variable region comprises a HCDR1 amino acid sequence as set forth in SEQ ID NO: 15; a HCDR2 amino acid sequence as set forth in SEQ ID NO: 16; and a HCDR3 amino acid sequence as set forth in SEQ ID NO: 17;
a heavy chain variable region having at least 80%, at least 85%, at least 90%, or at least 95% sequence identity to the amino acid sequence as set forth in SEQ ID NO: 18, which heavy chain variable region comprises a HCDR1 amino acid sequence as set forth in SEQ ID NO: 15; a HCDR2 amino acid sequence as set forth in SEQ ID NO: 16; and a HCDR3 amino acid sequence as set forth in SEQ ID NO: 17; or
a heavy chain variable region having at least 80%, at least 85%, at least 90%, or at least 95% sequence identity to the amino acid sequence as set forth in SEQ ID NO: 19, which heavy chain variable region comprises a HCDR1 amino acid sequence as set forth in SEQ ID NO: 20; a HCDR2 amino acid sequence as set forth in SEQ ID NO: 21; and a HCDR3 amino acid sequence as set forth in SEQ ID NO: 22.

The binding domains of a multispecific binding moiety of the present disclosure have been generated with a common light chain, in particular with a common light chain referred to as VK1-39/JK1. The binding domains of a multispecific binding moiety of the present disclosure can comprise any suitable light chain, including but not limited to common light chains known in the art. In certain embodiments, the binding domains of a multispecific binding moiety of the present disclosure comprise common light chain VK1-39/JK1, or a variant thereof harboring a limited number, such as for instance one, two, or three, non-conservative amino acid substitutions, or an unlimited number of conservative amino acid substitutions.

In certain embodiments, a PD-1 binding domain of a multispecific binding moiety of the present disclosure comprises a light chain variable region having an amino acid sequence as set forth in SEQ ID NO: 48, or a variant thereof. In certain embodiments, a PD-1 binding domain of a multispecific binding moiety of the present disclosure comprises a light chain variable region having an amino acid sequence as set forth in SEQ ID NO: 48, or a variant having at least 80%, at least 85%, at least 90%, or at least 95% sequence identity thereto.

In certain embodiments, a PD-1 binding domain of a multispecific binding moiety of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51. In certain embodiments, the light chain variable region of a PD-1 binding domain of a multispecific binding moiety of the present disclosure also includes variants thereof, wherein each of the LCDRs may comprise at most three, two, or one amino acid variations. In certain embodiments, the amino acid variation is a conservative amino acid substitution.

In certain embodiments, a PD-1 binding domain of a multispecific binding moiety of the present disclosure also includes PD-1 binding domain variants, which, in addition to the variations in the LCDRs referred to above, comprise one or more variations in the framework regions. A variation is preferably a conservative amino acid substitution. In certain embodiments, a PD-1 binding domain variant of a multispecific binding moiety of the present disclosure comprises no variations in the LCDR regions but comprises one or more variations in the framework regions. Such variants have at least 80%, at least 85%, at least 90%, or at least 95% sequence identity to the sequences disclosed herein. Thus, in certain embodiments, a PD-1 binding domain of a multispecific binding moiety of the present disclosure comprises:
a light chain variable region having at least 80%, at least 85%, at least 90%, or at least 95% sequence identity to the amino acid sequence as set forth in SEQ ID NO: 48, which light chain variable region comprises a LCDR1 amino acid sequence as set forth in SEQ ID NO: 49; a LCDR2 amino acid sequence as set forth in SEQ ID NO: 50; and a LCDR3 amino acid sequence as set forth in SEQ ID NO: 51.

A light chain or light chain variable region comprising these LCDRs and/or light chain variable region can be, for example, the light chain referred to in the art as VK1-39/JK1. This is a common light chain. The term ‘common light chain’ according to the present disclosure refers to a light chain that is capable of pairing with multiple different heavy chains, such as for instance heavy chains having different antigen or epitope binding specificities. A common light chain is particularly useful in the generation of, for instance, bispecific or multispecific antibodies, where antibody production is more efficient when all binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by using well established variations that introduce conservative amino acid changes, changes of amino acids in regions that are known to or are shown to not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 52. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 52, wherein each of the LCDRs may comprise at most three, two, or one amino acid variations, for example substitutions. In certain embodiments, the light chain comprises a light chain variable region having an amino acid sequence as set forth in SEQ ID NO: 52, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 53, SEQ ID NO: 54, and SEQ ID NO: 55; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 56. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 56, wherein each of the LCDRs may comprise at most three, two, or one amino acid variations, for example substitutions. In certain embodiments, the light chain comprises a light chain variable region having an amino acid sequence as set forth in SEQ ID NO: 56, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 57, SEQ ID NO: 58, and SEQ ID NO: 59; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 60. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 60, wherein each of the LCDRs may comprise at most three, two, or one amino acid variations, for example substitutions. In certain embodiments, the light chain comprises a light chain variable region having an amino acid sequence as set forth in SEQ ID NO: 60, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 61, SEQ ID NO: 62, and SEQ ID NO: 63; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 64. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 64, wherein each of the LCDRs may comprise at most three, two, or one amino acid variations, for example substitutions. In certain embodiments, the light chain comprises a light chain variable region having an amino acid sequence as set forth in SEQ ID NO: 64, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 65, SEQ ID NO: 66, and SEQ ID NO: 67.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. An amino acid sequence for VK1-39 is given as SEQ ID NO: 93. This is the sequence of the V-region. The V-region can be combined with one of five J-regions. Suitable VJ-region sequences are indicated as VK1-39/JK1 (SEQ ID NO: 94) and VK1-39/JK5 (SEQ ID NO: 95); alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. An amino acid sequence for VK3-15 is given as SEQ ID NO: 98. This is the sequence of the V-region. The V-region can be combined with one of five J-regions. A suitable VJ-region sequence is indicated as VK3-15/JK1 (SEQ ID NO: 99); alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. An amino acid sequence for VK3-20 is indicated as SEQ ID NO: 100. This is the sequence of the V-region. The V-region can be combined with one of five J-regions. A suitable VJ-region sequence is indicated as VK3-20/JK1 (SEQ ID NO: 101); alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. An amino acid sequence for VL3-21 is given as SEQ ID NO: 102. This is the sequence of the V-region. The V-region can be combined with one of five J-regions. A suitable VJ-region sequence is indicated as VL3-21/JL3 (SEQ ID NO: 103); alternative name is IgVλ3-21/IGJλ3 (nomenclature according to the IMGT database worldwide web at imgt.org). This name is exemplary and encompasses allelic variants of the gene segments.

Further, any light chain variable region of a PD-1 antibody available in the art may be used, as may any other light chain variable region that can readily be obtained, such as from, for instance, an antibody display library by showing antigen binding activity when paired with a PD-1 binding domain of a multispecific binding moiety of the present disclosure.

In certain embodiments, a PD-1 binding domain of a multispecific binding moiety of the present disclosure may further comprise a CH1 and CL region. Any CH1 domain may be used, in particular a human CH1 domain. An example of a suitable CH1 domain is provided by the amino acid sequence provided as SEQ ID NO: 69. Any CL domain may be used, in particular a human CL. An example of a suitable CL domain is provided by the amino acid sequence provided as SEQ ID NO: 75.

In certain embodiments, the TGF-βRII binding domain of a multispecific binding moiety of the present disclosure comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;

b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;

c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;

d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;

e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;

f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or

g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively.

The heavy chain variable regions of the TGF-βRII binding domains of a multispecific binding moiety of the present disclosure may comprise a limited number, such as for instance one, two, or three, non-conservative amino acid substitutions, or an unlimited number of conservative amino acid substitutions.

In certain embodiments, the TGF-βRII binding domain of a multispecific binding moiety of the present disclosure also includes TGF-βRII binding domain variants thereof, wherein each of the HCDRs may comprise at most three, two, or one amino acid variations. In certain embodiments, only one or two HCDRs may comprise at most three, two, or one amino acid variations. In certain embodiments, such variants do not comprise amino acid variations in HCDR3. In certain embodiments, the amino acid variation is a conservative amino acid substitution. A conservative amino acid substitution is as described further herein.

TGF-βRII binding variants encompassed by the present disclosure include somatically hypermutated or affinity matured heavy chain variable regions, which are heavy chain variable regions derived from the same VH gene segment as the heavy chain variable regions described by sequence herein, the variants having amino acid variations, including non-conservative and/or conservative amino acid substitutions in one, two, or all three HCDRs.

In certain embodiments, a TGF-βRII binding domain of a multispecific binding moiety of the present disclosure comprises a heavy chain variable region having an amino acid sequence as set forth in any one of SEQ ID NO: 23; 27; 31; 35; 39; 43; 47; 88; 89, or a variant thereof. In certain embodiments, a TGF-βRII binding domain of a multispecific binding moiety of the present disclosure comprises a heavy chain variable region having an amino acid sequence as set forth in any one of SEQ ID NO: 23; 27; 31; 35; 39; 43; 47; 88; 89, or having at least 80%, at least 85%, at least 90%, or at least 95% sequence identity thereto.

In certain embodiments, a TGF-βRII binding domain of a multispecific binding moiety of the present disclosure also includes TGF-βRII binding domain variants, which, in addition to the variations in the HCDRs referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a TGF-βRII binding domain variant of a multispecific binding moiety of the present disclosure comprises no variations in the CDR regions but comprises one or more variations in the framework regions. Such variants have at least 80%, at least 85%, at least 90%, or at least 95% sequence identity to the sequences disclosed herein, and are expected to retain TGF-βRII binding specificity. Thus, in certain embodiments, a TGF-βRII binding domain of a multispecific binding moiety of the present disclosure comprises:
a heavy chain variable region having at least 80%, at least 85%, at least 90%, or at least 95% sequence identity to the amino acid sequence as set forth in SEQ ID NO: 23, which heavy chain variable region comprises a HCDR1 amino acid sequence as set forth in SEQ ID NO: 24; a HCDR2 amino acid sequence as set forth in SEQ ID NO: 25; and a HCDR3 amino acid sequence as set forth in SEQ ID NO: 26;
a heavy chain variable region having at least 80%, at least 85%, at least 90%, or at least 95% sequence identity to the amino acid sequence as set forth in SEQ ID NO: 27, which heavy chain variable region comprises a HCDR1 amino acid sequence as set forth in SEQ ID NO: 28; a HCDR2 amino acid sequence as set forth in SEQ ID NO: 29; and a HCDR3 amino acid sequence as set forth in SEQ ID NO: 30;
a heavy chain variable region having at least 80%, at least 85%, at least 90%, or at least 95% sequence identity to the amino acid sequence as set forth in SEQ ID NO: 31, which heavy chain variable region comprises a HCDR1 amino acid sequence as set forth in SEQ ID NO: 32; a HCDR2 amino acid sequence as set forth in SEQ ID NO: 33; and a HCDR3 amino acid sequence as set forth in SEQ ID NO: 34;
a heavy chain variable region having at least 80%, at least 85%, at least 90%, or at least 95% sequence identity to the amino acid sequence as set forth in SEQ ID NO: 35, which heavy chain variable region comprises a HCDR1 amino acid sequence as set forth in SEQ ID NO: 36; a HCDR2 amino acid sequence as set forth in SEQ ID NO: 37; and a HCDR3 amino acid sequence as set forth in SEQ ID NO: 38;
a heavy chain variable region having at least 80%, at least 85%, at least 90%, or at least 95% sequence identity to the amino acid sequence as set forth in SEQ ID NO: 39, which heavy chain variable region comprises a HCDR1 amino acid sequence as set forth in SEQ ID NO: 40; a HCDR2 amino acid sequence as set forth in SEQ ID NO: 41; and a HCDR3 amino acid sequence as set forth in SEQ ID NO: 42;
a heavy chain variable region having at least 80%, at least 85%, at least 90%, or at least 95% sequence identity to the amino acid sequence as set forth in SEQ ID NO: 43, which heavy chain variable region comprises a HCDR1 amino acid sequence as set forth in SEQ ID NO: 44; a HCDR2 amino acid sequence as set forth in SEQ ID NO: 45; and a HCDR3 amino acid sequence as set forth in SEQ ID NO: 46;
a heavy chain variable region having at least 80%, at least 85%, at least 90%, or at least 95% sequence identity to the amino acid sequence as set forth in SEQ ID NO: 47, which heavy chain variable region comprises a HCDR1 amino acid sequence as set forth in SEQ ID NO: 90; a HCDR2 amino acid sequence as set forth in SEQ ID NO: 91; and a HCDR3 amino acid sequence as set forth in SEQ ID NO: 92;
a heavy chain variable region having at least 80%, at least 85%, at least 90%, or at least 95% sequence identity to the amino acid sequence as set forth in SEQ ID NO: 88, which heavy chain variable region comprises a HCDR1 amino acid sequence as set forth in SEQ ID NO: 44; a HCDR2 amino acid sequence as set forth in SEQ ID NO: 45; and a HCDR3 amino acid sequence as set forth in SEQ ID NO: 46; or
a heavy chain variable region having at least 80%, at least 85%, at least 90%, or at least 95% sequence identity to the amino acid sequence as set forth in SEQ ID NO: 89, which heavy chain variable region comprises a HCDR1 amino acid sequence as set forth in SEQ ID NO: 90; a HCDR2 amino acid sequence as set forth in SEQ ID NO: 91; and a HCDR3 amino acid sequence as set forth in SEQ ID NO: 92.

Any light chain variable region of a TGF-βRII antibody available in the art may be used, for example as described herein, as may any other light chain variable region that can readily be obtained, such as from, for instance, an antibody display library by showing antigen binding activity when paired with a TGF-βRII binding domain of a multispecific binding moiety of the present disclosure. In certain embodiments, the TGF-βRII binding domain of a multispecific binding moiety of the present disclosure comprises the same or substantially the same light chain as the PD-1 binding domain.

In certain embodiments, the TGF-βRII binding domain of a multispecific binding moiety of the present disclosure comprises a light chain variable region having an amino acid sequence as set forth in SEQ ID NO: 48, or a variant thereof. In certain embodiments, the TGF-βRII binding domain of a multispecific binding moiety of the present disclosure comprises a light chain variable region having an amino acid sequence as set forth in SEQ ID NO: 48, or a variant having at least 80%, at least 85%, at least 90%, or at least 95% sequence identity thereto.

In certain embodiments, the TGF-βRII binding domain of a multispecific binding moiety of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively. In certain embodiments, the light chain variable region of a TGF-βRII binding domain of a multispecific binding moiety of the present disclosure also includes variants thereof, wherein each of the LCDRs may comprise at most three, two, or one conservative or non-conservative amino acid variations. In certain embodiments, the amino acid variation is a conservative amino acid substitution.

In certain embodiments, a TGF-βRII binding domain of a multispecific binding moiety of the present disclosure may further comprise a CH1 and CL region. Any CH1 domain may be used, in particular a human CH1 domain. An example of a suitable CH1 domain is provided by the amino acid sequence provided as SEQ ID NO: 69. Any CL domain may be used, in particular a human CL. An example of a suitable CL domain is provided by the amino acid sequence provided as SEQ ID NO: 75.

The present invention thus also provides a multispecific binding moiety comprising a PD-1 binding domain and a TGF-βRII binding domain, wherein the PD-1 binding domain comprises a heavy chain variable region, and optionally a light chain variable region and CH1 and CL regions, as described herein. In certain embodiments, the multispecific binding moiety further comprises a TGF-βRII binding domain that comprises a heavy chain variable region, and optionally a light chain variable region and CH1 and CL regions, as described herein.

The present invention thus also provides a multispecific binding moiety comprising a PD-1 binding domain and a TGF-βRII binding domain, wherein the TGF-βRII binding domain comprises a heavy chain variable region, and optionally a light chain variable region and CH1 and CL regions, as described herein. In certain embodiments, the multispecific binding moiety further comprises a PD-1 binding domain that comprises a heavy chain variable region, and optionally a light chain variable region and CH1 and CL regions, as described herein.

In certain embodiments, any PD-1 binding domain disclosed herein can be combined with any TGF-βRII binding domain disclosed herein to produce a multispecific binding moiety of the present disclosure. The present disclosure thus provides exemplary multispecific binding moieties PB1-PB18, as presented in Table 1.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein each of the HCDRs may comprise at most three, two, or one amino acid variations, for example, substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively,

wherein each of the HCDRs may comprise at most three, two, or one amino acid variations, for example substitutions. In certain embodiments, the HCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively,

wherein the PD-1 binding domain and TGF-βRII binding domain comprise a light chain CDR1 (LCDR1) having an amino acid sequence as set forth in SEQ ID NO: 49, light chain CDR2 (LCDR2) having an amino acid sequence as set forth in SEQ ID NO: 50, and light chain CDR3 (LCDR3) having an amino acid sequence as set forth in SEQ ID NO: 51, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 18, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 14, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 31, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 39, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 14, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 27, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 13, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 47, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 43, or a heavy chain variable region that has at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a multispecific binding moiety
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 18, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 14, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 31, or a heavy chain variable region that has at least 80%, at least 85%, at least 90%, at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 39, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety
a PD-1 binding domain as described herein comprising a heavy chain variable region having an amino acid sequence as set forth in SEQ ID NO: 14, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety comprising:
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety
a PD-1 binding domain as described herein comprising a heavy variable region having an amino acid sequence as set forth in SEQ ID NO: 9, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 27, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety comprising:
a PD-1 binding domain as described herein comprising a heavy variable region having an amino acid sequence as set forth in SEQ ID NO: 13, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 47, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a multispecific binding moiety
a PD-1 binding domain as described herein comprising a heavy chain variable region having an amino acid sequence as set forth in SEQ ID NO: 19, or a heavy chain variable region that has at least 80%, at least 85%, at least 90%, or at least 95% sequence identity thereto; and
a TGF-βRII binding domain as described herein comprising a heavy chain variable region having an amino acid sequence as set forth in SEQ ID NO: 43, or a heavy chain variable region that has at least 80%, at least 85%, at least 90%, or at least 95% sequence identity thereto,

wherein the PD-1 binding domain and TGF-βRII binding domain comprise a light chain variable region having an amino acid sequence as set forth in SEQ ID NO: 48, or a light chain variable region that has at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

Further provided herein are nucleic acids useful for producing a multispecific binding moiety of the present disclosure. In certain embodiments, such nucleic acids comprise a nucleic acid sequence encoding the heavy chain variable region of a PD-1 binding domain as described herein and a nucleic acid sequence encoding the heavy chain variable region of a TGF-βRII binding domain as described herein. In certain embodiments, a nucleic acid of the present disclosure may further comprise a nucleic acid sequence encoding a CH1 region and preferably a hinge, CH2 and CH3 region. In certain embodiments, a nucleic acid of the present disclosure may further comprise at least one nucleic acid sequence encoding a light chain variable region, and preferably a CL region. In certain embodiments, the light chain variable region can be a common light chain variable region as described herein.

Further provided herein are vectors comprising nucleic acids of the present disclosure useful for producing a multispecific binding moiety of the present disclosure. In certain embodiments, such vectors comprise a nucleic acid sequence encoding the heavy chain variable region of a PD-1 binding domain as described herein and a nucleic acid sequence encoding the heavy chain variable region of a TGF-βRII binding domain as described herein. In certain embodiments, a vector of the present disclosure may further comprise a nucleic acid sequence encoding a CH1 region and preferably a hinge, CH2 and CH3 region. In certain embodiments, a vector of the present disclosure may further comprise at least one nucleic acid sequence encoding a light chain variable region, and preferably a CL region. In certain embodiments, the light chain variable region can be a common light chain variable region as described herein.

The present disclosure also provides a cell comprising a nucleic acid sequence, for example a vector, encoding the heavy chain variable region of a PD-1 binding domain as described herein and a nucleic acid sequence encoding the heavy chain variable region of a TGF-βRII binding domain as described herein. In certain embodiments, a cell of the present disclosure may further comprise a nucleic acid sequence, for example a vector, encoding a CH1 region and preferably a hinge, CH2 and CH3 region. In certain embodiments, a cell of the present disclosure may further comprise at least one nucleic acid sequence, for example a vector, encoding a light chain variable region, and preferably a CL region. In certain embodiments, the light chain variable region can be a common light chain variable region as described herein.

The present disclosure also provides a cell producing a multispecific binding moiety as described herein. In certain embodiments, such cell can be a recombinant cell, which has been transformed with nucleic acid, for example a vector, of the present disclosure. In certain embodiments, a cell of the present disclosure comprises a nucleic acid sequence, for example a vector, encoding the heavy chain variable region of a PD-1 binding domain as described herein and a nucleic acid sequence encoding the heavy chain variable region of a TGF-βRII binding domain as described herein. In certain embodiments, a cell of the present disclosure further comprises a nucleic acid sequence, for example a vector, encoding a CH1 region and preferably a hinge, CH2 and CH3 region. In certain embodiments, a cell of the present disclosure further comprises at least one nucleic acid sequence, for example a vector, encoding a light chain variable region, in particular a light chain variable region as described herein, and preferably a CL region.

The present disclosure further provides a cell producing a multispecific binding moiety as described herein.

In certain embodiments, the present disclosure provides a pharmaceutical composition comprising an effective amount of a multispecific binding moiety as described herein, and optionally a pharmaceutically acceptable carrier.

In certain embodiments, the present disclosure provides a multispecific binding moiety as described herein, and a pharmaceutical composition as described herein, for use in therapy.

In certain embodiments, the present disclosure provides a multispecific binding moiety as described herein, or the pharmaceutical composition as described herein, for use in the treatment of cancer.

In certain embodiments, the present disclosure provides a method for treating a disease, comprising administering an effective amount of a multispecific binding moiety as described herein, or the pharmaceutical composition as described herein, to an individual in need thereof.

In certain embodiments, the present disclosure provides a method for treating cancer, comprising administering an effective amount of a multispecific binding moiety as described herein, or the pharmaceutical composition as described herein, to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like, and in particular to a human subject having cancer.

The terms “treat,” “treating,” and “treatment,” as used herein, refer to any type of intervention or process performed on or administering an active agent or combination of active agents to a subject with the objective of curing or improving a disease or symptom thereof or which produces a positive therapeutic response. As used herein, “positive therapeutic response” refers to a treatment producing a beneficial effect, e.g. reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease, such as, for example, amelioration of at least one symptom of a disease or disorder, e.g. cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease in the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that treats a disease, such as cancer. In some embodiments, a therapeutic amount is an amount sufficient to delay tumor development. In some embodiments, a therapeutic amount is an amount sufficient to prevent or delay tumor recurrence.

As used herein, an effective amount of the agent or composition is one that, for example, may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual, which can be readily evaluated by the ordinarily skilled physician or other health care worker.

An effective amount can be administered to a subject in one or more administrations.

An effective amount can also include an amount that balances any toxic or detrimental effects of the agent or combination of agents and the beneficial effects.

The term “agent” refers to a therapeutically active substance, in the present case a multispecific binding moiety of the present disclosure, or a pharmaceutical composition of the present disclosure.

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. An antigen binding site of a multispecific binding moieties of the disclosure can bind the antigen and a variety of variants thereof, such as those expressed by some antigen positive immune or tumor cells. HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensembl provides the accession number with which information on the gene or protein encoded by the gene can be obtained from the Ensembl database. Ensembl is a joint project between EMBL-EBI and the Wellcome Trust Sanger Institute to develop a software system which produces and maintains automatic annotation on selected eukaryotic genomes.

When herein reference is made to a gene or a protein, the reference is preferably to the human form of the gene or protein. When herein reference is made to a gene or protein reference is made both to the natural gene or protein and to variant forms of the gene or protein as can be detected in tumors, cancers and the like, preferably as can be detected in human tumors, cancers and the like.

### Clauses

1. A multispecific binding moiety comprising a PD-1 binding domain and a TGF-βRII binding domain, wherein the PD-1 binding domain blocks PD-1 mediated signaling and the TGF-βRII binding domain blocks TGF-βRII-mediated signaling.

2. The multispecific binding moiety according to clause 1, wherein the PD-1 binding domain blocks PD-1 mediated signaling and the TGF-βRII binding domain blocks TGF-βRII-mediated signaling in activated T cells.

3. The multispecific binding moiety according to clause 1 or 2, wherein the PD-1 binding domain blocks PD-1 mediated signaling and the TGF-βRII binding domain blocks TGF-βRII-mediated signaling in activated tumor-specific T cells.

4. A multispecific binding moiety comprising a Fab domain that specifically binds to PD-1 and a Fab domain that specifically binds to TGF-βRII.

5. The multispecific binding moiety according to any one of the preceding clauses, wherein the multispecific binding moiety consists of a single Fab domain that specifically binds to PD-1, a single Fab domain that specifically binds to TGF-βRII, and an Fc region.

6. The multispecific binding moiety according to any one of the preceding clauses, wherein the multispecific binding moiety has a higher potency in blocking TGF-βRII-mediated signaling in cells expressing both PD-1 and TGF-βRII than in cells expressing TGF-βRII and no, substantially no, or low levels of PD-1.

7. A multispecific binding moiety comprising a PD-1 binding domain and a TGF-βRII binding domain, wherein the multispecific binding moiety has a higher potency in blocking TGF-βRII-mediated signaling in cells expressing both PD-1 and TGF-βRII than in cells expressing TGF-βRII and no, substantially no, or low levels of PD-1.

8. A multispecific binding moiety comprising a PD-1 binding domain and a TGF-βRII binding domain, wherein the multispecific binding moiety has a higher activity in reducing tumor volume than a combination of reference antibodies, wherein the combination of reference antibodies are two bivalent monospecific antibodies targeting PD-1 and TGF-βRII, wherein the bivalent monospecific antibody targeting PD-1 comprises a heavy chain having an amino acid sequence as set forth in SEQ ID NO: 78 and a light chain having an amino acid sequence as set forth in SEQ ID NO: 79, and the bivalent monospecific antibody targeting TGF-βRII comprises a heavy chain having an amino acid sequence as set forth in SEQ ID NO: 76 and a light chain having an amino acid sequence as set forth in SEQ ID NO: 77.

9. The multispecific binding moiety according to clause 8, wherein the activity in reducing tumor volume is determined by measuring tumor volume reduction in an in vivo mouse study, in particular in an in vivo mouse study using MDA-MB-231 xenograft huCD34 NSG mice.

10. The multispecific antibody or variant thereof according to clause 8 or 9, wherein a higher activity in reducing tumor volume is a tumor volume reduction of at least about 1.5 fold, preferably between about 1.5-100 fold, of the tumor volume reduction of the combination of reference antibodies.

11. The multispecific binding moiety according to any one of the preceding clauses, wherein the cells expressing both PD-1 and TGF-βRII are activated T cells.

12. The multispecific binding moiety according to any one of the preceding clauses, wherein the cells expressing both PD-1 and TGF-βRII are activated tumor-specific T cells.

13. The multispecific binding moiety according to any one of the preceding clauses, wherein the cells expressing both PD-1 and TGF-βRII are Jurkat-PD-1⁺ cells and the cells expressing TGF-βRII and no, or substantially no, PD-1 are Jurkat-PD-1″¹¹ cells.

14. The multispecific binding moiety according to any one of the preceding clauses, wherein the cells expressing both PD-1 and TGF-βRII are activated CD4⁺ and/or CD8⁺ cells and the cells expressing TGF-βRII and no, substantially no, or low levels of PD-1 are non-activated CD4⁺ and/or CD8⁺ cells.

15. The multispecific binding moiety according to any one of the preceding clauses, wherein the cells expressing both PD-1 and TGF-βRII are HEK-Blue TGF-β-PD-1⁺ cells and the cells expressing TGF-βRII and no, substantially no, or low levels of PD-1 are HEK-Blue TGF-β cells.

16. The multispecific binding moiety according to any one of the preceding clauses, wherein the potency in blocking TGF-βRII-mediated signaling is measured in a phospho-SMAD2/3 assay.

17. The multispecific binding moiety according to any one of the preceding clauses, wherein the potency in blocking TGF-βRII-mediated signaling is measured in an isogenic PD-1-TGF-β reporter assay.

18. The multispecific binding moiety according to any one of the preceding clauses, wherein the potency in blocking TGF-βRII-mediated signaling in cells expressing both PD-1 and TGF-βRII is at least about 200 fold or 500 fold or 1000 fold or 5000 fold or 10000 fold or 15000 fold or 20000 fold or 30000 fold, or between about 200-30000 fold or 500-30000 fold or 1000-30000 fold or 5000-30000 fold or 10000-30000 fold or 200-20000 fold or 200-15000 fold, higher than in cells expressing TGF-βRII and no, or substantially, or low levels of PD-1.

19. The multispecific binding moiety according to any one of the preceding clauses, wherein the potency of the multispecific binding moiety in blocking TGF-βRII-mediated signaling in cells expressing TGF-βRII and no PD-1 is lower than the potency of a reference anti-TGF-βRII antibody and the potency of the multispecific binding moiety in blocking TGF-βRII-mediated signaling in cells expressing both TGF-βRII and PD-1 is higher than the potency of the reference anti-TGF-βRII antibody, wherein the reference anti-TGF-βRII antibody is a bivalent monospecific antibody comprising a heavy chain having an amino acid sequence as set forth in SEQ ID NO: 76 and a light chain having an amino acid sequence as set forth in SEQ ID NO: 77.

20. The multispecific binding moiety according to any one of the preceding clauses, wherein the potency of the multispecific binding moiety in blocking TGF-βRII-mediated signaling in cells expressing both TGF-βRII and PD-1 is at least about 100 fold or 200 fold, preferably between about 100-20000 fold or 100-15000 fold or 100-12000 fold or 200-20000 fold or 200-15000 fold or 200-12000 fold, higher than the potency of the reference anti-TGF-βRII antibody.

21. The multispecific binding moiety according to any one of the preceding clauses, wherein the PD-1 binding domain and TGF-βRII binding domain are Fab domains.

22. The multispecific binding moiety according to any one of the preceding clauses, wherein the multispecific binding moiety is a bispecific antibody.

23. The multispecific binding moiety according to any one of the preceding clauses, wherein the multispecific binding moiety is an IgG1 bispecific antibody.

24. The multispecific binding moiety according to any one of the preceding clauses, wherein the PD-1 binding domain and TGF-βRII binding domain each comprise a light chain comprising a light chain variable region of a light chain that is capable of pairing with multiple heavy chains having different epitope specificities.

25. The multispecific binding moiety according to any one of the preceding clauses, wherein the PD-1 binding domain and TGF-βRII binding domain comprise the same light chain.

26. The multispecific binding moiety according to any one of the preceding clauses, wherein the PD-1 binding domain comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;

b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;

c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;

d) heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; or

e) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively;

wherein each of the HCDRs may comprise at most three, two, or one amino acid variations.

27. The multispecific binding moiety according to any one of the preceding clauses, wherein the TGF-βRII binding domain comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;

b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;

c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;

d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;

e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;

f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or

g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,

wherein each of the HCDRs may comprise at most three, two, or one amino acid variations.

28. A multispecific binding moiety comprising a PD-1 binding domain and a TGF-βRII binding domain, wherein the PD-1 binding domain comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;

b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;

c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;

d) heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; or

e) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively;

wherein each of the HCDRs may comprise at most three, two, or one amino acid variations.

29. A multispecific binding moiety comprising a PD-1 binding domain and a TGF-βRII binding domain, wherein the TGF-βRII binding domain comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;

b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;

c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;

d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;

e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;

f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or

g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,

wherein each of the HCDRs may comprise at most three, two, or one amino acid variations.

30. The multispecific binding moiety according to any one of the preceding clauses, wherein the PD-1 binding domain comprises a heavy chain variable region having an amino acid sequence as set forth in any one of SEQ ID NO: 1; 5; 9; 13; 14; 18; 19, or having at least 80%, preferably 85%, more preferably 90%, or most preferably 95% sequence identity thereto.

31. The multispecific binding moiety according to any one of the preceding clauses, wherein the PD-1 binding domain comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, wherein each of the LCDRs may comprise at most three, two, or one amino acid variations.

32. The multispecific binding moiety according to any one of the preceding clauses, wherein the PD-1 binding domain comprises a light chain variable region having an amino acid sequence as set forth in SEQ ID NO: 48, or having at least 80%, preferably 85%, more preferably 90%, or most preferably 95% sequence identity thereto.

33. The multispecific binding moiety according to any one of the preceding clauses, wherein the TGF-βRII binding domain comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;

b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;

c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;

d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;

e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;

f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or

g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,

wherein each of the HCDRs may comprise at most three, two, or one amino acid variations.

34. The multispecific binding moiety according to any one of the preceding clauses, wherein the TGF-βRII binding domain comprises a heavy chain variable region having an amino acid sequence as set forth in any one of SEQ ID NO: 23; 27; 31; 35; 39; 43; 47; 88; 89, or having at least 80%, preferably 85%, more preferably 90%, or most preferably 95% sequence identity thereto.

35. The multispecific binding moiety according to any one of the preceding clauses, wherein the TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, wherein each of the LCDRs may comprise at most three, two, or one amino acid variations.

36. The multispecific binding moiety according to any one of the preceding clauses, wherein the TGF-βRII binding domain comprises a light chain variable region having an amino acid sequence as set forth in SEQ ID NO: 48, or having at least 80%, preferably 85%, more preferably 90%, or most preferably 95% sequence identity thereto.

37. A multispecific binding moiety comprising a PD-1 binding domain and a TGF-βRII binding domain that competes with a multispecific binding moiety of any one of the preceding clauses for binding to PD-1 and/or TGF-βRII.

38. A pharmaceutical composition comprising an effective amount of the multispecific binding moiety according to any one of the preceding clauses, and a pharmaceutically acceptable carrier.

39. The multispecific binding moiety according to any one of clauses 1-37, or the pharmaceutical composition according to clause 38, for use in therapy.

40. The multispecific binding moiety according to any one of clauses 1-37, or the pharmaceutical composition according to clause 38, for use in the treatment of a disease associated with a suppressed immune system.

41. The multispecific binding moiety according to any one of clauses 1-37, or the pharmaceutical composition according to clause 38, for use in the treatment of cancer.

42. Use of a multispecific binding moiety according to any one of clauses 1-37, or the pharmaceutical composition according to clause 38, for the manufacture of a medicament for use in the treatment of a disease associated with a suppressed immune system.

43. Use of a multispecific binding moiety according to any one of clauses 1-37, or the pharmaceutical composition according to clause 38, for the manufacture of a medicament for use in the treatment of cancer.

44. A method for treating a disease, comprising administering an effective amount of a multispecific binding moiety according to any one of clauses 1-37, or the pharmaceutical composition according to clause 38, to a human subject in need thereof.

45. A method for treating a disease associated with a suppressed immune system, comprising administering an effective amount of a multispecific binding moiety according to any one of clauses 1-37, or the pharmaceutical composition according to clause 38, to a human subject in need thereof.

46. A method for treating cancer, comprising administering an effective amount of a multispecific binding moiety according to any one of clauses 1-37, or the pharmaceutical composition according to clause 38, to a human subject in need thereof.

47. A cell comprising a nucleic acid sequence encoding the heavy chain variable region of a PD-1 binding domain as defined in clause 28 or 30 and a nucleic acid sequence encoding the heavy chain variable region of a TGF-βRII binding domain as defined in clause 29 or 34.

48. The cell according to clause 47, wherein the cell further comprises a nucleic acid sequence encoding a CH1 region and preferably a hinge, CH2 and CH3 region.

49. The cell according to clause 47 or 48, wherein the cell further comprises at least one nucleic acid sequence encoding a light chain variable region, in particular a light chain variable region as defined in clause 34 or 35, and preferably a CL region.

50. A cell producing a multispecific binding moiety according to any one of clauses 1-37.

### BRIEF DESCRIPTION OF THE DRAWINGS

The following naming conventions are used herein as follows. In the Figures, bivalent monospecific antibodies are indicated in the format SEQ ID NO: A/SEQ ID NO: B, where SEQ ID NO: A refers to the heavy chain of both binding domains and SEQ ID NO: B refers to the light chain of both binding domains.

Bivalent bispecific antibodies are indicated in the format SEQ ID NO: A×SEQ ID NO: B, where both SEQ ID NO: A and B refer to heavy chain variable sequences. Each binding domain of the bispecific antibodies comprises the same light chain.

Bivalent monospecific reference antibodies pembrolizumab, nivolumab, and TGF1 analog are indicated in the format SEQ ID NO: A/SEQ ID NO: B, where SEQ ID NO: A refers to the respective heavy chain sequence and SEQ ID NO: B refers to the respective light chain sequence. A combination of pembrolizumab and TGF1 analog is indicated in the format SEQ ID NO: A/SEQ ID NO: B+SEQ ID NO: C/SEQ ID NO: D, where SEQ ID NO: A refers to the heavy chain sequence and SEQ ID NO: B refers to the light chain sequence of either pembrolizumab or TGF1 analog, and SEQ ID NO: C to the heavy chain sequence and SEQ ID NO: D to the light chain sequence of the other.

Reference PD-L1-TGF-β TRAP molecule analog, which is an analog of Bintrafusp alfa, is indicated in the format SEQ ID NO: A/SEQ ID NO: B, where SEQ ID NO: A refers to the heavy chain sequence including a (G₄S)₄G linker and extracellular domain of TGF-βRII, and SEQ ID NO: B refers to the light chain sequence. The reference PD-L1-TGF-β TRAP molecule analog comprises two PD-L1 binding domains and two TGF-βRII extracellular domains.

FIGS. 1A and 1B—Percentage inhibition of PD-1-mediated SHP recruitment by bispecific antibodies and control antibodies as measured in a PD-1-SHP Recruitment Assay. FIG. 1A) Bispecific antibodies are: SEQ ID NO: 39×SEQ ID NO: 9 and SEQ ID NO: 35×SEQ ID NO: 5. Control antibodies are: pembrolizumab analog—SEQ ID NO: 78/SEQ ID NO: 79; PD-L1-TGF-β TRAP molecule analog—SEQ ID NO: 80/SEQ ID NO: 81, and TGF1 analog—SEQ ID NO: 76/SEQ ID NO: 77. FIG. 1B) Bispecific antibodies are: SEQ ID NO: 23×SEQ ID NO: 18; SEQ ID NO: 47×SEQ ID NO: 13; SEQ ID NO: 88×SEQ ID NO: 13; SEQ ID NO: 89×SEQ ID NO: 13; SEQ ID NO: 23×SEQ ID NO: 14; and SEQ ID NO: 43×SEQ ID NO: 9. Control antibodies are: pembrolizumab analog—SEQ ID NO: 78/SEQ ID NO: 79; and RSV IgG1—SEQ ID NO: 86/SEQ ID NO: 87.

FIGS. 2A-2C—Fold induction of T cell activation by bispecific antibodies and control antibodies as measured in a PD-1-NFAT Reporter Assay. FIG. 2A) Bispecific antibodies are: SEQ ID NO: 39×SEQ ID NO: 9 and SEQ ID NO: 35×SEQ ID NO: 5. Control antibodies are: pembrolizumab analog—SEQ ID NO: 78/SEQ ID NO: 79; PD-L1-TGF-β TRAP molecule analog—SEQ ID NO: 80/SEQ ID NO: 81, and TGF1 analog—SEQ ID NO: 76/SEQ ID NO: 77. FIG. 2B) Bispecific antibodies are: SEQ ID NO: 47×SEQ ID NO: 13; SEQ ID NO: 88×SEQ ID NO: 13; and SEQ ID NO: 89×SEQ ID NO: 13. Control antibodies are: pembrolizumab analog—SEQ ID NO: 78/SEQ ID NO: 79; and RSV IgG1—SEQ ID NO: 86/SEQ ID NO: 87. FIG. 2C) Bispecific antibodies are: SEQ ID NO: 23×SEQ ID NO: 18; SEQ ID NO: 23×SEQ ID NO: 14; and SEQ ID NO: 43×SEQ ID NO: 9. Control antibodies are: pembrolizumab analog—SEQ ID NO: 78/SEQ ID NO: 79; and RSV IgG1—SEQ ID NO: 86/SEQ ID NO: 87.

FIGS. 3A-3N—Inhibition of phosphoSMAD2/3 by bispecific antibodies and control antibodies in Jurkat-PD-1^{null} (FIGS. 3A-3G) cells and Jurkat-PD-1⁺ cells (FIGS. 3H-3N). These graphs show phosphoSMAD2/3 levels in lysates of Jurkat-PD-1^{null} cells and Jurkat-PD-1⁺ cells incubated with bispecific antibodies or control antibodies. “No TGF-β1” indicates the background phosphoSMAD2/3 level when no TGF-β ligand is added as measured in the absence of bispecific antibodies; “10 ng/ml TGF-β1” indicates the maximum phosphoSMAD2/3 level when 10 ng/ml TGF-β ligand is added in the absence of bispecific antibodies. Control antibodies are: RSV IgG1—SEQ ID NO: 86/SEQ ID NO: 87; TGF1 analog—SEQ ID NO: 76/SEQ ID NO: 77; and TGF-βRIIxRSV antibodies comprising a TGF-βRII binding domain comprising a heavy chain variable region amino acid sequence as set forth in SEQ ID NO: 23, 31, 39, 27, 35, or 43; a RSV binding domain comprising a heavy chain variable region amino acid sequence as set forth in SEQ ID NO: 86; and a common light chain comprising a light chain variable region amino acid sequence as set forth in SEQ ID NO: 48 and a light chain constant region amino acid sequence as set forth in SEQ ID NO: 75. Each data point represents the mean absorbance of corresponding duplicates. FIGS. 3A and 3H: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 23; FIGS. 3B and 3I: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 31; FIGS. 3C and 3J: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 39; FIGS. 3D and 3K: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 27, FIGS. 3E and 3L: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 35; FIGS. 3F and 3M: bispecific antibodies comprising a PD-1 binding domain with a heavy chain variable region having an amino acid sequence as set forth in SEQ ID NO: 1, 5, 9, 14, or 19, and a TGF-βRII binding domain with a heavy chain variable region having an amino acid sequence as set forth in SEQ ID NO: 43; FIGS. 3G and 3N: Bispecific antibodies are: SEQ ID NO: 23×SEQ ID NO: 9; SEQ ID NO: 23×SEQ ID NO: 14; SEQ ID NO: 43×SEQ ID NO: 9; SEQ ID NO: 23×SEQ ID NO: 13; SEQ ID NO: 23×SEQ ID NO: 18; SEQ ID NO: 47×SEQ ID NO: 13; SEQ ID NO: 88×SEQ ID NO: 13; and SEQ ID NO: 89×SEQ ID NO: 13. Control antibodies are: RSV IgG1—SEQ ID NO: 86/SEQ ID NO: 87; TGF1 analog—SEQ ID NO: 76/SEQ ID NO: 77; and pembrolizumab—SEQ ID NO: 78/SEQ ID NO: 79.

FIGS. 4A-4H—Intracellular phosphoSMAD2 measurement by Flow Cytometry. These graphs show intracellular phosphoSMAD2 levels of stimulated and unstimulated CD4⁺ and CD8⁺ T cells incubated with bispecific antibodies or control antibodies. Bispecific antibodies are: SEQ ID NO: 23×SEQ ID NO: 18 and SEQ ID NO: 47×SEQ ID NO: 13. Control antibodies are: RSV IgG1—SEQ ID NO: 86/SEQ ID NO: 87; TGF1 analog—SEQ ID NO: 76/SEQ ID NO: 77; and pembrolizumab—SEQ ID NO: 78/SEQ ID NO: 79. FIG. 4A: donor A stimulated CD4⁺ T cells; FIG. 4B: donor A stimulated CD8⁺ T cells; FIG. 4C: donor A unstimulated CD4⁺ T cells; FIG. 4D: donor A unstimulated CD8⁺ T cells; FIG. 4E: donor B stimulated CD4⁺ T cells; FIG. 4F: donor B stimulated CD8⁺ T cells; FIG. 4G: donor B unstimulated CD4⁺ T cells; FIG. 4H: donor B unstimulated CD8⁺ T cells.

FIGS. 5A-5E—Measurement of cytokine production induced by bispecific antibodies or control antibodies in an exhausted MLR assay. Bispecific antibodies tested are: SEQ ID NO: 23×SEQ ID NO: 9; SEQ ID NO: 23×SEQ ID NO: 14; SEQ ID NO: 23×SEQ ID NO: 19; SEQ ID NO: 31×SEQ ID NO: 14; SEQ ID NO: 39×SEQ ID NO: 9; SEQ ID NO: 35×SEQ ID NO: 9; SEQ ID NO: 35×SEQ ID NO: 14; SEQ ID NO: 35×SEQ ID NO: 19; SEQ ID NO: 27×SEQ ID NO: 9; SEQ ID NO: 43×SEQ ID NO: 9; SEQ ID NO: 43×SEQ ID NO: 19; SEQ ID NO: 23×SEQ ID NO: 13; SEQ ID NO: 23×SEQ ID NO: 18; SEQ ID NO: 47×SEQ ID NO: 13; SEQ ID NO: 88×SEQ ID NO: 13; and SEQ ID NO: 89×SEQ ID NO: 13. Control antibodies are: RSV IgG1—SEQ ID NO: 86/SEQ ID NO: 87; TGF1 analog—SEQ ID NO: 76/SEQ ID NO: 77; a combination of pembrolizumab and TGF1 analog—SEQ ID NO: 78/SEQ ID NO: 79+SEQ ID NO: 76/SEQ ID NO: 77; PD-L1-TGF-β TRAP molecule analog—SEQ ID NO: 80/SEQ ID NO: 81; and pembrolizumab—SEQ ID NO: 78/SEQ ID NO: 79. FIG. 5A: This graph shows the induction of IFN-γ cytokine secretion by exhausted T cells of one representative donor. FIG. 5B: This graph shows the induction of IFN-γ cytokine secretion by exhausted T cells of one representative donor. FIG. 5C: This graph shows the induction of IL-2 cytokine secretion by exhausted T cells in one representative donor. FIG. 5D: This graph shows the induction of TNF-α cytokine secretion by exhausted T cells in one representative donor. FIG. 5E: This graph shows the induction of TNF-α cytokine secretion by exhausted T cells in one representative donor.

FIGS. 6A-6D—Measurement of the % inhibition of TGF-β-induced signaling induced by bispecific antibodies or control antibodies. Bispecific antibodies are: SEQ ID NO: 23×SEQ ID NO: 14; SEQ ID NO: 23×SEQ ID NO: 19; SEQ ID NO: 39×SEQ ID NO: 9; SEQ ID NO: 35×SEQ ID NO: 9; SEQ ID NO: 35×SEQ ID NO: 14; SEQ ID NO: 35×SEQ ID NO: 19; SEQ ID NO: 27×SEQ ID NO: 9; SEQ ID NO: 43×SEQ ID NO: 9; and SEQ ID NO: 43×SEQ ID NO: 19. Control antibodies are: RSV IgG1—SEQ ID NO: 86/SEQ ID NO: 87; TGF1 analog—SEQ ID NO: 76/SEQ ID NO: 77; and pembrolizumab—SEQ ID NO: 78/SEQ ID NO: 79. FIGS. 6A and 6B: These graphs show the inhibition of TGF-β signaling by bispecific antibodies or control antibodies in HEK-Blue™ TGF-β Cells. FIGS. 6C and 6D: These graphs show the inhibition of TGF-βR signaling by bispecific antibodies or control antibodies in HEK-Blue™ TGF-β-PD-1⁺ cells.

FIGS. 7A and 7B—Measurement of cytokine production induced by bispecific or control antibodies in a Treg Suppression Assay. Bispecific antibodies are: SEQ ID NO: 23×SEQ ID NO: 14; SEQ ID NO: 23×SEQ ID NO: 19; SEQ ID NO: 31×SEQ ID NO: 14; SEQ ID NO: 39×SEQ ID NO: 9; SEQ ID NO: 35×SEQ ID NO: 9; SEQ ID NO: 35×SEQ ID NO: 14; SEQ ID NO: 35×SEQ ID NO: 19; SEQ ID NO: 27×SEQ ID NO: 9; SEQ ID NO: 43×SEQ ID NO: 9; and SEQ ID NO: 43×SEQ ID NO: 19. Control antibodies are: RSV IgG1—SEQ ID NO: 86/SEQ ID NO: 87; TGF1 analog—SEQ ID NO: 76/SEQ ID NO: 77; a combination of pembrolizumab and TGF1 analog—SEQ ID NO: 78/SEQ ID NO: 79+SEQ ID NO: 76/SEQ ID NO: 77; and pembrolizumab—SEQ ID NO: 78/SEQ ID NO: 79. FIG. 7A: This graph shows the induction of IFN-γ cytokine secretion in a coculture of Tregs with PBMCs of one representative donor. FIG. 7B: This graph shows the induction of TNF-α cytokine secretion in a coculture of Tregs with PBMCs of one representative donor.

FIGS. 8A-8D—Measurement of cytokine production induced by bispecific or control antibodies in a Macrophage Suppression Assay. Bispecific antibodies are: SEQ ID NO: 35×SEQ ID NO: 9; SEQ ID NO: 23×SEQ ID NO: 14; SEQ ID NO: 23×SEQ ID NO: 19; SEQ ID NO: 43×SEQ ID NO: 9; SEQ ID NO: 39×SEQ ID NO: 9; SEQ ID NO: 43×SEQ ID NO: 19; SEQ ID NO: 35×SEQ ID NO: 14; SEQ ID NO: 35×SEQ ID NO: 19; SEQ ID NO: 31×SEQ ID NO: 14, and SEQ ID NO: 27×SEQ ID NO: 9. Control antibodies are: RSV IgG1—SEQ ID NO: 86/SEQ ID NO: 87; TGF1 analog—SEQ ID NO: 76/SEQ ID NO: 77; Opdivo; LILRB2; PD-L1-TGF-β TRAP molecule analog—SEQ ID NO: 80/SEQ ID NO: 81; pembrolizumab—SEQ ID NO: 78/SEQ ID NO: 79; and nivolumab analog—SEQ ID NO: 96/SEQ ID NO: 97. FIG. 8A: These graphs show the expression of CD163, CD209, CD206 and CD86 on M2 macrophages obtained from PBMC's from three different donors. FIGS. 8B-8D: These graphs show the induction of IFN-γ cytokine secretion by CD4⁺ T cells in the presence of M2 macrophages obtained from PBMC's from three different donors.

FIGS. 9A-9H—In vivo efficacy of bispecific antibodies. FIG. 9A: This graph shows the tumor volume reduction in mm³ induced by control and reference antibodies. FIGS. 9B-E: These graphs show the tumor volume reduction in mm³ induced by the bispecific antibodies as compared to control and reference antibodies. Bispecific antibodies are: SEQ ID NO: 43×SEQ ID NO: 9; SEQ ID NO: 43×SEQ ID NO: 19; SEQ ID NO: 23×SEQ ID NO: 14; SEQ ID NO: 23×SEQ ID NO: 19; SEQ ID NO: 39×SEQ ID NO: 9; SEQ ID NO: 27×SEQ ID NO: 9; SEQ ID NO: 23×SEQ ID NO: 18; and SEQ ID NO: 47×SEQ ID NO: 13. Control antibodies are: RSV IgG1—SEQ ID NO: 86/SEQ ID NO: 87; TGF1 analog—SEQ ID NO: 76/SEQ ID NO: 77; pembrolizumab—SEQ ID NO: 78/SEQ ID NO: 79; PD-L1-TGF-β TRAP molecule analog—SEQ ID NO: 80/SEQ ID NO: 81; and a combination of pembrolizumab and TGF1 analog—SEQ ID NO: 78/SEQ ID NO: 79+SEQ ID NO: 76/SEQ ID NO: 77. Bispecific antibodies were dosed at 1 mg/kg (1) and/or 10 mg/kg (10) (FIGS. 9A-9C) and at 10 mg/kg only (FIGS. 9D-9F). FIG. 9G: This graph shows the TGF-βRII receptor occupancy. FIG. 9H: This graph shows the PD-1 receptor occupancy after treatment with the bispecific or control antibodies.

FIG. 10—Vector map

FIG. 11—In vivo efficacy of bispecific antibodies. This graph shows the tumor volume reduction in mm³ induced by an exemplary bispecific antibody at two different dose levels: 1 mg/kg and 10 mg/kg, as compared to a control antibody at 10 mg/kg. The bispecific antibody is: SEQ ID NO: 23×SEQ ID NO: 18, and the control antibody is: SEQ ID NO: 86/SEQ ID NO: 87.

### EXAMPLES

In the Examples, which are used to illustrate the present disclosure but are not intended to limit the disclosure in any way, each binding domain of the bispecific antibodies comprises a light chain variable region variable region having an amino acid sequence as set forth in SEQ ID NO: 48 and a light chain constant region having an amino acid sequence as set forth in SEQ ID NO: 75. The bispecific antibodies preferably are IgG1 antibodies comprising a CH1, hinge, CH2, and CH3. In the Examples, which are used to illustrate the present disclosure but are not intended to limit the disclosure in any way, bispecific antibodies were screened in IgG1 format, wherein the PD-1 binding heavy chain comprises a CH1 having an amino acid sequence as set forth in SEQ ID NO: 69, a CH2 having an amino acid sequence as set forth in SEQ ID NO: 71, and a CH3 having an amino acid sequence as set forth in SEQ ID NO: 73; and the TGF-βRII binding heavy chain comprises a CH1 having an amino acid sequence as set forth in SEQ ID NO: 69, a CH2 having an amino acid sequence as set forth in SEQ ID NO: 71, and a CH3 having an amino acid sequence as set forth in SEQ ID NO: 74.

Reference antibodies and molecules, and control antibodies used in the Examples include:
Reference PD-1 antibody pembrolizumab analog, which is a bivalent monospecific antibody comprising two heavy chains having an amino acid sequence as set forth in SEQ ID NO: 78 and two light chains having an amino acid sequence as set forth in SEQ ID NO: 79.
Reference PD-1 antibody pembrolizumab (made by Merck, distributed by Myonex).
Reference TGF-βRII antibody TGF1, which is a bivalent monospecific analog of TGF1 and comprises two heavy chains having an amino acid sequence as set forth in SEQ ID NO: 76 and two light chains having an amino acid sequence as set forth in SEQ ID NO: 77.
Reference PD-L1-TGF-β TRAP molecule, which is an analog of Bintrafusp alfa bivalent monospecific for binding to PD-L1 and comprises two heavy chains having an amino acid sequence as set forth in SEQ ID NO: 80 and two light chains having an amino acid sequence as set forth in SEQ ID NO: 81, with an extracellular domain of TGF-βRII having an amino acid sequence as set forth in SEQ ID NO: 104 linked to the C-terminus of each heavy chain through a (G₄S)₄G linker.
Negative control IgG1 antibody (RSV-G), which is a bivalent monospecific antibody comprising two heavy chains having an amino acid sequence as set forth in SEQ ID NO: 86 and two light chains having an amino acid sequence as set forth in SEQ ID NO: 87.
Control TGF-βRIIxRSV antibodies, which are bivalent bispecific antibodies comprising a TGF-βRII binding domain comprising a heavy chain variable region amino acid sequence as set forth in SEQ ID NO: 23, 31, 39, 27, 35, or 43; a RSV binding domain comprising a heavy chain variable region amino acid sequence as set forth in SEQ ID NO: 86; and a common light chain comprising a light chain variable region amino acid sequence as set forth in SEQ ID NO: 48 and a light chain constant region amino acid sequence as set forth in SEQ ID NO: 75.
Positive control LILRB2 (BioLegend; Cat. No. 338714).
Commercial reference PD-1 antibody Opdivo (made by Bristol Myers Squibb (BMS).

### Example 1—Generation of PD-1xTGF-βRII Bispecific Antibodies

Binding domains, antibodies and heavy chain variable regions with binding specificity to human PD-1 and heavy chain variable regions with binding specificity to human TGF-βRII were obtained by immunizing transgenic mice comprising a common IGKV1-39 light chain (MeMo® mice) with human PD-1 or TGF-βRII antigenic moieties, including the use of different forms of DNA, protein and cell-based antigen delivery.

Heavy chain variable regions with binding specificity to human PD-1 having an amino acid sequence as set forth in SEQ ID NO: 1; 5; 9; 13; 14; 18; and 19, and heavy chain variable regions with binding specificity to human TGF-βRII having an amino acid sequence as set forth in SEQ ID NO: 23; 27; 31; 35; 39; 43; 47; 88; and 89, were selected for the production of bispecific antibodies. The binding domain sequences herein, once characterized and sequenced through the techniques provided herein, can be subsequently obtained by any method known in the art.

TABLE 1
Combination ofPD-1 heavy chain variable regions and TGF-/3R11 heavy
chain variable regions that can be used in the generation of bispecific antibodies.

| | TGF-βRII | | | | | | | | | |
|---|---|---|---|---|---|---|---|---|---|---|
| | | SEQ ID | SEQ ID | SEQ ID | SEQ ID | SEQ ID | SEQ ID | SEQ ID | SEQ ID | SEQ ID |
| PD-1 | | NO: 23 | NO: 27 | NO: 31 | NO: 35 | NO: 39 | NO: 43 | NO: 47 | NO: 88 | NO: 89 |
| | SEQ ID | PBI | PB2 | PB3 | PB4 | PB5 | PB6 | PB7 | PB50 | PB57 |
| | NO: 1 | | | | | | | | | |
| | SEQ ID | PB8 | PB9 | PB10 | PB11 | PB12 | PB13 | PB14 | PB51 | PB58 |
| | NO: 5 | | | | | | | | | |
| | SEQ ID | PB15 | PB16 | PB17 | PB18 | PB19 | PB20 | PB21 | PB52 | PB59 |
| | NO: 9 | | | | | | | | | |
| | SEQ ID | PB22 | PB23 | PB24 | PB25 | PB26 | PB27 | PB28 | PB53 | PB60 |
| | NO: 13 | | | | | | | | | |
| | SEQ ID | PB29 | PB30 | PB31 | PB32 | PB33 | PB34 | PB35 | PB54 | PB61 |
| | NO: 14 | | | | | | | | | |
| | SEQ ID | PB36 | PB37 | PB38 | PB39 | PB40 | PB41 | PB42 | PB55 | PB62 |
| | NO: 18 | | | | | | | | | |
| | SEQ ID | PB43 | PB44 | PB45 | PB46 | PB47 | PB48 | PB49 | PB56 | PB63 |
| | NO: 19 | | | | | | | | | |

Bispecific IgG antibodies were generated by transient co-transfection of two plasmid vectors: one encoding an IgG heavy chain with a PD-1 binding VH region and the other encoding an IgG heavy chain with a TGF-βRII binding VH region. CH3 engineering technology as described in WO 2013/157954 and WO 2013/157953 was employed to ensure efficient hetero-dimerization and formation of bispecific antibodies. Both vectors further encode a common light chain comprising the IGKV1-39/Jk1 light chain variable region. Cell transfection, cell culture, and the harvesting and purification of antibodies was performed by methods known in the art.

### Example 2—PD-1-SHP Recruitment Assay

Bispecific antibodies were characterized in a PD-1-SHP recruitment assay to determine their ability to block ligand binding to PD-1 and thereby inhibit PD-1/PD-L1 signaling in T cells. A PD-1-SHP recruitment assay involves a two cell system comprising U2OS cells engineered to express an Enzyme Donor (ED)-tagged PD-1 receptor (e.g. PD-L1 or PD-L2) and Jurkat T cells expressing PD-1 and engineered to express Enzyme Acceptor (EA)-fused SHP1. Ligand or agonistic antibody-induced PD-1 activation of the Jurkat T cells results in SHP1 recruitment, forcing ED and EA interaction and of reconstitution of an active β-gal enzyme. Reconstituted β-gal produces a chemiluminescent signal in the presence of substrate.

Antibody samples included several bispecific antibodies, negative control IgG1 antibody (RSV), reference PD-1 antibody pembrolizumab analog, an analog of reference TGF-βRII antibody TGF1, and an analog of reference PD-L1-TGF-β TRAP molecule.

U2OS/PD-L1 cells (DiscoveRx Corporation) were maintained in McCoy's 5A medium (Thermo Fisher Scientific) with addition of 10% FBS+0.25 μm/ml Puromycin (Thermo Fisher Scientific). Jurkat-PD-1-SHP cells (Src homology region 2 domain-containing phosphatase; DiscoveRx Corporation) were cultured in RPMI1640 medium (Thermo Fisher Scientific) supplemented with 10% FBS, 250 μg/ml Hygromycin B (Thermo Fisher Scientific), and 500 μg/ml G418 (Thermo Fisher Scientific). Both U2OS/PD-L1 and Jurkat-PD-1-SHP cells were first centrifuged in a conical tube to remove the culture media, and then washed, and resuspended with assay medium (RPMI1640 medium with 1% FBS) before cell plating. The U2OS/PD-L1 cells were added in a 384-well black clear bottom assay plate (CELLCOAT® Tissue Culture Plates, Greiner Bio-One) at 5000 cells per well in 20 μL assay medium. Antibody samples were prepared by serial dilution in phosphate buffered saline (PBS) with 1% FBS, and 5 μL/well was transferred to the cell plate and incubated at 37° C., 5% CO₂ for one hour. Jurkat-PD-1-SHP cells were subsequently added to the cell plate at 5000 cells per well in 20 μL assay medium and incubated at 37° C., 5% CO₂ for two hours before the addition of 2.5 μL PathHunter reagent 1 (DiscoveRx Corporation) in each well. The assay plate was then shaken for 1 min at 350 rpm and kept in the dark for 15 minutes at room temperature followed by addition of 10 μL PathHunter reagent 2 (DiscoveRx Corporation). Chemiluminescent signal was recorded with TopCount reader (Perkin Elmer) after incubation at room temperature for one hour. Wells with PBS only served as the positive controls and wells containing no cells were used as negative controls. IC₅₀ determination was performed by fitting the curve of percent control activity versus the log of the compound concentration using the GraphPad Prism 7.0 software.

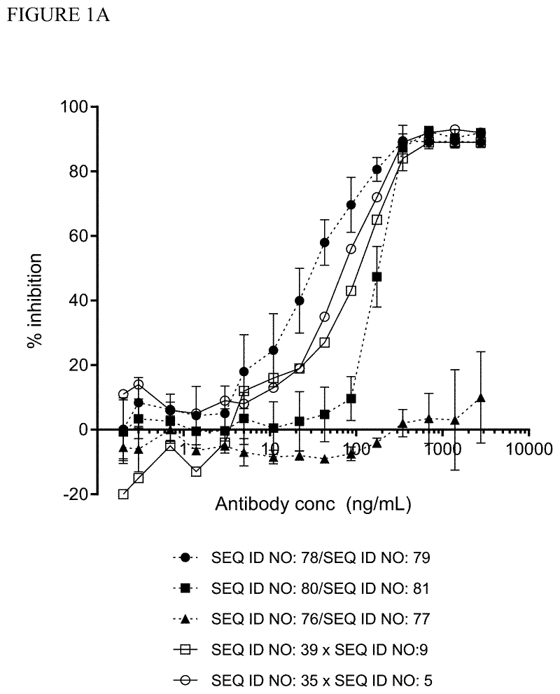

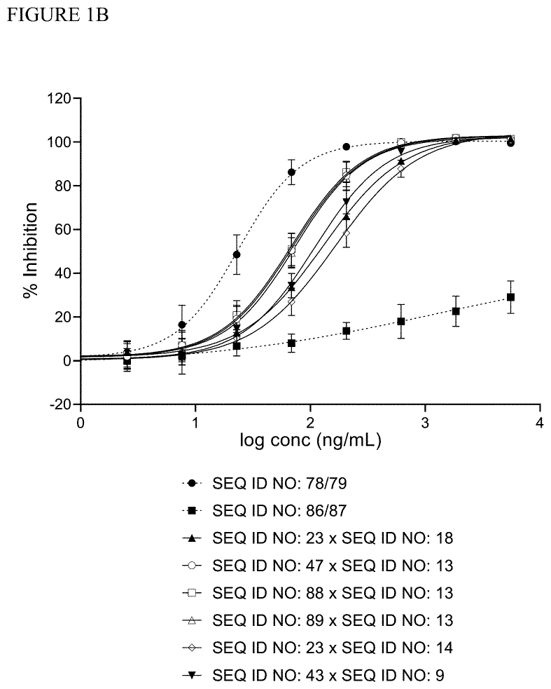

Results are shown in Table 2 and FIGS. 1A and 1B. All bispecific antibodies inhibited PD-1-mediated SHP recruitment. A number of bispecific antibodies, that are monovalent for PD-1 binding, are equipotent to bivalent monospecific reference PD-1 antibody pembrolizumab analog and reference PD-L1-TGF-β TRAP molecule, which is bivalent for PD-L1 binding, in the PD-1-SHP Recruitment Assay.

TABLE 2
Results of the PD-1-SHP Recruitment Assay.

| | SEQ ID | SEQ ID | SEQ ID | SEQ ID | SEQ ID | SEQ ID | SEQ ID |
|---|---|---|---|---|---|---|---|
| IC₅₀(ng/mL) | NO: 86 | NO: 23 | NO: 31 | NO: 39 | NO: 27 | NO: 35 | NO: 43 |
| SEQ ID NO: 86 | — | — | — | — | — | — | — |
| SEQ ID NO: 1 | 271 | 277 | 390 | 428 | 410 | 415 | 278 |
| SEQ ID NO: 5 | 126 | 141 | 160 | 158 | 118 | 78 | 312 |
| SEQ ID NO: 9 | 101 | 120 | 137 | 94 | 122 | 130 | 226 |
| SEQ ID NO: 14 | 202 | 257 | 337 | 251 | 167 | 255 | 238 |
| SEQ ID NO: 19 | 207 | 234 | 400 | 237 | 232 | 220 | 267 |

### Example 3—PD-1-NFAT Reporter Assay

Bispecific antibodies were characterized in a PD-1-NFAT Reporter Assay to determine their ability to block PD-1/PD-L1 signaling in activated T cells. A PD-1-NFAT Reporter Assay involves a two transgenic cell line system with PD-L1⁺ aAPC/CHO-K1 cells co-expressing a TCR cognate protein and PD-1⁺ effector Jurkat T cells driving luciferase reporter under control of a NFAT-RE cis element. Effector Jurkat T cells activate TCR signaling in an antigen-independent manner. PD-1/PD-L1 interaction inhibits TCR-mediated luminescence. Blockade of the PD-1/PD-L1 interaction allows TCR signaling, thereby inducing luminescence detectable by adding substrate.

Antibody samples included several bispecific antibodies, negative control IgG1 antibody (RSV), reference PD-1 antibody pembrolizumab analog, an analog of reference TGF-βRII antibody TGF1, and an analog of reference PD-L1-TGF-β TRAP molecule.

PD-L1 aAPC/CHO-K1 cells (artificial Antigen Presenting Cell)/CHO (Chinese Hamster Ovary)-K1 cells; Promega) were maintained in F-12 medium (Thermo Fisher Scientific) with addition of 10% FBS, 200 μg/mL Hygromycin B (Thermo Fisher Scientific), and 250 μg/mL Geneticin (G418; Thermo Fisher Scientific). Jurkat-PD-1-NFAT effector cells (Nuclear Factor of Activated T cells; Promega) were cultured in RPMI 1640 medium (Thermo Fisher Scientific) supplemented with 10% FBS, 100 μg/mL Hygromycin B (Thermo Fisher Scientific), and 500 μg/mL G418 (Thermo Fisher Scientific). Both PD-L1 aAPC/CHO-K1 cells and Jurkat-PD NFAT effector cells were centrifuged first to remove the culture media, then washed and resuspended with assay medium (RPMI1640 medium with 1% FBS) before cell plating. The PD-L1 aAPC/CHO-K1 cells were added to a 384-well white clear-bottom assay plate (CELLCOAT® Tissue Culture Plates, Greiner Bio-One) at 8000 cells per well in 10 μL assay medium. Antibody samples were prepared by serial dilution in phosphate buffered saline (PBS) with 1% FBS and 5 μL/well was transferred to the cell plate. Jurkat-PD-1-NFAT effector cells were then dispensed into each well at 10,000 cells per well in 5 μL assay medium. The assay plate was incubated at 37° C., 5% CO₂ for 24 hours. After the assay plate was equilibrated to room temperature for 15 minutes, 20 μL/well of Bio-Glo™ reagent (Promega) was added. After 8 minutes of incubation at room temperature, luminescence was read out with a Pherastar microplate reader (BMG Labtech). Wells with PBS served as the negative controls (0% induction) and wells containing 12.5 ug/mL pembrolizumab analog were used as positive controls (100% induction). EC₅₀ determination was performed by fitting the curve of percent control activity versus the log of the compound concentration using the GraphPad Prism 7.0 software.

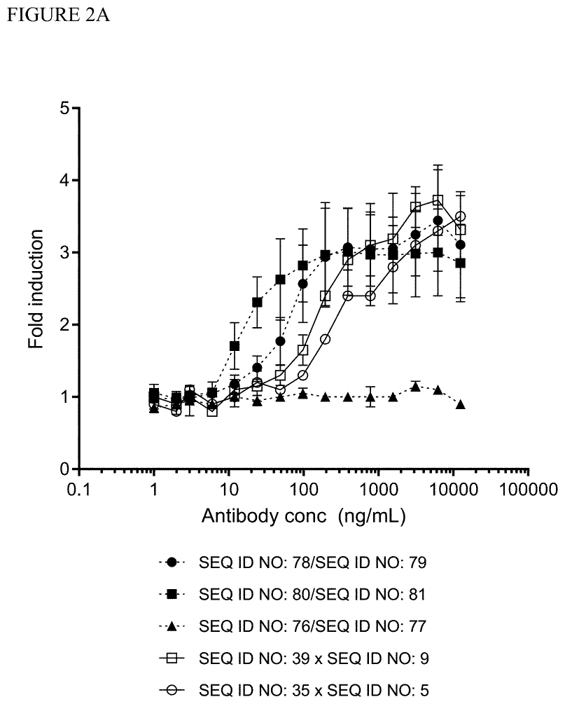

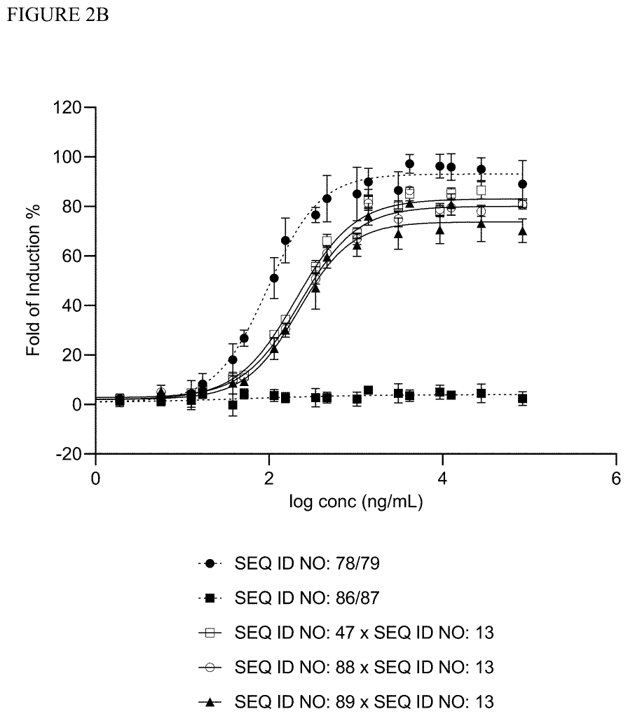

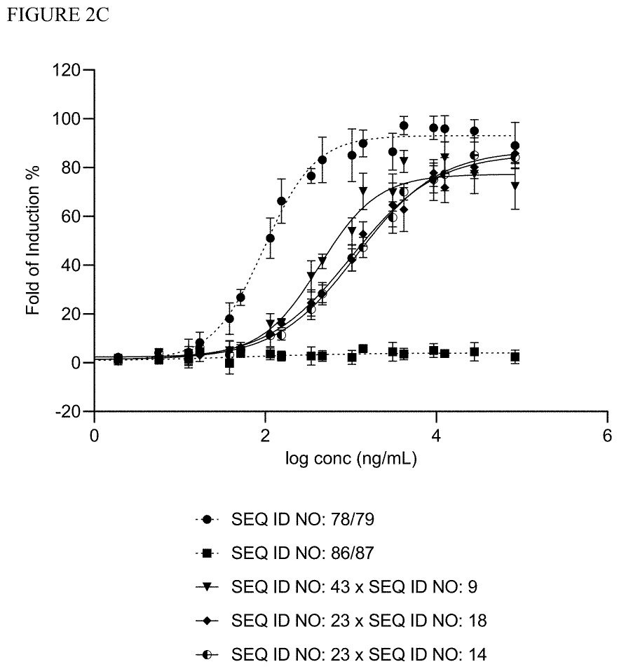

Results are shown in Table 3 and FIGS. 2A-2C. All bispecific antibodies inhibited PD-1-mediated T cell inhibition. A number of bispecific antibodies, that are monovalent for PD-1 binding, are equipotent to bivalent monospecific reference PD-1 antibody pembrolizumab analog in the PD-1-NFAT Reporter Assay.

TABLE 3
Results of the PD-1-NFAT Reporter Assay.

| EC₅₀ | SEQ ID | SEQ ID | SEQ ID | SEQ ID | SEQ ID | SEQ ID | SEQ ID |
|---|---|---|---|---|---|---|---|
| (ng/mL) | NO: 86 | NO: 23 | NO: 31 | NO: 39 | NO: 27 | NO: 35 | NO: 43 |
| SEQ ID NO: 86 | — | — | — | — | — | — | — |
| SEQ ID NO: 1 | 1352 | 1828 | 3569 | 1775 | 756 | 2030 | 1236 |
| SEQ ID NO: 5 | 545 | 444 | 535 | 299 | 369 | 533 | 555 |
| SEQ ID NO: 9 | 346 | 448 | 325 | 287 | 317 | 664 | 257 |
| SEQ ID NO: 14 | 1513 | 869 | 868 | 904 | 772 | 686 | 790 |
| SEQID NO: 19 | 791 | 771 | 2900 | 1019 | 741 | 936 | 681 |

### Example 4—Jurkat phosphoSMAD2/3 Assay

Bispecific antibodies were characterized in a Jurkat phosphoSMAD2/3 Assay to determine their ability to block TGF-βRII signaling in T cells. The Jurkat phosphoSMAD2/3 Assay involved a comparison of the activity of the bispecific antibodies on Jurkat-PD-1^{null} cells and Jurkat-PD-1⁺ cells to determine if the bispecific antibodies inhibit TGF-β-induced SMAD2/3 phosphorylation in a PD-1 correlated manner. An analog of reference TGF-βRII antibody TGF1, a TGF-βRIIxRSV bispecific antibody, and a negative control bivalent monospecific IgG1 antibody comprising a heavy chain having an amino acid sequence as set forth in SEQ ID NO: 86 and a light chain having an amino acid sequence as set forth in SEQ ID NO: 87 were included as control antibodies.

Jurkat-PD-1^{null} (ATCC Cat. no. TIB-152) and Jurkat-PD-1⁺ cells (Promega Cat. no. CS187105) in RPMI/10% FBS were seeded in 96-well flat-bottom plates. PD-1 expression was undetectable for the Jurkat-PD-1^{null} cells; the number of PD-1 molecules on the Jurkat-PD-1⁺ cells was determined at about 4000, using quantibrite bead methodology. Bispecific and control antibodies were added in 6-step serial dilutions (100 μg/ml to 0.001 μg/ml), and the cells were incubated for one hour at 37° C./5% CO₂. After one hour, human recombinant TGF-β1 (R&D Systems Cat. no. 7754-BH) was added at a final concentration of 10 ng/ml and the cells were incubated for two more hours at 37° C./5% CO₂. After incubation, cells were washed gently with PBS. Cell lysates were prepared using lysis buffer (MSD #R60TX-2) containing phosphatase inhibitors (Sigma #P0044 and P-5726) and protease inhibitors (Pierce Biotechnology #87785). Samples were normalized to total protein using BCA protein assay kit (Pierce #23227). PhosphoSMAD2/3 levels were determined using ELISA (Cell Signaling #12001) according to manufacturer's instructions. GraphPad Prism (8.2.0) was used to plot the graphs.

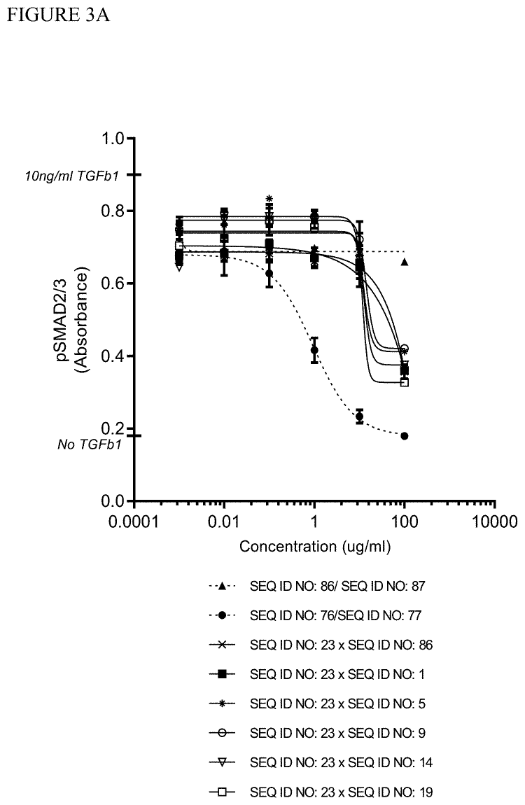

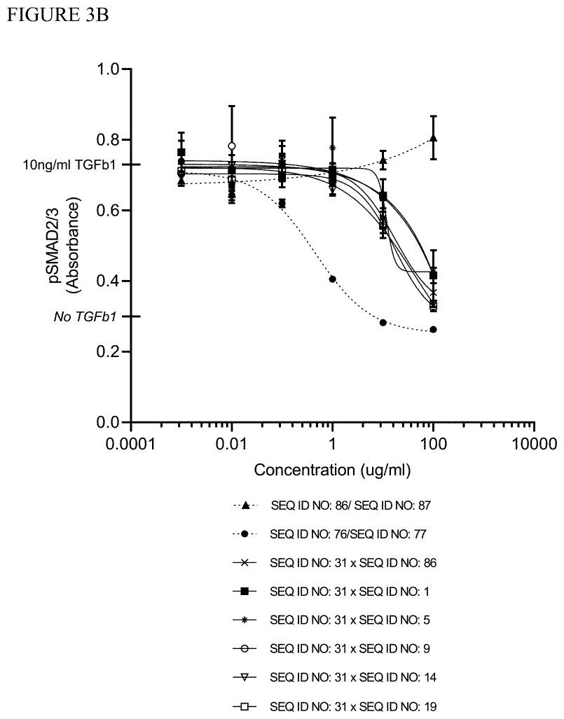

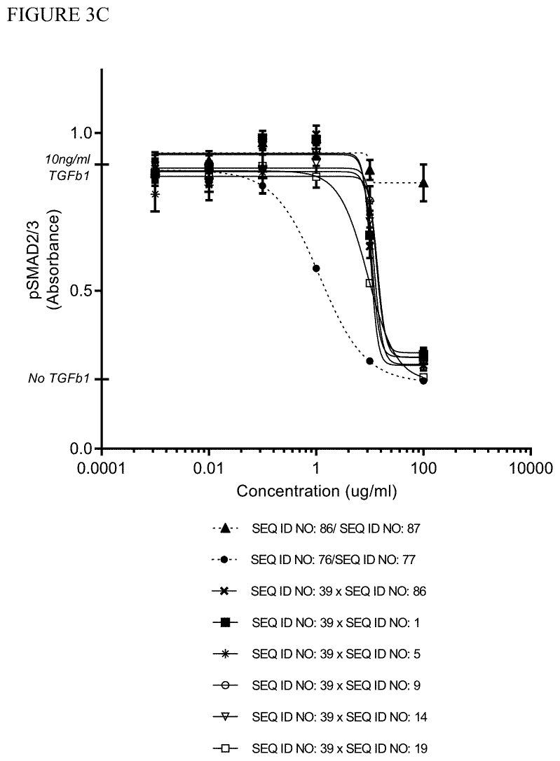

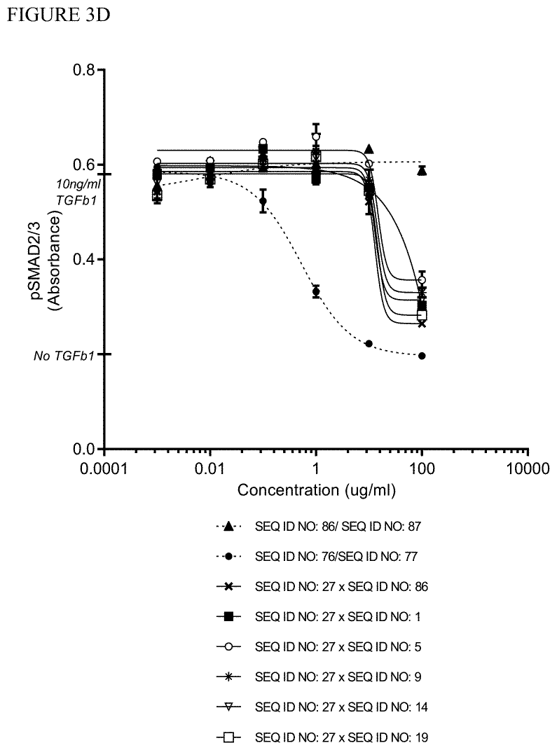

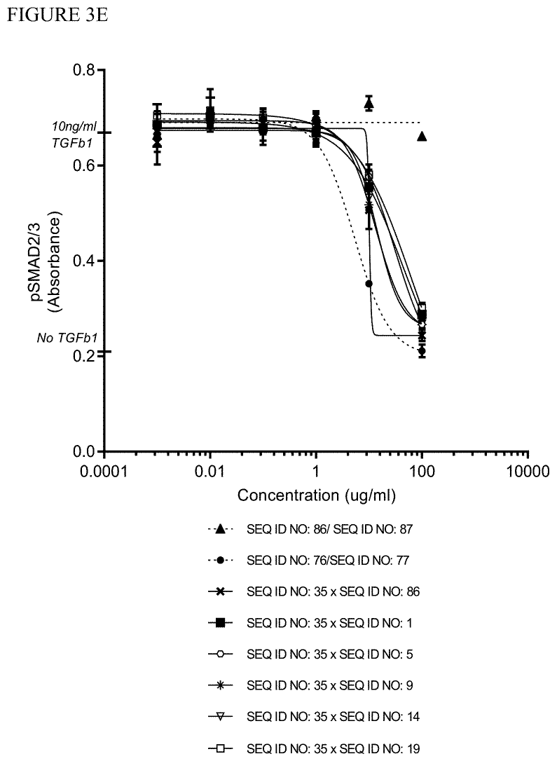

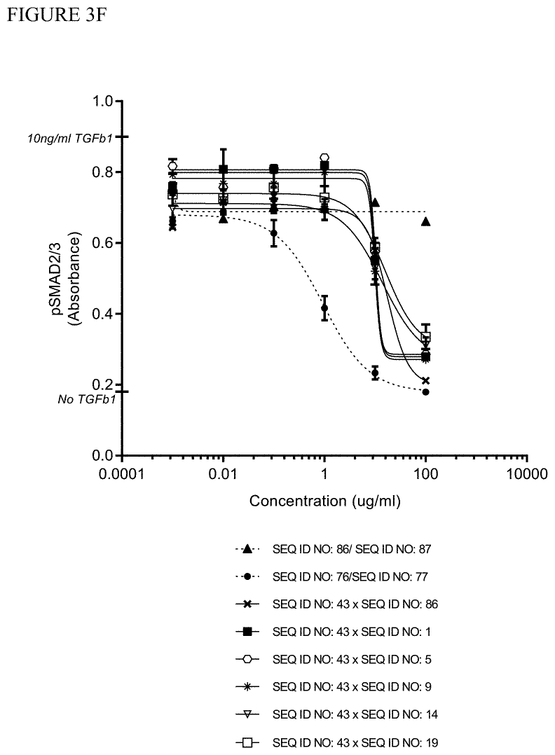

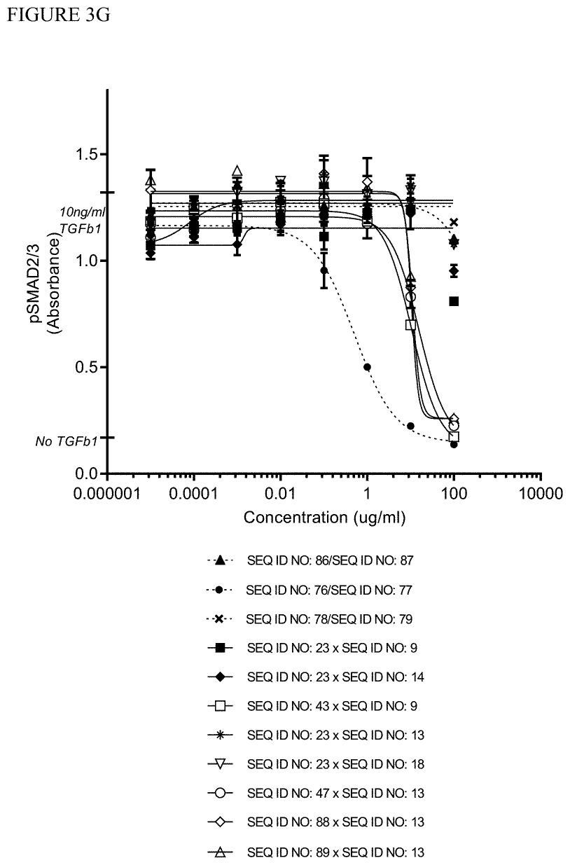

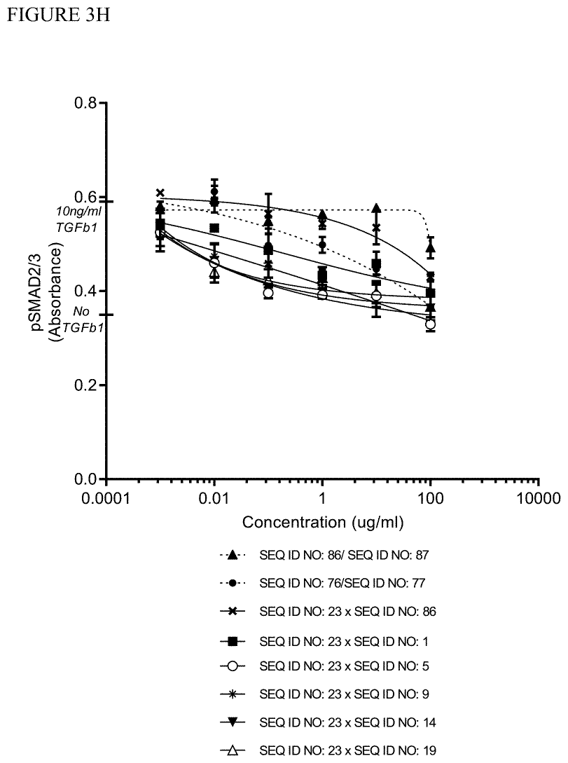

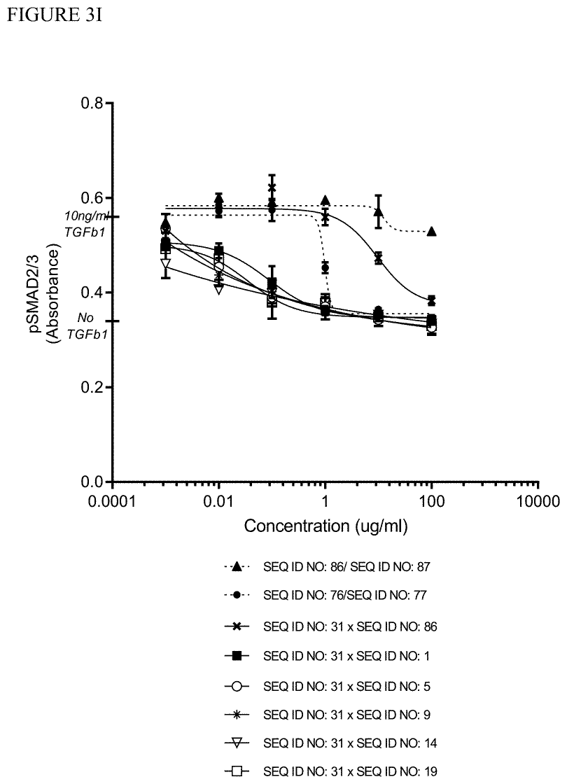

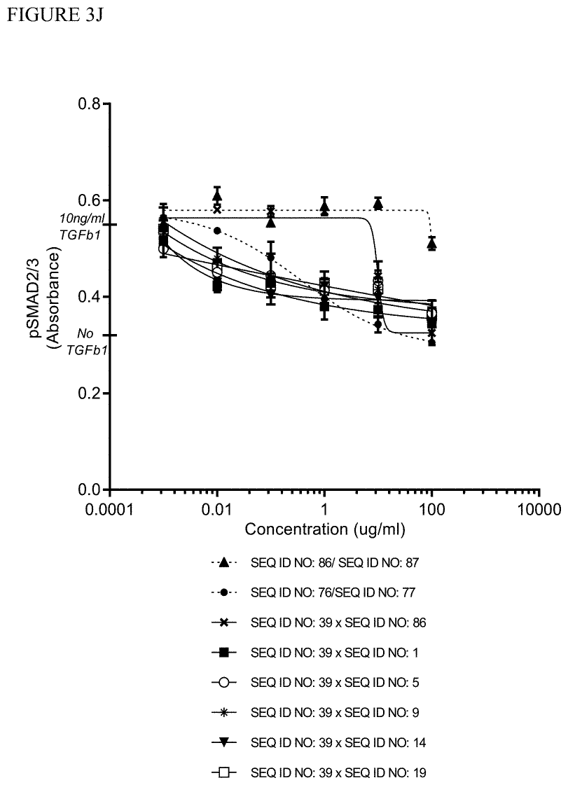

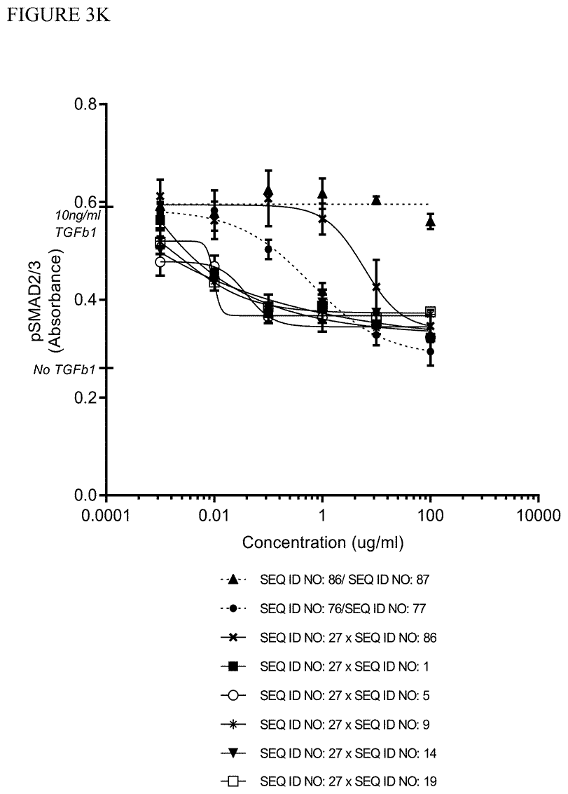

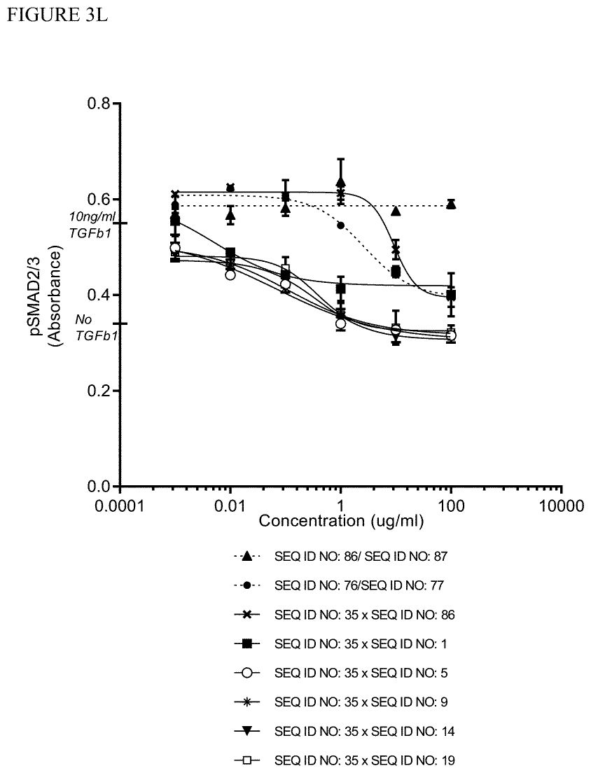

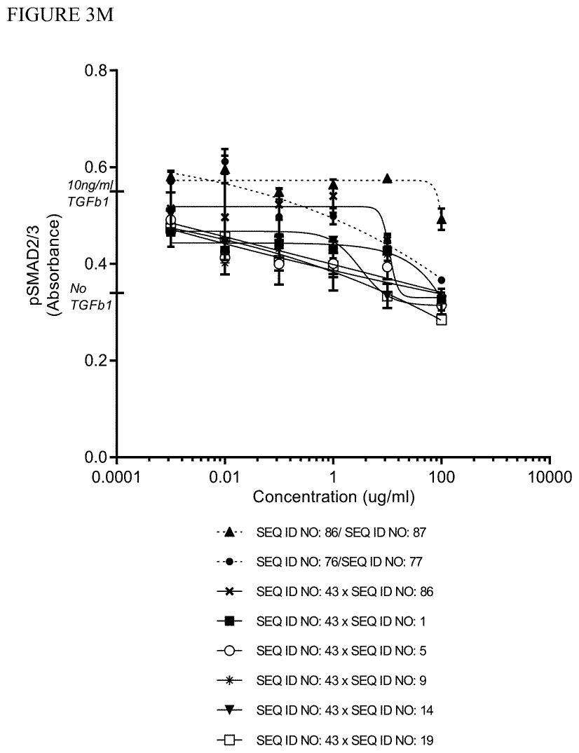

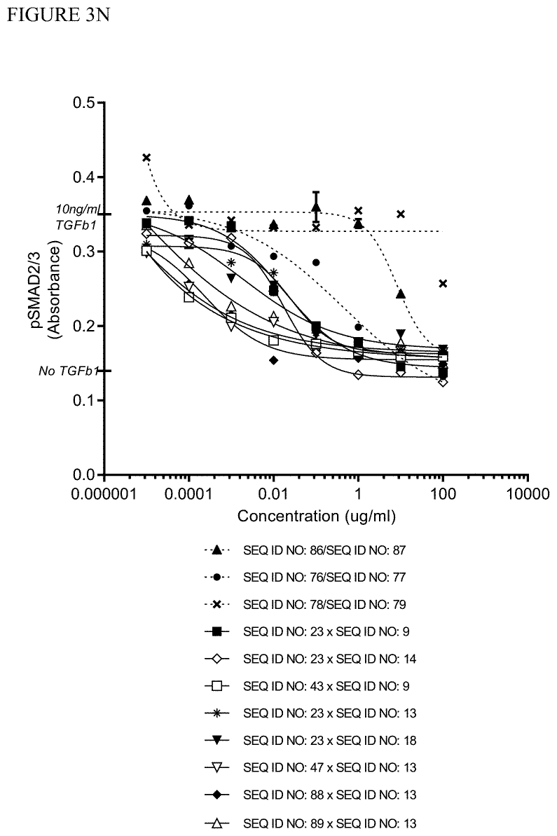

Results are shown in Tables 4, 5 and 6, and FIGS. 3A-3N. Table 6 shows the fold difference in potency in inhibiting TGF-βRII signaling in Jurkat-PD-1^{null} versus Jurkat-PD-1⁺ cells of 10 bispecific antibodies. The fold difference of the bispecific antibodies is between 200-11000 fold higher than that of the analog of reference antibody TGF1 in this assay.

All bispecific antibodies inhibited TGF-βRII signaling in both Jurkat-PD-1^{null} cells and Jurkat-PD-1⁺ cells. The bispecific antibodies are more potent in inhibiting TGF-βRII signaling in Jurkat-PD-1⁺ cells than in Jurkat-PD-1^{null} cells, indicating that they inhibit TGF-β-induced SMAD2/3 phosphorylation in a manner correlated to PD-1 expression. All bispecific antibodies require a higher concentration to inhibit TGF-βRII signaling in Jurkat-PD-1^{null} cells than the analog of reference TGF-βRII antibody TGF1. Many bispecific antibodies, that are monovalent for binding to TGF-βRII, are superior to the bivalent monospecific analog of reference TGF-βRII antibody TGF1 in inhibiting TGF-βRII signaling in Jurkat-PD-1⁺ cells.

TABLE 4
IC₅₀ values of the Jurkat phosphoSMAD2/3 Assay in Jurkat-PD-1^{null} cells.

| | SEQ ID | SEQ ID | SEQ ID | SEQ ID | SEQ ID | SEQ ID | SEQ ID |
|---|---|---|---|---|---|---|---|
| | NO: 86 | NO: 23 | NO: 31 | NO: 39 | NO: 27 | NO: 35 | NO: 43 |
| SEQ ID | — | 54560 | 19170 | 14190 | 31510 | 15700 | 21600 |
| NO: 86 | | | | | | | |
| SEQ ID | — | 60540 | 38700 | 17200 | 42960 | 23030 | 15240 |
| NO: 1 | | | | | | | |
| SEQ ID | — | 41320 | 43600 | 30640 | 64230 | 24590 | 18080 |
| NO: 5 | | | | | | | |
| SEQ ID | — | 70920 | 32920 | 29670 | 55380 | 16400 | 13100 |
| NO: 9 | | | | | | | |
| SEQ ID | — | 55380 | 13620 | 21750 | 46580 | 19850 | 15380 |
| NO: 14 | | | | | | | |
| SEQ ID | — | 41450 | 15250 | 8383 | 40960 | 26050 | 22760 |
| NO: 19 | | | | | | | |

TABLE 5
IC₅₀ values of the Jurkat phosphoSMAD2/3 Assay in Jurkat-PD-1⁺ cells.

| IC₅₀ | SEQ ID | SEQ ID | SEQ ID | SEQ ID | SEQ ID | SEQ ID | SEQ ID |
|---|---|---|---|---|---|---|---|
| (ng/mL) | NO: 86 | NO: 23 | NO: 31 | NO: 39 | NO: 27 | NO: 35 | NO: 43 |
| SEQ ID | — | 37100 | 18160 | 10010 | 6118 | 31580 | 10130 |
| NO: 86 | | | | | | | |
| SEQ ID | — | 182 | 27 | 9 | 7 | 577 | 16 |
| NO: 1 | | | | | | | |
| SEQ ID | — | 3 | 15 | 24 | 35 | 10 | 7 |
| NO: 5 | | | | | | | |
| SEQ ID | — | 128 | 10 | 101 | 7 | 13 | 17 |
| NO: 9 | | | | | | | |
| SEQ ID | — | 4 | 1 | 10 | 10 | 11 | 53 |
| NO: 14 | | | | | | | |
| SEQ ID | — | 3 | 11 | 56 | 6 | 21 | 11 |
| NO: 19 | | | | | | | |

TABLE 6
Fold difference in potency in inhibiting TGF-βRII
signaling in Jurkat-PD-1^{null} versus Jurkat-PD-1⁺ cells.

| | | Fold difference pSMAD |
|---|---|---|
| TGF-βRII | PD-1 | PD-1^{null} vs PD-1⁺ |
| SEQ ID NO: 23 | SEQ ID NO: 14 | 13845 |
| SEQ ID NO: 23 | SEQ ID NO: 19 | 13817 |
| SEQ ID NO: 31 | SEQ ID NO: 14 | 13620 |
| SEQ ID NO: 39 | SEQ ID NO: 9 | 294 |
| SEQ ID NO: 35 | SEQ ID NO: 9 | 1262 |
| SEQ ID NO: 35 | SEQ ID NO: 14 | 1805 |
| SEQ ID NO: 35 | SEQ ID NO: 19 | 1240 |
| SEQ ID NO: 27 | SEQ ID NO: 9 | 7911 |
| SEQ ID NO: 43 | SEQ ID NO: 9 | 771 |
| SEQ ID NO: 43 | SEQ ID NO: 19 | 2069 |
| | SEQ ID NO: 78/ | NA |
| | SEQ ID NO: 79 | |
| SEQ ID NO: | | 1.3 |
| 76/SEQ ID | | |
| NO: 77 | | |

### Example 5—phosphoSMAD2 Assay with Stimulated and Unstimulated CD4⁺ and CD8⁺ T Cells

Bispecific antibodies were characterized in a phosphoSMAD2 assay to determine their specificity in blocking TGF-βRII signaling in PD-1 positive T cells. The phosphoSMAD2 assay involved a comparison of the activity of the bispecific antibodies on unstimulated T cells, expressing low levels of PD-1, and stimulated T cells, expressing high levels of PD-1, to determine if the bispecific antibodies inhibit TGF-β-induced SMAD2 phosphorylation in a manner correlated to PD-1 expression. An analog of reference TGF-βRII antibody TGF1, reference PD-1 antibody pembrolizumab, and a negative control bivalent monospecific IgG1 antibody comprising a heavy chain having an amino acid sequence as set forth in SEQ ID NO: 86 and a light chain having an amino acid sequence as set forth in SEQ ID NO: 87, were included as positive controls and a negative control, respectively.

PBMCs from healthy donors were stimulated with 1 μg/ml anti-CD3 antibody (BD Biosciences #555336) for 48 hours followed by 16 hour serum deprivation (0.1% FBS). The number of PD-1 molecules on activated CD4⁺ and CD8⁺ T cells was determined at about 1000-2000, using quantibrite bead methodology. Stimulated and unstimulated PBMCs were then incubated with bispecific and control antibodies for 30 min at room temperature. Recombinant human TGF-β1 (Cell Signaling #7754-BH) was added at a final concentration of 1 ng/ml and the cells were incubated for another 30 min. Finally, cells were washed twice with PBS and stained for cell surface markers followed by intracellular phosphoSMAD2 staining.

The following antibodies were used in staining for flow cytometry: antibodies against human CD45 (clone: HI30; cat557748, BD Biosciences), human CD11b (clone: M1/70; cat #563015, BD Biosciences), human CD3 (Clone: UCHT1; cat #565491, BD Biosciences), human CD4 (Clone: SK3; cat #563550, BD Biosciences), human CD8 (clone #SK1, cat #344714, Biolegend) and human phospho-SMAD2 (cat #56532, Cell Signaling). Viability dye (Biolegend #423114) was used to exclude dead cells during analysis. Cell acquisition was performed under FACSymphony A3 using DIVA software.

PBMCs were gated based on size and granularity using FSC-A vs SSC-A to exclude debris. Dead cells were then excluded using fixable viability dye. CD45 positive cells were selected followed by a CD11b negative and CD3 positive T cell selection. Finally, CD4 and CD8 positive subsets were gated and phospho-SMAD2 signal was measured in geo mean fluorescence intensity (GMFI) on these subsets. Data analysis was performed using FlowJo software and GrapPad Prism (8.2.0) was used to plot the graphs.

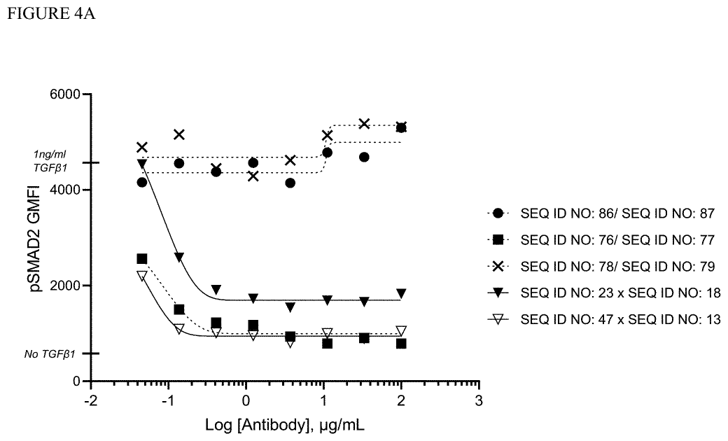

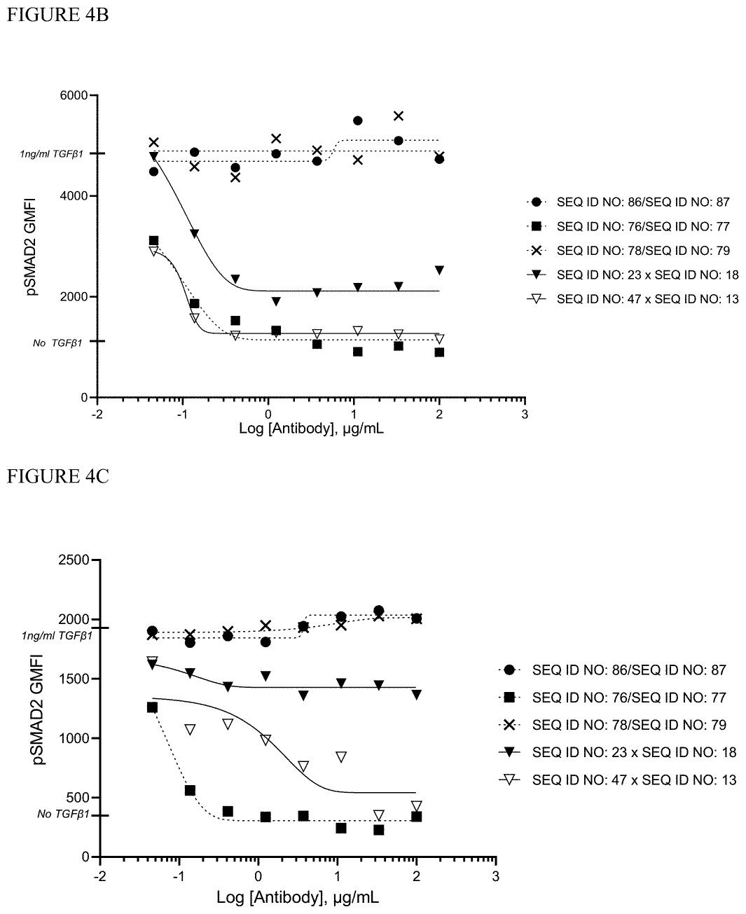

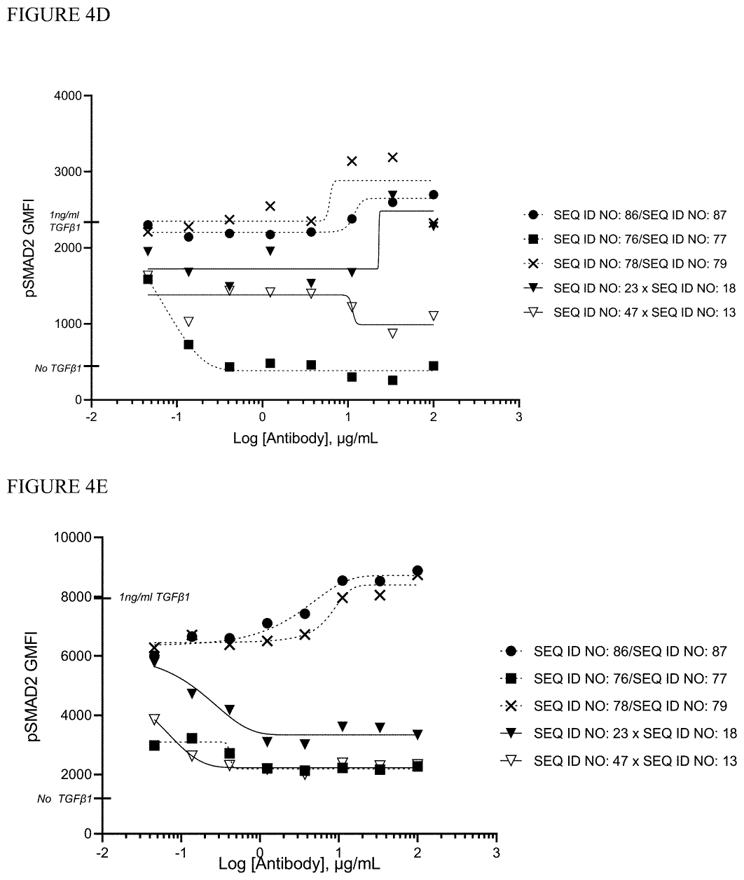

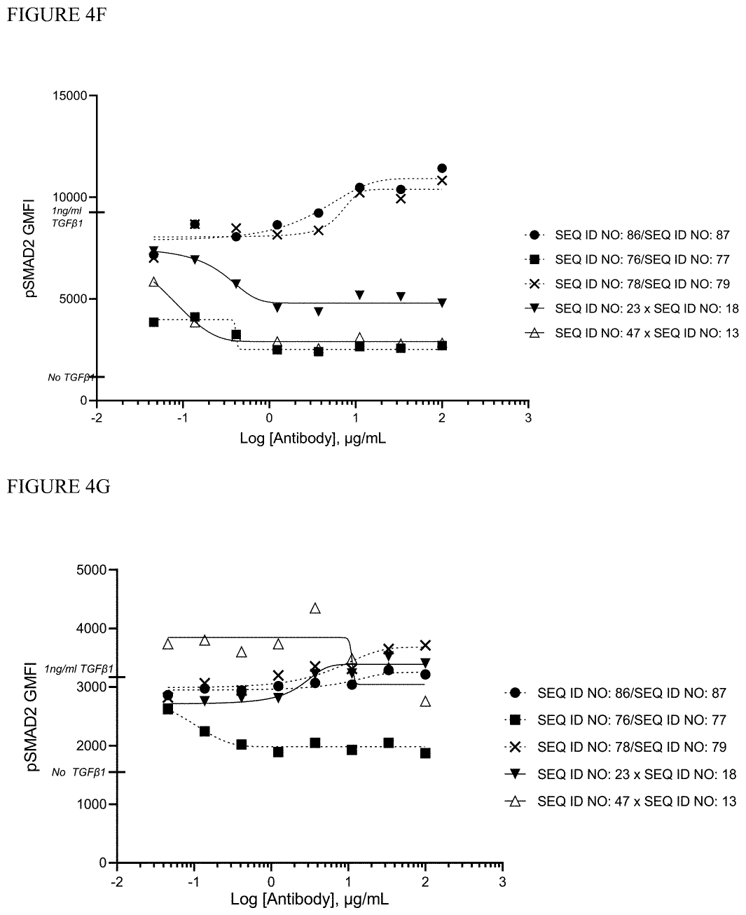

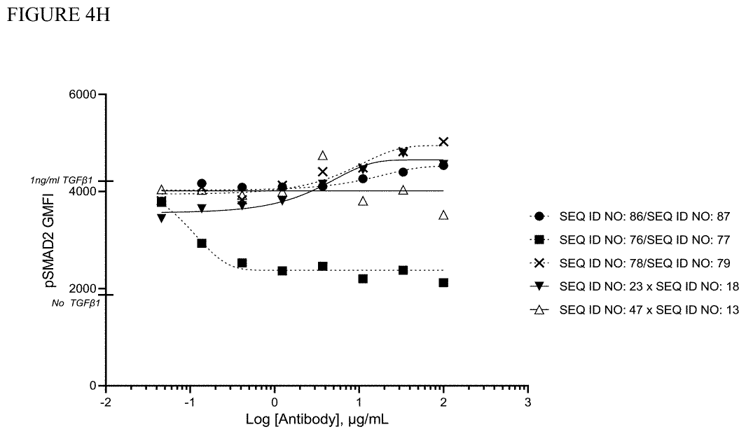

Results are shown in FIGS. 4A-4H. All bispecific antibodies inhibited TGF-βRII signaling in stimulated CD4⁺ and CD8⁺ T cells. In one donor (donor B), the bispecific antibodies did not inhibit TGF-βRII signaling in unstimulated CD4⁺ and CD8⁺ T cells. In the other donor, bispecific antibody comprising a PD-1 heavy chain variable region having an amino acid sequence as set forth in SEQ ID NO: 18 and a TGF-βRII heavy chain variable region having an amino acid sequence as set forth in SEQ ID NO: 23 did not inhibit TGF-βRII signaling in unstimulated CD4⁺ and CD8⁺ T cells and bispecific antibody comprising a PD-1 heavy chain variable region having an amino acid sequence as set forth in SEQ ID NO: 13 and a TGF-βRII heavy chain variable region having an amino acid sequence as set forth in SEQ ID NO: 47 inhibited TGF-βRII signaling in unstimulated CD4⁺ and CD8⁺ T cells significantly less potent than in stimulated CD4⁺ and CD8⁺ T cells. These data confirm the findings in Example 4 that the bispecific antibodies inhibit TGF-β-induced SMAD phosphorylation in a manner correlated to PD-1 expression.

### Example 6—Exhausted Mixed Lymphocyte Reaction (MLR) Assay

Bispecific antibodies were characterized in an Exhausted Mixed Lymphocyte Reaction (MLR) Assay to determine their potency in inducing cytokine production by exhausted T cells.

Antibody samples included several bispecific antibodies, negative control IgG1 antibody (RSV), reference PD-1 antibody pembrolizumab, an analog of reference TGF-βRII antibody TGF1, and a combination of reference PD-1 antibody pembrolizumab and an analog of reference TGF-βRII antibody TGF1.

Human PBMCs were isolated from healthy donors. In vitro T cell exhaustion was carried out by repeated activation of PBMCs by Staphylococcal enterotoxin B (SEB) for 6 days. Total T cells were isolated using T cell isolation kit (StemCell Technologies, cat #17951) according to manufacturer's instructions. T cells were mixed with dendritic cells (DC) from different donors at 10:1 T cell DC ratio in 96-well U bottom plates. Serial dilutions of antibody samples were added and the plates were incubated for six more days at 37° C./5% CO₂. After six days, IFN-γ, IL-2, or TNF-α cytokine level in the supernatant was measured using custom MSD kit. GraphPad Prism (8.2.0) was used to plot the graphs.

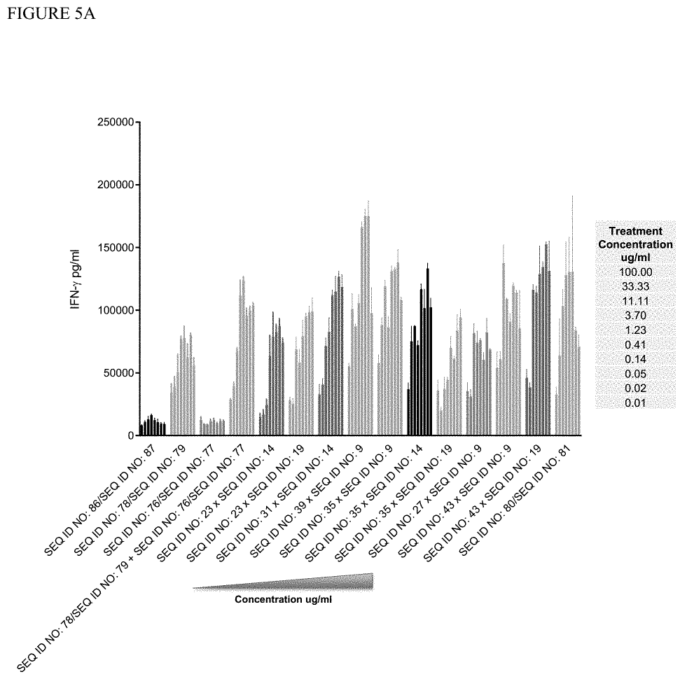

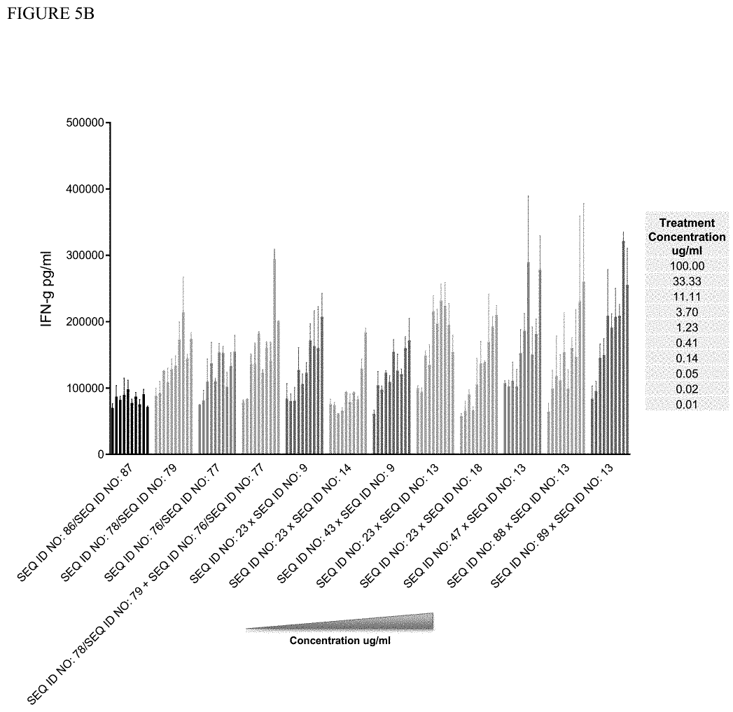

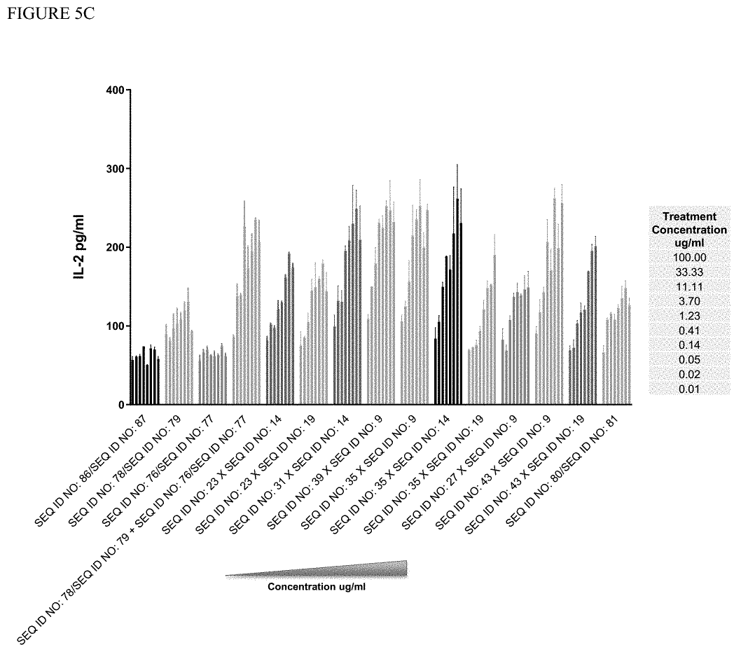

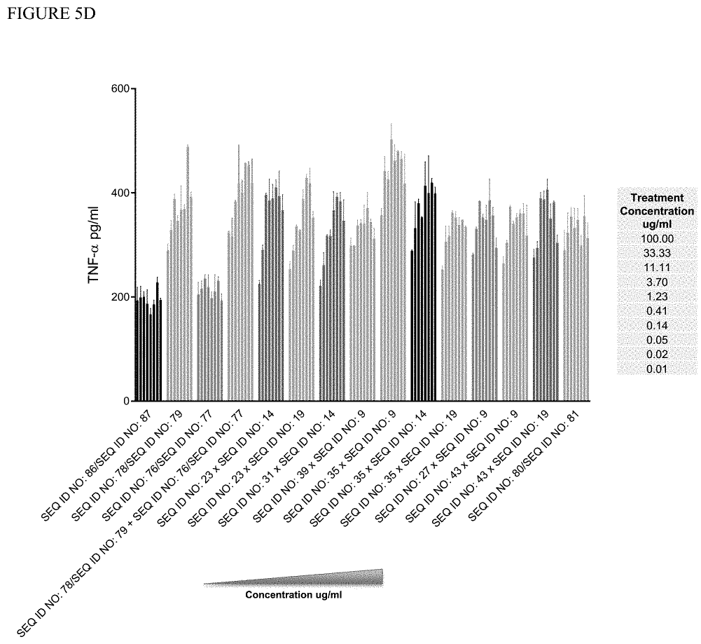

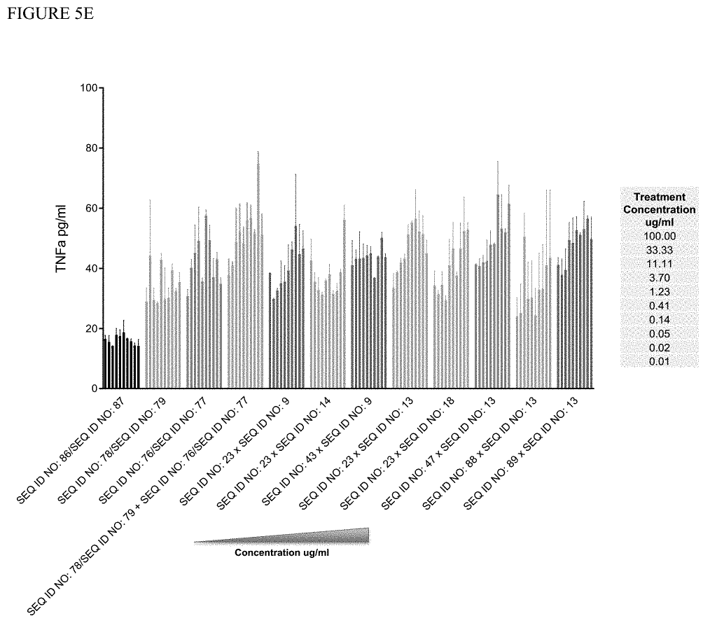

Representative results from two experiments with five donors each are shown in FIGS. 5A, C, and D. Results from a further experiment with three donors are shown in FIGS. 5B and E. Most of the bispecific antibodies induced similar levels of IFN-γ, IL-2, or TNF-α as reference PD-1 antibody pembrolizumab, an analog of reference PD-L1-TGF-β TRAP molecule, or a combination of reference PD-1 antibody pembrolizumab and reference TGF-βRII antibody TGF1. IC₅₀ values of a number of bispecific antibodies from the initial study are shown in Table 7.

TABLE 7
IC₅₀ values of the Exhausted MLR Assay

| | | AUC IFNg | AUC IL2 | AUC TNFa |
|---|---|---|---|---|
| | | (% of | (% of | (% of |
| | | Combi- | Combi- | Combi- |
| TGF-βRII | PD-1 | nation) | nation) | nation) |
| SEQ ID NO: 23 | SEQID NO: 14 | 95.3 | 97.1 | 92 |
| SEQ ID NO: 23 | SEQID NO: 19 | 121.3 | 98.5 | 93.2 |
| SEQ ID NO: 31 | SEQID NO: 14 | 137.2 | 105.4 | 100.4 |
| SEQ ID NO: 39 | SEQID NO: 9 | 139.3 | 105.9 | 101 |
| SEQ ID NO: 35 | SEQID NO: 9 | 132.5 | 101.9 | 96 |
| SEQ ID NO: 35 | SEQID NO: 14 | 121.2 | 104.3 | 94.5 |
| SEQ ID NO: 35 | SEQID NO: 19 | 94.1 | 91.8 | 78 |
| SEQ ID NO: 27 | SEQID NO: 9 | 90.1 | 86.7 | 76.4 |
| SEQ ID NO: 43 | SEQID NO: 9 | 98.5 | 104.3 | 83.1 |
| SEQ ID NO: 43 | SEQID NO: 19 | 95.7 | 89 | 77.5 |

### Example 7—HEK-BLUE-PD-1 TGF-β Reporter Assay

Bispecific antibodies were characterized in an HEK-BLUE-PD-1 TGF-β Reporter Assay to determine their potency in inhibiting TGF-β-induced signaling in T cells. Stimulation of HEK-Blue™ TGF-β cells or HEK-Blue™ TGF-β-PD-1⁺ cells with TGF-β induces the activation of the TGF-β/Smad signaling pathway, leading to the formation of a Smad3/Smad4 complex inducing the production of SEAP.

Reagents used: Recombinant Human TGF-beta 1 (Human Cell-expressed) Protein, R&D, Cat #7754-BH. Growth Medium: DMEM, 4.5 g/l glucose, 2 mM L-glutamine, 10% heat-inactivated fetal bovine serum (FBS; 30 min at 56° C.), 100 μg/ml Normocin™, Pen-Strep (100 U/ml-100 μg/ml). Growth Medium with puromycin 0.4 ug/mL for HEK-Blue™ TGF-β-PD-1⁺ cells. Test Medium: DMEM 4.5 g/l glucose, 2 mM L-glutamine, 0.1% heat-inactivated FBS, Pen-Strep (100 U/ml-100 μg/ml) without Normocin™, Blasticidin, Hygromycin B, and Zeocin™.

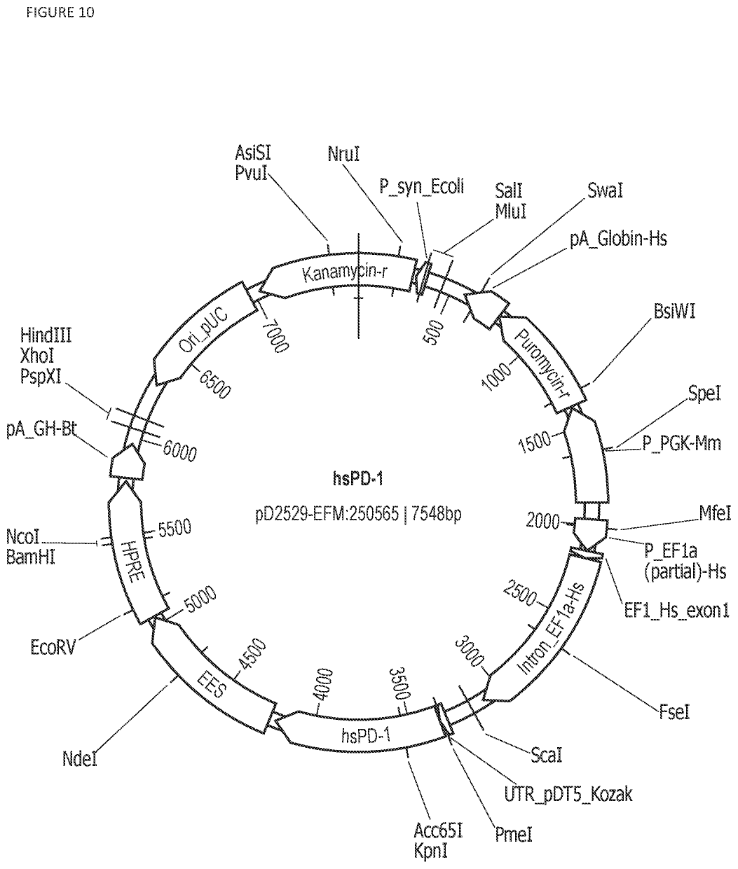

A stable PD-1-expressing HEK-Blue™ TGF-β cell line was generated as follows: the full length cds for PD-1 was inserted into the mammalian expression vector pD2529-EFM (ATUM) which contains the gene for puromycin resistance. The promoter is a modified EF1a. The vector construction was done by ATUM and the sequence was confirmed. See FIG. 10 for the vector map. HEK-Blue™ TGF-β cells (Invivogen), in which PD-1 expression was undetectable, were transfected using TransIT-293 transfection reagent (Mirus Bio) following manufacturer's protocols. Stably transfected cells were selected in HEK-Blue™ TGF-β media containing 0.4 ug/ml puromycin. Clones were isolated by limiting dilution and were characterized for PD-1 expression by western blot. One clone was selected based on 1) its stable and homogeneous surface PD-1 expression (one peak in the histogram plot); 2) having similar surface TGF-βRII expression as compared to the parental HEK-Blue™ cell line; and 3) having a similar EC50 as reference antibody TGF1 in a reporter assay. The GMFI of PD-1 in the selected clone was 3272 compared to 5 for the parental cell line and 8 for the isotype control. The number of PD-1 molecules on these cells was determined at about 20000, using quantibrite bead methodology.

Antibody samples included several bispecific antibodies, negative control IgG1 antibody (RSV), reference PD-1 antibody pembrolizumab, and an analog of reference TGF-βRII antibody TGF1.

HEK-Blue™ TGF-β cells (Invivogen, Catalog code: hkb-tgfb), expressing TGF-βRII, or HEK-Blue™ TGF-β-PD-1⁺ cells were seeded in a 96-well flat-bottom plate, 25000 cells per well in test medium. Serial dilutions of antibody samples were added and the cells were incubated for one hour at room temperature; followed by the addition of human recombinant TGF-β1 at a final concentration of 1 ng/ml. Cells were incubated at 37° C./5% CO₂ overnight. After incubation, 40 ul of supernatants were transferred from each well into a fresh flat-bottom 96-well plate; and 160 ul of re-suspended QUANTI-Blue™ Solution added to the supernatant. The plate was incubated at 37° C./5% CO₂ for 40 minutes. The quantity of SEAP secreted in the supernatant was assessed using QUANTI-Blue™ Solution, a SEAP detection reagent. SEAP levels were determined using a spectrophotometer at 650 nm. GraphPad Prism (8.2.0) was used to plot the graphs.

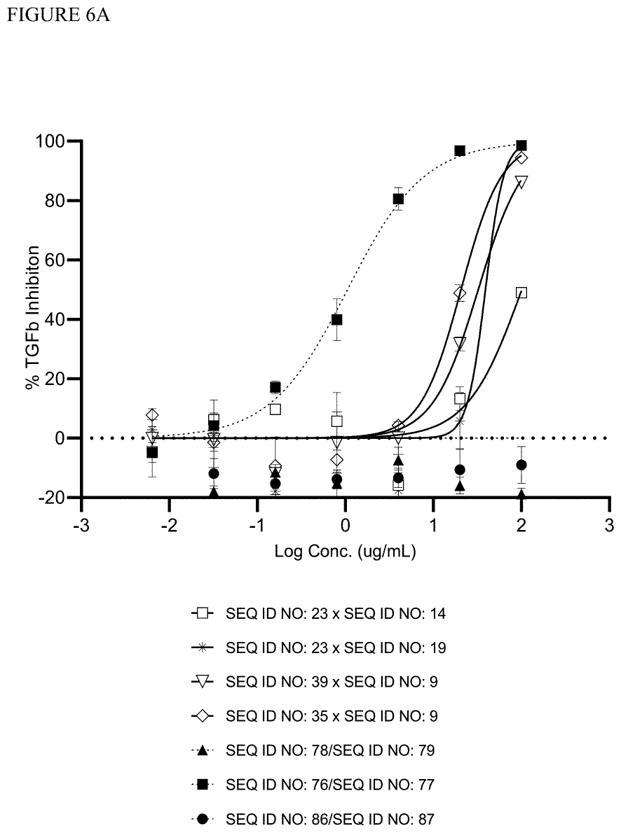

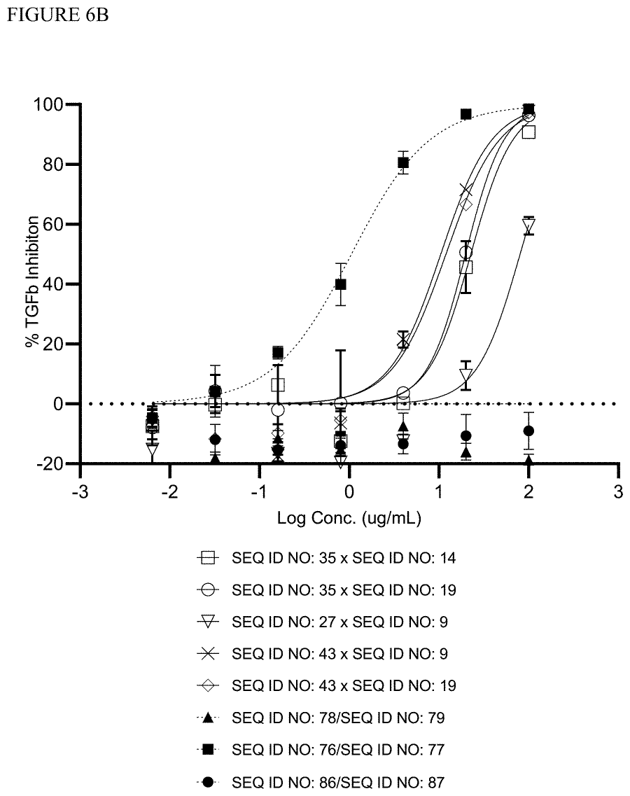

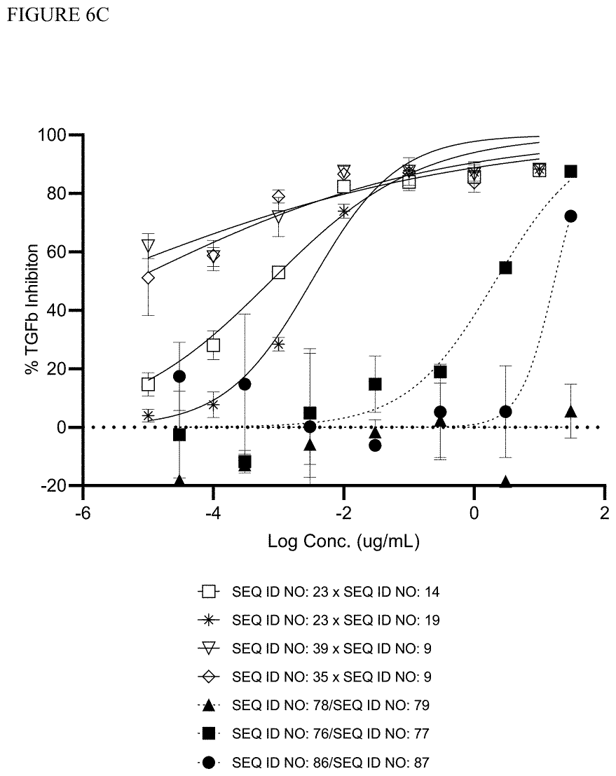

Results are shown in FIGS. 6A-6D and Table 8. A number of bispecific antibodies demonstrate potent inhibition of TGF-β-induced signaling in a manner correlated to PD-1 expression.

TABLE 8
Results from the HEK-BLUE-PD-1 TGF-β Reporter Assay.

| | HEK-Blue ™ | HEK-Blue ™ | Fold |
|---|---|---|---|
| Antibody | TGF-β | TGF-β -PD-1+ | difference |
| SEQ ID NO: 86/ | >100 | >100 | — |
| SEQ ID NO: 87 | | | |
| SEQ ID NO: 78/ | >100 | >100 | — |
| SEQ ID NO: 79 | | | |
| SEQ ID NO: 76/ | 0.8056 | 0.6551 | — |
| SEQ ID NO: 77 | | | |
| SEQ ID NO: 23 × | >100 (142) | 0.005606 | 25330 |
| SEQ ID NO: 14 | | | |
| SEQ ID NO: 23 × | >100 (177) | 0.001747 | 101317 |
| SEQ ID NO: 19 | | | |
| SEQ ID NO: 31 × | 9.7 | 7.404 | 1.3101 |
| SEQ ID NO: 14 | | | |
| SEQ ID NO: 39 × | 38 | 0.000193 | 197198 |
| SEQ ID NO: 9 | | | |
| SEQ ID NO: 35 × | 18.5 | 0.001533 | 12068 |
| SEQ ID NO: 9 | | | |
| SEQ ID NO: 35 × | 20.45 | 0.000687 | 20450 |
| SEQ ID NO: 14 | | | |
| SEQ ID NO: 35 × | 23.79 | 0.001215 | 19580 |
| SEQ ID NO: 19 | | | |
| SEQ ID NO: 27 × | 65 | 0.00141 | 46099 |
| SEQ ID NO: 9 | | | |
| SEQ ID NO: 43 × | 8.2 | <0.0001 | NA |
| SEQ ID NO: 9 | | | |
| SEQ ID NO: 43 × | 4.1 | <0.0001 | NA |
| SEQ ID NO: 19 | | | |

### Example 8—Treg Suppression Assay

Bispecific antibodies were characterized in a Treg Suppression Assay to determine their capability to eliminate or reduce the suppressive effect of regulatory T cells and thereby to induce cytokine production by T cells.

Antibody samples included several bispecific antibodies, negative control IgG1 antibody (RSV), reference PD-1 antibody pembrolizumab, an analog of reference TGF-βRII antibody TGF1, and a combination of reference PD-1 antibody pembrolizumab and an analog of reference TGF-βRII antibody TGF1.

Human PBMCs were isolated from healthy donors by Ficoll-Paque gradient centrifugation. Tregs were isolated using EasySep treg isolation kit (StemCell Technologies, #18063) according to manufacturer's instructions. Tregs were mixed with PBMCs from the same donor. Anti-CD3 ab (BD Biosciences #555336) and anti-CD28 an (BD Biosciences, #555725) were added to the coculture. Finally, serial dilutions of bispecific antibodies were added and the plates were incubated for three days at 37° C./5% CO₂. After three days of incubation, IFN-γ and TNF-α cytokine levels in the supernatant were measured using MSD kit (Mesoscale). GraphPad Prism (8.2.0) was used to plot the graphs.

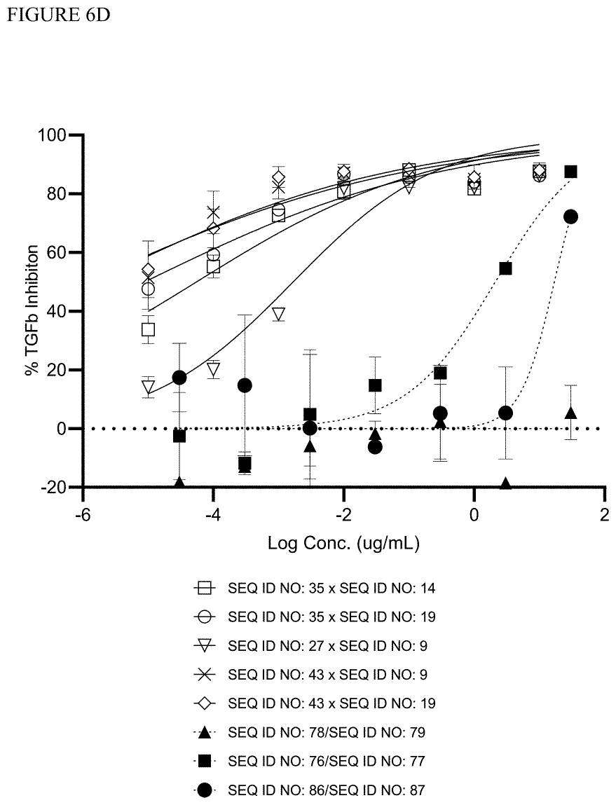

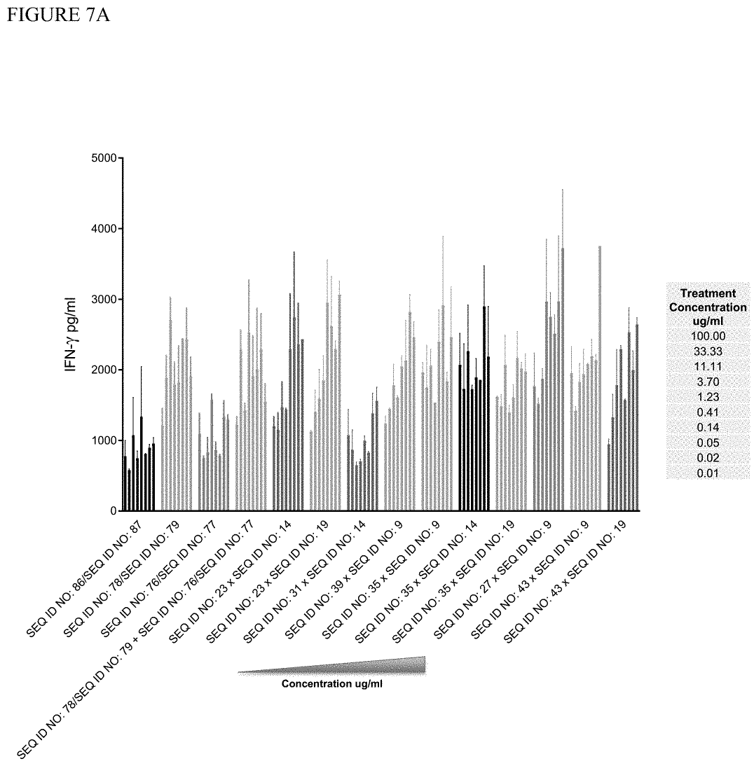

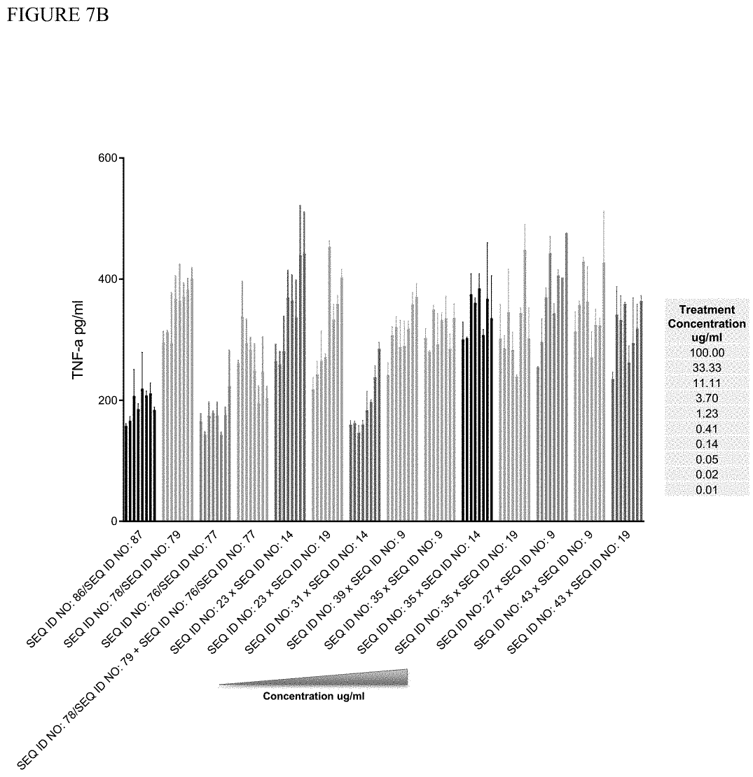

Results are shown in FIGS. 7A and 7B and Table 9. Most of the bispecific antibodies induced similar levels of IFN-γ or TNF-α as reference PD-1 antibody pembrolizumab, or a combination of reference PD-1 antibody pembrolizumab and the analog of reference TGF-βRII antibody TGF1.

TABLE 9
Results of the Treg Suppression Assay.

| | | AUC IFN-γ | AUC TNF-α |
|---|---|---|---|
| | | % of | % of |
| | | combination | combination |
| | | of reference | of reference |
| TGF-βRII | PD-1 | antibodies | antibodies |
| SEQ ID NO: 23 | SEQ ID NO: 14 | 95.1 | 134.5 |
| SEQ ID NO: 23 | SEQ ID NO: 19 | 122.5 | 179.4 |
| SEQ ID NO: 31 | SEQ ID NO: 14 | 81.8 | 101 |
| SEQ ID NO: 39 | SEQ ID NO: 9 | 107.2 | 138.9 |
| SEQ ID NO: 35 | SEQ ID NO: 9 | 99.9 | 121.5 |
| SEQ ID NO: 35 | SEQ ID NO: 14 | 123.9 | 121.7 |
| SEQ ID NO: 35 | SEQ ID NO: 19 | 88.3 | 115.8 |
| SEQ ID NO: 27 | SEQ ID NO: 9 | 103.8 | 127.2 |
| SEQ ID NO: 43 | SEQ ID NO: 9 | 109 | 114 |
| SEQ ID NO: 43 | SEQ ID NO: 19 | 128.6 | 107.2 |

### Example 9—Macrophage Suppression Assay

Tumor-associated macrophages of the M2 phenotype inhibit T cell proliferation and cytokine production. Bispecific antibodies were characterized in an M2 macrophage suppression assay to test if they can reverse the inhibitory effect of M2 macrophages on T cell proliferation and IFN-γ production.

Bispecific antibodies were tested along with negative control IgG1 antibody (RSV), an analog of reference TGF-βRII antibody TGF1, an analog of reference PD-L1-TGF-β TRAP molecule, an analog of reference PD-1 antibody nivolumab, commercial reference PD-1 antibody Opdivo (BMS), positive control antibody anti-LILRB2 (Biolegend), rat IgG2a isotype control (Biolegend) and huIgG4 isotype control (Biolegend). In addition, a co-culture of stimulated CD4⁺ T cells and M2 macrophages without the addition of test or control antibodies or CD4⁺ T cells alone (unstimulated versus stimulated), were included as control conditions in the assay.

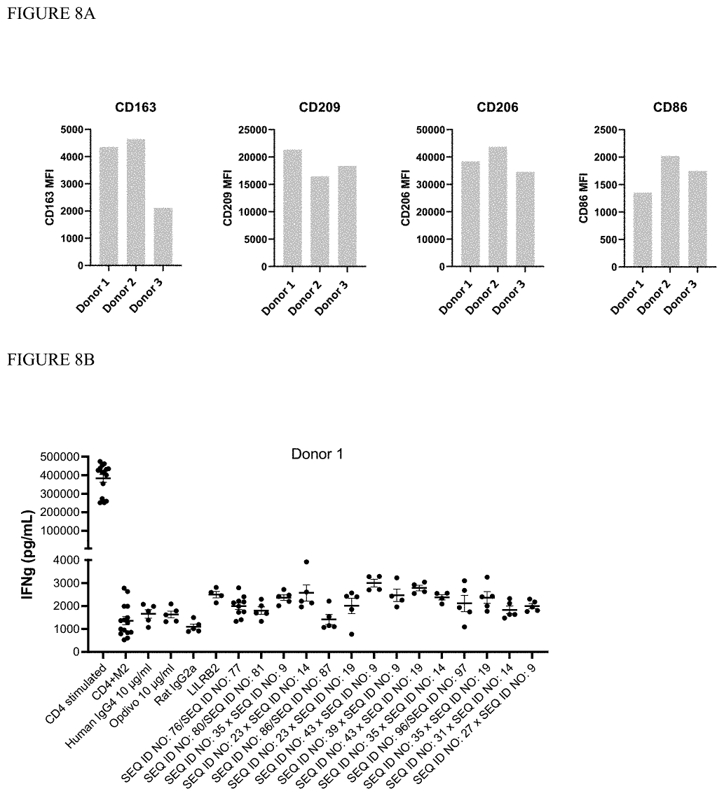

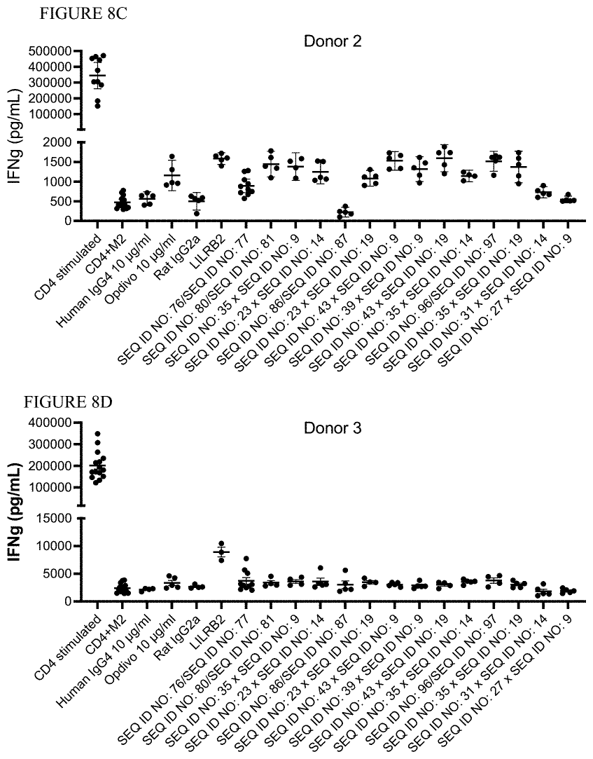

PBMC-isolated monocytes from three different healthy donors were differentiated into macrophages with M-CSF for six days and polarized using a specific cocktail of the cytokines IL-4 (20 ng/ml), IL-10 (20 ng/ml) and TGF-β (20 ng/ml) (+M-CSF) to obtain M2 macrophages. To confirm the phenotype, the expression of CD163 (Miltenye Biotec), CD209 (Miltenye Biotec), CD206 (BD Bioscience) and CD86 (Miltenye Biotec) was measured by flow cytometry (FIGS. 8A-8D). CD163 is expressed by Ms-like macrophages in all donors. CD209 and CD206 are highly expressed by M2-macrophages in all donors. CD86 is expressed at a low level by M2-like macrophages. M2-like macrophages show the expected phenotype in all three donors.

Next, the M2 macrophages were activated with LPS (100 ng/ml) for 4 hours. The macrophages were harvested, washed, and seeded in 5-plicates in 96-well plates with autologous CD4⁺ activated T cells (activated by CD3/CD28 ImmunoCult™ from StemCell technologies) in a 1:5 ratio in the presence of the test or control antibodies at 10 μg/ml concentration. On day 5 of the co-culture, the concentration of secreted IFN-γ was measured by ELISA (LEGEND MAX™ Human IFN-γ ELISA Kit, Biolegend). Data was analyzed in GraphPad Prism 7.0 using multi-way ANOVA.

Results are shown in FIGS. 8A-8D. A number of bispecific antibodies induced similar or greater levels of IFN-γ as compared to the analog of reference PD-1 antibody nivolumab, the analog of reference TGF-βRII antibody TGF1, or the analog of reference PD-L1-TGF-β TRAP molecule.

### Example 10—In Vivo Humanized NSG MDA-MB-231 Mouse Model

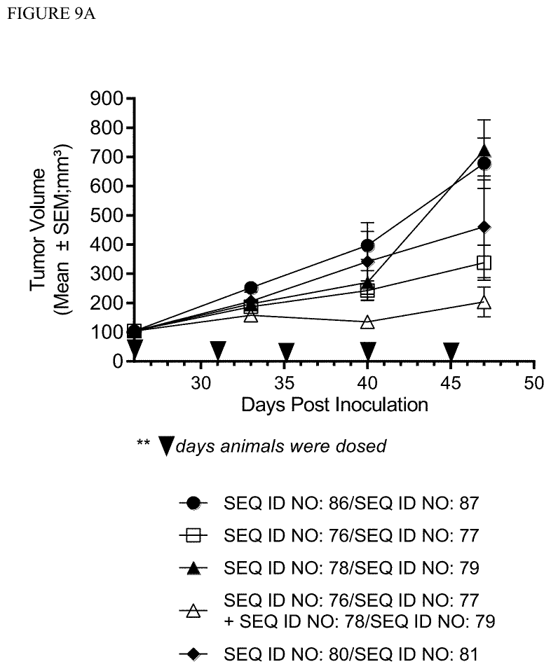

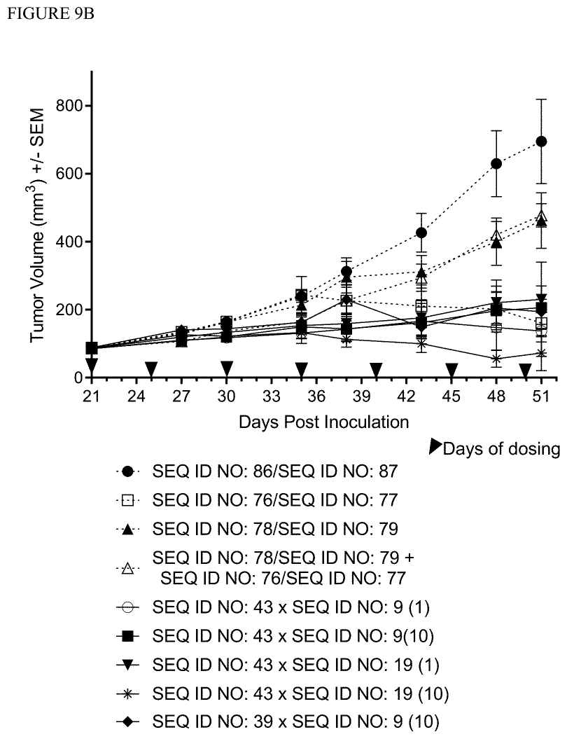

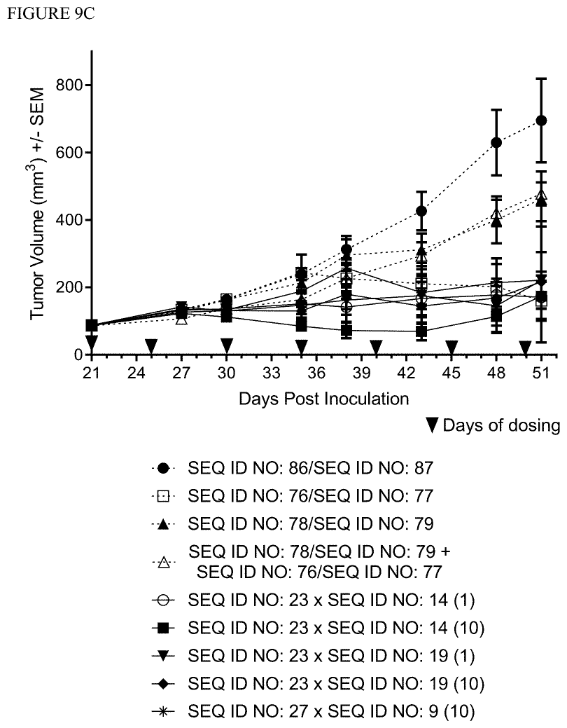

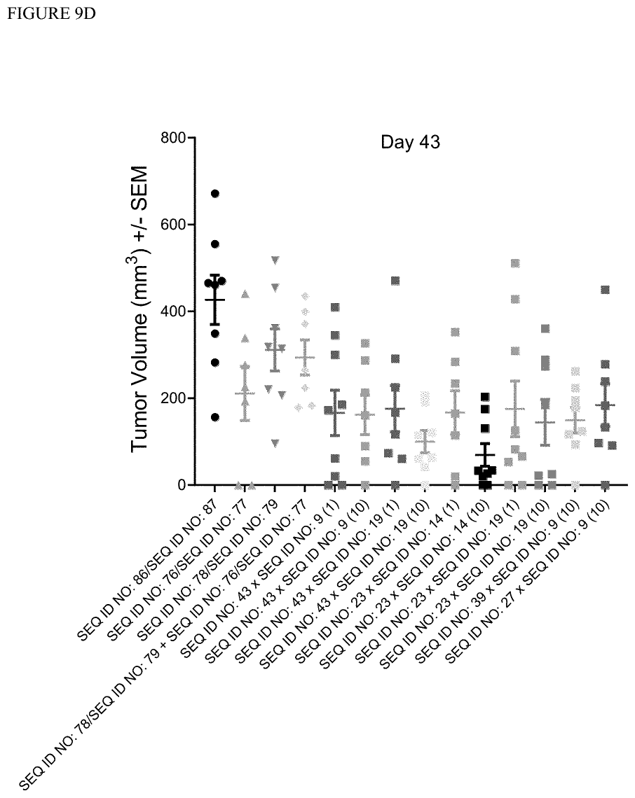

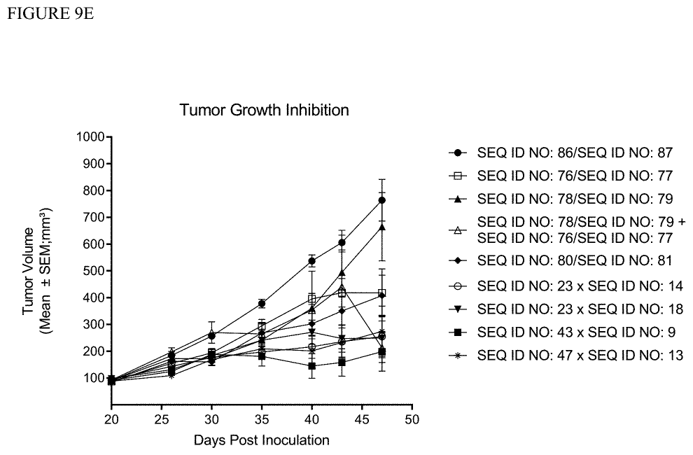

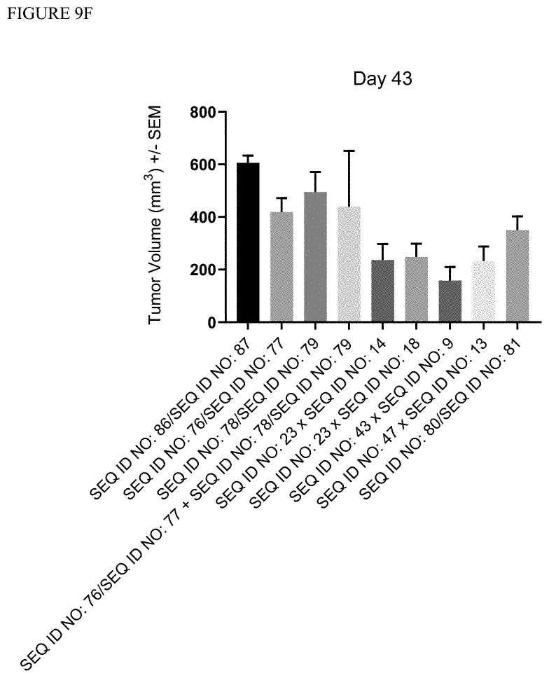

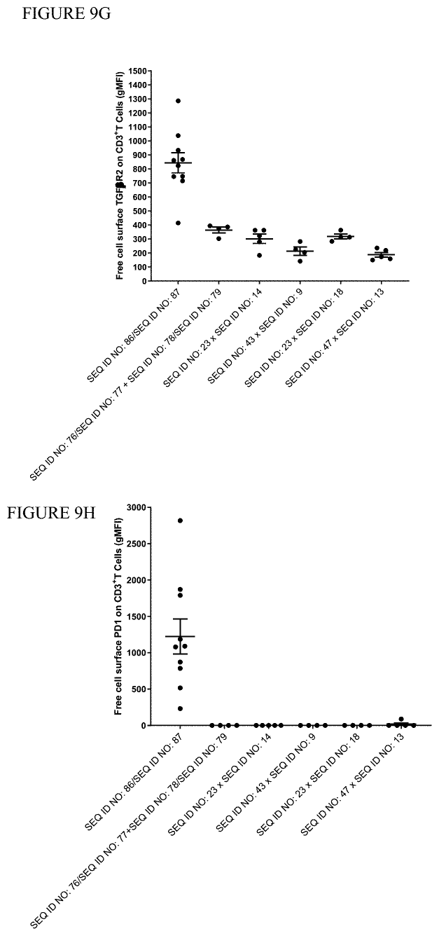

Bispecific antibodies were characterized in vivo in a humanized NSG MDA-MB-231 mouse model to determine their potency in reducing tumor volume. This mouse model was validated using 10 mg/kg of a negative control bivalent monospecific IgG1 antibody comprising a heavy chain having an amino acid sequence as set forth in SEQ ID NO: 86 and a light chain having an amino acid sequence as set forth in SEQ ID NO: 87; an analog of reference TGF-βRII antibody TGF1 (10 mg/kg); reference PD-1 antibody pembrolizumab (10 mg/kg); a combination of analog of reference TGF-βRII antibody TGF1 (10 mg/kg) and reference PD-1 antibody pembrolizumab (10 mg/kg); and an analog of reference PD-L1-TGF-β TRAP molecule (10 mg/kg). Results are shown in FIG. 9A.

Humanized CD34 NSG mice were inoculated subcutaneously with a total of 3×10⁶ MDA-MB-231 tumor cells suspended in 100 μl of serum-free culture medium and matrigel matrix (Corning) in equal volumes. After tumors were established (80-100 mm³), the mice were randomized into the following treatment groups:

1) Negative control bivalent monospecific IgG1 antibody comprising a heavy chain having an amino acid sequence as set forth in SEQ ID NO: 86 and a light chain having an amino acid sequence as set forth in SEQ ID NO: 87 (10 mg/kg);

2) An analog of reference TGF-βRII antibody TGF1 (10 mg/kg);

3) Reference PD-1 antibody pembrolizumab (10 mg/kg);

4) An analog of reference TGF-βRII antibody TGF1 (10 mg/kg)+reference PD-1 antibody pembrolizumab (10 mg/kg);

5) Bispecific antibody comprising a TGF-βRII binding domain with a heavy chain variable region having an amino acid sequence as set forth in SEQ ID NO: 43 and a PD-1 binding domain with a heavy chain variable region having an amino acid sequence as set forth in SEQ ID NO: 9 (1 mg/kg);

6) Bispecific antibody comprising a TGF-βRII binding domain with a heavy chain variable region having an amino acid sequence as set forth in SEQ ID NO: 43 and a PD-1 binding domain with a heavy chain variable region having an amino acid sequence as set forth in SEQ ID NO: 9 (10 mg/kg);

7) Bispecific antibody comprising a TGF-βRII binding domain with a heavy chain variable region having an amino acid sequence as set forth in SEQ ID NO:43 and a PD-1 binding domain with a heavy chain variable region having an amino acid sequence as set forth in SEQ ID NO: 19 (1 mg/kg);

8) Bispecific antibody comprising a TGF-βRII binding domain with a heavy chain variable region having an amino acid sequence as set forth in SEQ ID NO: 43 and a PD-1 binding domain with a heavy chain variable region having an amino acid sequence as set forth in SEQ ID NO: 19 (10 mg/kg);

9) Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 14 (1 mg/kg);

10) Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 14 (10 mg/kg);

11) Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 19 (1 mg/kg);

12) Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 19 (10 mg/kg);

13) Bispecific antibody comprising a TGF-βRII binding domain with a heavy chain variable region having an amino acid sequence as set forth in SEQ ID NO: 39 and a PD-1 binding domain with a heavy chain variable region having an amino acid sequence as set forth in SEQ ID NO: 9 (10 mg/kg);

14) Bispecific antibody comprising a TGF-βRII binding domain with a heavy chain variable region having an amino acid sequence as set forth in SEQ ID NO: 27 and a PD-1 binding domain with a heavy chain variable region having an amino acid sequence as set forth in SEQ ID NO: 9 (10 mg/kg);

15) Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 18 (10 mg/kg);

16) Bispecific antibody comprising a TGF-βRII binding domain with a heavy chain variable region having an amino acid sequence as set forth in SEQ ID NO: 47 and a PD-1 binding domain with a heavy chain variable region having an amino acid sequence as set forth in SEQ ID NO: 13 (10 mg/kg).

Each group had 8-9 mice. Animals were dosed intraperitoneally every five days for a period of 27 or 30 days. Tumors were measured using calipers, and the tumor volume was calculated by assimilating them to an ellipsoid using the formula: 1(length)×w² (width)×½. Body weights were also monitored all through the study. Tumors were harvested (24 hours post last dosing) for tumor immune profiling and receptor occupancy post termination of the study.

Results are shown in FIG. 9B-E. All bispecific antibodies induced a superior anti-tumor response than a combination of reference PD-1 antibody pembrolizumab and the analog of reference TGF-βRII antibody TGF1. Bispecific antibodies even induced a superior anti-tumor response at both 1 mg/kg and 10 mg/kg dosage levels whereas the combination of reference antibodies included a dosage of 10 mg/kg of each reference antibody.

Along with efficacy, similar receptor coverage of both PD-1 and TGF-βRII receptors upon bispecific antibody treatment was observed as with the combination treatment of the analog of reference TGF-βRII antibody TGF1 and reference PD-1 antibody pembrolizumab on T cells analyzed 24 hours post last dose (FIG. 9F).

### Example 11—Testing Different Doses in an In Vivo Humanized NSG MDA-MB-231 Mouse Model

Bispecific antibody comprising a TGF-βRII binding domain with a heavy chain variable region having an amino acid sequence as set forth in SEQ ID NO: 23 and a PD-1 binding domain with a heavy chain variable region having an amino acid sequence as set forth in SEQ ID NO: 18 was characterized at two different dose levels, 1 mg/kg and 10 mg/kg, in vivo in a humanized NSG MDA-MB-231 mouse model, as described in Example 10. Negative control bivalent monospecific IgG1 antibody comprising a heavy chain having an amino acid sequence as set forth in SEQ ID NO: 86 and a light chain having an amino acid sequence as set forth in SEQ ID NO: 87 was included at 10 mg/kg.

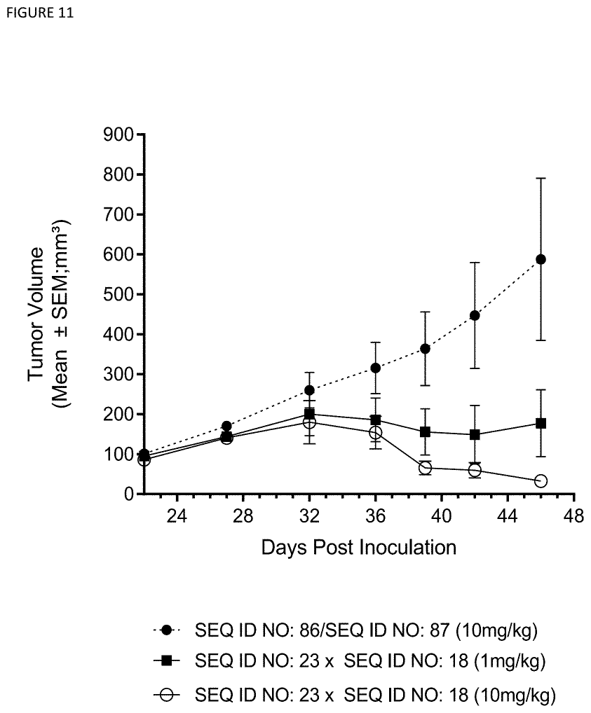

Results are shown in FIG. 11. The bispecific antibody induced a significant anti-tumor response at both dose levels.

Sequences

Heavy chain variable region
SEQ ID NO: 1
EVQLVQSGAEVKKPGSSMKVSCKASGGTFSSYVISWVRQAPGQGLEWMGMIIPVFDTSSYEKKFQGRITIIADKS
TSTVYLELSSLRSEDAAVYYCARGTVEATLLFDFWGQGTLVTVSS
Heavy chain CDR1
SEQ ID NO: 2
SYVIS
Heavy chain CDR2
SEQ ID NO: 3
MIIPVFDTSSYEKKFQG
Heavy chain CDR3
SEQ ID NO: 4
GTVEATLLFDF
Heavy chain variable region
SEQ ID NO: 5
QVQLQESGPGLVKPSETLSLTCTVSNGSLGFDFWSWIRQPPGRGLEWIGYIYYSGSWSLNPSFKGRVTMSVDTSK
NQFSLNLRSVTAADTAVYYCARGGYTGYGGDWFDPWGQGTLVTVSS
Heavy chain CDR1
SEQ ID NO: 6
FDFWS
Heavy chain CDR2
SEQ ID NO: 7
YIYYSGSWSLNPSFKG
Heavy chain CDR3
SEQ ID NO: 8
GGYTGYGGDWFDP
Heavy chain variable region
SEQ ID NO: 9
QVQLQESGPGLVKPSETLSLTCTVSDGSIGYHFWSWIRQPPGRGLEWIGYIVYSGSYNVNPSLKTRVTMSVDTSK
NQFSLNLRSVTAADTAVYYCARGGYTGYGGDWFDPWGQGTLVTVSS
Heavy chain CDR1
SEQ ID NO: 10
YHFWS
Heavy chain CDR2
SEQ ID NO: 11
YIVYSGSYNVNPSLKT
Heavy chain CDR3
SEQ ID NO: 12
GGYTGYGGDWFDP
Heavy chain variable region
SEQ ID NO: 13
QVQLQESGPGLVKPSETLSLTCTVSEGSIGYHFWSWIRQPPGRGLEWIGYIVYSGSYNVNPSLKTRVTMSVDTSK
NQFSLNLRSVTAADTAVYYCARGGYTGYGGDWFDPWGQGTLVTVSS
Heavy chain variable region
SEQ ID NO: 14
QVQLVQSGSELKKPGASVKVSCKASGYTFTRFALHWVRQAPGQGLEWMGWIDPNTGTPTFAQGVTGRFVFSLDTS
VTTAYLQISSLKAEDTAVYYCARSLGYCDSDICYPNWIFDNWGQGTLVTVSS
Heavy chain CDR1
SEQ ID NO: 15
RFALH
Heavy chain CDR2
SEQ ID NO: 16
WIDPNTGTPTFAQGVTG
Heavy chain CDR3
SEQ ID NO: 17
SLGYCDSDICYPNWIFDN
Heavy chain variable region
SEQ ID NO: 18
QVQLVQSGSELKKPGASVKVSCKASGYTFTRFALHWVRQAPGQGLEWMGWIDPNTGTPTFAQGVTGRFVFSLDTS
VTTAYLQISSLKAEDTAVYYCARSLGYCDSDICYPNWIFDNWGQGTLVTVSS
Heavy chain variable region
SEQ ID NO: 19
QVQLVQSGSELKKPGASVKVSCKASGYTFTRFALSWVRQAPGQGLEWMGWIDPNTGTPTYAQDFTGRFVFSLDTS
VTTAYLQISSLKAEDTAVYYCARSLGYCGSDICYPNGILDNWGQGTLVTVSS
Heavy chain CDR1
SEQ ID NO: 20
RFALS
Heavy chain CDR2
SEQ ID NO: 21
WIDPNTGTPTYAQDFTG
Heavy chain CDR3
SEQ ID NO: 22
SLGYCGSDICYPNGILDN
Heavy chain variable region
SEQ ID NO: 23
EVQLVESGGGLVQPGGSLRLSCAASGFTFDIYAMTWVRQAPGKGLEWVSVISGSGGTTYYADSVKGRFTISRDNS
KNTLYLQMNSLRAEDTAVYYCARRGQYRDIVGATDYWGQGTLVTVSS
Heavy chain CDR1
SEQ ID NO: 24
IYAMT
Heavy chain CDR2
SEQ ID NO: 25
VISGSGGTTYYADSVKG
Heavy chain CDR3
SEQ ID NO: 26
RGQYRDIVGATDY
Heavy chain variable region
SEQ ID NO: 27
EVQLVESGGGLVQPGGSLRLSCAASGFTFDINAMTWVRQAPGKGLEWVSVISGSGGTTYYADSVKGRFTISRDNS
KNTLYLQMNSLRAEDTAVYYCARRGQYRDIVGATDYWGQGTLVTVSS
Heavy chain CDR1
SEQ ID NO: 28
INAMT
Heavy chain CDR2
SEQ ID NO: 29
VISGSGGTTYYADSVKG
Heavy chain CDR3
SEQ ID NO: 30
RGQYRDIVGATDY
Heavy chain variable region
SEQ ID NO: 31
QVQLVESGGGLVEPGGSLRLSCAASGFTFSNAWMSWVRQAPGKGLEWVGRIKTTISGGATDFAAPVKGRFTISRD
DSKNTLYLQMNSLKTEDTAVYYCTLDLRDYWGQGTLVTVSS
Heavy chain CDR1
SEQ ID NO: 32
NAWMS
Heavy chain CDR2
SEQ ID NO: 33
RIKTTISGGATDFAAPVKG
Heavy chain CDR3
SEQ ID NO: 34
DLRDY
Heavy chain variable region
SEQ ID NO: 35
QVQLVESGGGLVEPGGSLRLSCAASGFKFSNAWMSWVRQAPGKGLEWVGRIKTTISGGATQFAAPVKGRFTISRD
DSKNTLYLQMNSLKTEDTAVYYCTLDLRDYWGQGTLVTVSS
Heavy chain CDR1
SEQ ID NO: 36
NAWMS
Heavy chain CDR2
SEQ ID NO: 37
RIKTTISGGATQFAAPVKG
Heavy chain CDR3
SEQ ID NO: 38
DLRDY
Heavy chain variable region
SEQ ID NO: 39
QVQLVESGGGLVQPGGSLRLSCAVSGFTFRRYAMSWVRQAPGKGLEWVSAISASGDRTHNTDSVKGRFSISRDNS
KNTLYLQMNSLRAEDTAVYFCAKGIAASGKNYFDPWGQGTLVTVSS
Heavy chain CDR1
SEQ ID NO: 40
RYAMS
Heavy chain CDR2
SEQ ID NO: 41
AISASGDRTHNTDSVKG
Heavy chain CDR3
SEQ ID NO: 42
GIAASGKNYFDP
Heavy chain variable region
SEQ ID NO: 43
QVQLVESGGGLVQPGGSLRLSCAVSGFTFSRYAMSWVRQAPGKGLEWVSAISASGDRTKNTDSVKGRFSISRDNS
KNTLYLQMNSLRAEDTAVYFCAKGTAAAGKNYFDPWGQGTLVTVSS
Heavy chain CDR1
SEQ ID NO: 44
RYAMS
Heavy chain CDR2
SEQ ID NO: 45
AISASGDRTKNTDSVKG
Heavy chain CDR3
SEQ ID NO: 46
GTAAAGKNYFDP
Heavy chain variable region
SEQ ID NO: 47
QVQLVESGGGLVQPGGSLRLSCAVSGFTFSRYAMSWVRQAPGKGLEWVSAISASGDRTKYTDSVKGRFSISRDNS
KNTLYLQMNSLRAEDTAVYFCAKGTAAAGKNYFDPWGQGTLVTVSS
Light chain variable region
SEQ ID NO: 48
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPTFGQGTKVEIK
Light chain CDR1 according to IMGT
SEQ ID NO: 49
QSISSY
Light chain CDR2 according to IMGT
SEQ ID NO: 50
AAS
Light chain CDR3 according to IMGT
SEQ ID NO: 51
QQSYSTPPT
Light chain variable region
SEQ ID NO: 52
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
Light chain CDR1 according to IMGT
SEQ ID NO: 53
QSISSY
Light chain CDR2 according to IMGT
SEQ ID NO: 54
AAS
Light chain CDR3 according to IMGT
SEQ ID NO: 55
QQSYSTPPIT
Light chain variable region
SEQ ID NO: 56
EIVMTQSPATLSVSPGERATLSCRASQSVSSNLAWYQQKPGQAPRLLIYGASTRATGIPARFSGSGSGTEFTLTI
SSLQSEDFAVYYCQQYNNWPWTFGQGTKVEIK
Light chain CDR1 according to IMGT
SEQ ID NO: 57
QSVSSN
Light chain CDR2 according to IMGT
SEQ ID NO: 58
GAS
Light chain CDR3 according to IMGT
SEQ ID NO: 59
QQYNNWPWT
Light chain variable region
SEQ ID NO: 60
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIK
Light chain CDR1 according to IMGT
SEQ ID NO: 61
QSVSSSY
Light chain CDR2 according to IMGT
SEQ ID NO: 62
GAS
Light chain CDR3 according to IMGT
SEQ ID NO: 63
QQYGSSPWT
Light chain variable region
SEQ ID NO: 64
SYVLTQPPSVSVAPGETARITCGGDNIGRKSVYWYQQKSGQAPVLVIYYDSDRPSGIPERFSGSNSGNTATLTIS
RVEAGDEADYYCQVWDGSSDHWVFGGGTKLTVL
Light chain CDR1 according to IMGT
SEQ ID NO: 65
NIGRKS
Light chain CDR2 according to IMGT
SEQ ID NO: 66
YDS
Light chain CDR3 according to IMGT
SEQ ID NO: 67
QVWDGSSDHWV
hinge region
SEQ ID NO: 68
EPKSCDKTHTCPPCP
CH1 region
SEQ ID NO: 69
ASTKGPSVFPLAPSSKSTSGGTAALGCLVKDYFPEPVTVSWNSGALTSGVHTFPAVLQSSGLYSLSSWTVPSSSL
GTQTYICNVNHKPSNTKVDKRV
CH2 region
SEQ ID NO: 70
APELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAK
CH2-DM region
SEQ ID NO: 71
APELGRGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAK
CH3 region
SEQ ID NO: 72
GQPREPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPG
CH3-DE region
SEQ ID NO: 73
GQPREPQVYTDPPSREEMTKNQVSLTCEVKGFYPSDIAVEWESNGQPENNYKTTPPVLDSDGSFFLYSKLTVDKS
RWQQGNVFSCSVMHEALHNHYTQKSLSLSPG
CH3-KK region
SEQ ID NO: 74
GQPREPQVYTKPPSREEMTKNQVSLKCLVKGFYPSDIAVEWESNGQPENNYKTTPPVLDSDGSFFLYSKLTVDKS
RWQQGNVFSCSVMHEALHNHYTQKSLSLSPG
CL region
SEQ ID NO: 75
RTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Heavy chain reference anti-TGF-βRII antibody TGF1 analog
SEQ ID NO: 76
QLQVQESGPGLVKPSETLSLTCTVSGGSISNSYFSWGWIRQPPGKGLEWIGSFYYGEKTYYNPSLKSRATISIDT
SKSQFSLKLSSVTAADTAVYYCPRGPTMIRGVIDSWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG
K
Light chain reference anti-TGF-βRII antibody TGF1 analog
SEQ ID NO: 77
EIVLTQSPATLSLSPGERATLSCRASQSVRSYLAWYQQKPGQAPRLLIYDASNRATGIPARFSGSGSGTDFTLTI
SSLEPEDFAVYYCQQRSNWPPTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
Heavy chain reference PD-1 antibody pembrolizumab
SEQ ID NO: 78
QVQLVQSGVEVKKPGASVKVSCKASGYTFTNYYMYWVRQAPGQGLEWMGGINPSNGGTNFNEKFKNRVTLTTDSS
TTTAYMELKSLQFDDTAVYYCARRDYRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPP
CPPCPAPEFLGGPSVFLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKSLSLSLGK
Light chain reference PD-1 antibody pembrolizumab
SEQ ID NO: 79
EIVLTQSPATLSLSPGERATLSCRASKGVSTSGYSYLHWYQQKPGQAPRLLIYLASYLESGVPARFSGSGSGTDF
TLTISSLEPEDFAVYYCQHSRDLPLTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
Heavy chain reference PD-L1-TGF-β TRAP molecule analog
SEQ ID NO: 80
EVQLLESGGGLVQPGGSLRLSCAASGFTFSSYIMMWVRQAPGKGLEWVSSIYPSGGITFYADTVKGRFTISRDNS
KNTLYLQMNSLRAEDTAVYYCARIKLGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGA
GGGGSGGGGSGGGGSGGGGSGIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSSDECNDNIIFSEE
YNTSNPD
Light chain reference PD-L1TGF-β TRAP molecule analog
SEQ ID NO: 81
QSALTQPASVSGSPGQSITISCTGTSSDVGGYNYVSWYQQHPGKAPKLMIYDVSNRPSGVSNRFSGSKSGNTASL
TISGLQAEDEADYYCSSYTSSSTRVFGTGTKVTVLGQPKANPTVTLFPPSSEELQANKATLVCLISDFYPGAVTV
AWKADGSPVKAGVETTKPSKQSNNKYAASSYLSLTPEQWKSHRSYSCQVTHEGSTVEKTVAPTECS
human TGF-βRII isoform A
SEQ ID NO: 82
MGRGLLRGLWPLHIVLWTRIASTIPPHVQKSVNNDMIVTDNNGAVKFPQLCKFCDVRFSTCDNQKSCMSNCSITS
ICEKPQEVCVAVWRKNDENITLETVCHDPKLPYHDFILEDAASPKCIMKEKKKPGETFFMCSCSSDECNDNIIFS
EEYNTSNPDLLLVIFQVTGISLLPPLGVAISVIIIFYCYRVNRQQKLSSTWETGKTRKLMEFSEHCAIILEDDRS
DISSTCANNINHNTELLPIELDTLVGKGRFAEVYKAKLKQNTSEQFETVAVKIFPYEEYASWKTEKDIFSDINLK
HENILQFLTAEERKTELGKQYWLITAFHAKGNLQEYLTRHVISWEDLRKLGSSLARGIAHLHSDHTPCGRPKMPI
VHRDLKSSNILVKNDLTCCLCDFGLSLRLDPTLSVDDLANSGQVGTARYMAPEVLESRMNLENVESFKQTDVYSM
ALVLWEMTSRCNAVGEVKDYEPPFGSKVREHPCVESMKDNVLRDRGRPEIPSFWLNHQGIQMVCETLTECWDHDP
EARLTAQCVAERFSELEHLDRLSGRSCSEEKIPEDGSLNTTK
extracellular domain of human TGFβRII isoform A
SEQ ID NO: 83
TIPPHVQKSVNNDMIVTDNNGAVKFPQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNIIFSEEYNTSNPDLLLVIFQ
human TGF-βRII isoform B
SEQ ID NO: 84
MGRGLLRGLWPLHIVLWTRIASTIPPHVQKSDVEMEAQKDEIICPSCNRTAHPLRHINNDMIVTDNNGAVKFPQL
CKFCDVRFSTCDNQKSCMSNCSITSICEKPQEVCVAVWRKNDENITLETVCHDPKLPYHDFILEDAASPKCIMKE
KKKPGETFFMCSCSSDECNDNIIFSEEYNTSNPDLLLVIFQVTGISLLPPLGVAISVIIIFYCYRVNRQQKLSST
WETGKTRKLMEFSEHCAIILEDDRSDISSTCANNINHNTELLPIELDTLVGKGRFAEVYKAKLKQNTSEQFETVA
VKIFPYEEYASWKTEKDIFSDINLKHENILQFLTAEERKTELGKQYWLITAFHAKGNLQEYLTRHVISWEDLRKL
GSSLARGIAHLHSDHTPCGRPKMPIVHRDLKSSNILVKNDLTCCLCDFGLSLRLDPTLSVDDLANSGQVGTARYM
APEVLESRMNLENVESFKQTDVYSMALVLWEMTSRCNAVGEVKDYEPPFGSKVREHPCVESMKDNVLRDRGRPEI
PSFWLNHQGIQMVCETLTECWDHDPEARLTAQCVAERFSELEHLDRLSGRSCSEEKIPEDGSLNTTK
extracellular domain of isoform B of human TGF-βRII
SEQ ID NO: 85
TIPPHVQKSDVEMEAQKDEIICPSCNRTAHPLRHINNDMIVTDNNGAVKFPQLCKFCDVRFSTCDNQKSCMSNCS
ITSICEKPQEVCVAVWRKNDENITLETVCHDPKLPYHDFILEDAASPKCIMKEKKKPGETFFMCSCSSDECNDNI
IFSEEYNTSNPDLLLVIFQ
Heavy chain negative control RSV IgG1 antibody
SEQ ID NO: 86
EVQLVESGGGVVQPGRSLRLSCAASGFTFSNYGMHWVRQAPGKGLEWVAVISYDGSTKYSADSLKGRFTISRDNS
KNTLYLQMNSLRADDTAVYYCAKEGWSFDSSGYRSWFDSWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKSLS
LSPG
Light chain
SEQ ID NO: 87
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
Heavy chain variable region
SEQ ID NO: 88
QVQLVESGGGLVQPGGSLRLSCAVSGFTFSRYAMSWVRQAPGKGLEWVSAISASGDRTKNTDSVKGRFSISRDNS
KNTLYLQMNSLRAEDTAVYYCAKGTAAAGKNYFDPWGQGTLVTVSS
Heavy chain variable region
SEQ ID NO: 89
QVQLVESGGGLVQPGGSLRLSCAVSGFTFSRYAMSWVRQAPGKGLEWVSAISASGDRTKYTDSVKGRFSISRDNS
KNTLYLQMNSLRAEDTAVYYCAKGTAAAGKNYFDPWGQGTLVTVSS
Heavy chain CDR1
SEQ ID NO: 90
RYAMS
Heavy chain CDR2
SEQ ID NO: 91
AISASGDRTKYTDSVKG
Heavy chain CDR3
SEQ ID NO: 92
GTAAAGKNYFDP
V region VK1-39
SEQ ID NO: 93
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTP
VK1-39/JK1
SEQ ID NO: 94
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPTFGQGTKVEIK
VK1-39/JK5
SEQ ID NO: 95
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
Heavy chain reference nivolumab analog antibody
SEQ ID NO: 96:
QVQLVESGGGVVQPGRSLRLDCKASGITFSNSGMHWVRQAPGKGLEWVAVIWYDGSKRYYADSVKGRFTISRDNS
KNTLFLQMNSLRAEDTAVYYCATNDDYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGK
Light chain reference nivolumab analog antibody
SEQ ID NO: 97
EIVLTQSPATLSLSPGERATLSCRASQSVSSYLAWYQQKPGQAPRLLIYDASNRATGIPARFSGSGSGTDFTLTI
SSLEPEDFAVYYCQQSSNWPRTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
V region VK3-15
SEQ ID NO: 98
EIVMTQSPATLSVSPGERATLSCRASQSVSSNLAWYQQKPGQAPRLLIYGASTRATGIPA
RFSGSGSGTEFTLTISSLQSEDFAVYYCQQYNNWP
VK3-15/JK1
SEQ ID NO: 99
EIVMTQSPATLSVSPGERATLSCRASQSVSSNLAWYQQKPGQAPRLLIYGASTRATGIPARFSGSGSGTEFTLTI
SSLQSEDFAVYYCQQYNNWPWTFGQGTKVEIK
V region VK3-20
SEQ ID NO: 100
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSP
VK3-20/JK1
SEQ ID NO: 101
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIK
V region VL3-21
SEQ ID NO: 102
SYVLTQPPSVSVAPGETARITCGGDNIGRKSVYWYQQKSGQAPVLVIYYDSDRPSGIPERFSGSNSGNTATLTIS
RVEAGDEADYYCQVWDGSSDH
VL3-21/JL3
SEQ ID NO: 103
SYVLTQPPSVSVAPGETARITCGGDNIGRKSVYWYQQKSGQAPVLVIYYDSDRPSGIPERFSGSNSGNTATLTIS
RVEAGDEADYYCQVWDGSSDHWVFGGGTKLTVL
Extracellular domain TGF-βRII
SEQ ID NO: 104
IPPHVQKSVNNDMIVTDNNGAVKFPQLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNIIFSEEYNTSNPD

1. A multispecific binding moiety comprising a PD-1 binding domain and a TGF-βRII binding domain, wherein the PD-1 binding domain blocks PD-1 mediated signaling and the TGF-βRII binding domain blocks TGF-βRII-mediated signaling.

2. (canceled)

3. The multispecific binding moiety according to claim 1, wherein the multispecific binding moiety comprises a single Fab domain that binds to PD-1, a single Fab domain that binds to TGF-βRII, and an Fc region.

4. The multispecific binding moiety according to claim 1, wherein the multispecific binding moiety has a higher potency in blocking TGF-βRII-mediated signaling in cells expressing both PD-1 and TGF-βRII than in cells expressing TGF-βRII and no, substantially no, or low levels of PD-1.

5. The multispecific binding moiety according to claim 4, wherein the cells expressing both PD-1 and TGF-βRII are Jurkat-PD-1⁺ cells and the cells expressing TGF-βRII and no, or substantially no, PD-1 are Jurkat-PD-1^{null} cells, in particular wherein the potency in blocking TGF-βRII-mediated signaling is measured in a phospho-SMAD2/3 assay.

6. The multispecific binding moiety according to claim 4, wherein the cells expressing both PD-1 and TGF-βRII are activated CD4⁺ and/or CD8⁺ cells and the cells expressing TGF-βRII and no PD-1 are non-activated CD4⁺ and/or CD8⁺ cells, in particular wherein the potency in blocking TGF-βRII-mediated signaling is measured in a phospho-SMAD2/3 assay.

7. The multispecific binding moiety according to claim 4, wherein the cells expressing both PD-1 and TGF-βRII are HEK-Blue TGF-β-PD-1⁺ cells and the cells expressing TGF-βRII and no PD-1 are HEK-Blue TGF-β cells, in particular wherein the potency in blocking TGF-βRII-mediated signaling is measured in an isogenic PD-1-TGF-β reporter assay.

8-9. (canceled)

10. The multispecific binding moiety according to claim 4, wherein the potency in blocking TGF-βRII-mediated signaling in cells expressing both PD-1 and TGF-βRII is at least about 200 fold, preferably between about 200-30000 fold, higher than in cells expressing TGF-βRII and no, substantially no, or low levels of PD-1.

11. The multispecific binding moiety according to claim 4, wherein the potency of the multispecific binding moiety in blocking TGF-βRII-mediated signaling in cells expressing TGF-βRII and no PD-1 is lower than the potency of a reference anti-TGF-βRII antibody and the potency of the multispecific binding moiety in blocking TGF-βRII-mediated signaling in cells expressing both TGF-βRII and PD-1 is higher than the potency of the reference anti-TGF-βRII antibody, wherein the reference anti-TGF-βRII antibody is a bivalent monospecific antibody comprising a heavy chain having an amino acid sequence as set forth in SEQ ID NO: 76 and a light chain having an amino acid sequence as set forth in SEQ ID NO: 77.

12. The multispecific binding moiety according to claim 11, wherein the potency of the multispecific binding moiety in blocking TGF-βRII-mediated signaling in cells expressing both TGF-βRII and PD-1 is at least about 100 fold, preferably between about 100-20000 fold, higher than the potency of the reference anti-TGF-βRII antibody.

13. The multispecific binding moiety according to claim 1, wherein the multispecific binding moiety has a higher activity in reducing tumor volume than a combination of reference antibodies, wherein the combination of reference antibodies are two bivalent monospecific antibodies targeting PD-1 and TGF-βRII, wherein the bivalent monospecific antibody targeting PD-1 comprises a heavy chain having an amino acid sequence as set forth in SEQ ID NO: 78 and a light chain having an amino acid sequence as set forth in SEQ ID NO: 79, and the bivalent monospecific antibody targeting TGF-βRII comprises a heavy chain having an amino acid sequence as set forth in SEQ ID NO: 76 and a light chain having an amino acid sequence as set forth in SEQ ID NO: 77.

14. The multispecific binding moiety according to claim 13, wherein the activity in reducing tumor volume is determined by measuring tumor volume reduction in an in vivo mouse study, in particular in an in vivo mouse study using MDA-MB-231 xenograft huCD34 NSG mice.

15. The multispecific antibody according to claim 13, wherein a higher activity in reducing tumor volume is a tumor volume reduction of at least about 1.5 fold, preferably between about 1.5-100 fold, of the tumor volume reduction of the combination of reference antibodies.

16. A multispecific binding moiety comprising a PD-1 binding domain and a TGF-βRII binding domain, wherein the PD-1 binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;
b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;
c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;
d) heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; or
e) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively;
wherein each of the HCDRs may comprise at most three, two, or one amino acid variations.

17. The multispecific binding moiety according to claim 16, wherein the PD-1 binding domain comprises a heavy chain variable region having an amino acid sequence as set forth in any one of SEQ ID NO: 1; 5; 9; 13; 14; 18; 19, or having at least 80%, preferably 85%, more preferably 90%, or most preferably 95% sequence identity thereto.

18-19. (canceled)

20. The multispecific binding moiety according to claim 16, wherein the TGF-βRII binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;
b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;
d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or
g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,
wherein each of the HCDRs may comprise at most three, two, or one amino acid variations.

21. The multispecific binding moiety according to claim 16, wherein the TGF-βRII binding domain comprises a heavy chain variable region having an amino acid sequence as set forth in any one of SEQ ID NO: 23; 27; 31; 35; 39; 43; 47; 88; 89, or having at least 80%, preferably 85%, more preferably 90%, or most preferably 95% sequence identity thereto.

22. The multispecific binding moiety according to claim 16, wherein the PD-1 binding domain and/or TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, or a variant thereof.

23. The multispecific binding moiety according to claim 16, wherein the PD-1 binding domain and/or TGF-βRII binding domain comprises a light chain variable region having an amino acid sequence as set forth in SEQ ID NO: 48, or having at least 80%, preferably 85%, more preferably 90%, or most preferably 95% sequence identity thereto.

24. A multispecific binding moiety comprising a PD-1 binding domain and a TGF-βRII binding domain, wherein the TGF-βRII binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;
b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;
d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or
g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,
wherein each of the HCDRs may comprise at most three, two, or one amino acid variations.

25. The multispecific binding moiety according to claim 24, wherein the TGF-βRII binding domain comprises a heavy chain variable region having an amino acid sequence as set forth in any one of SEQ ID NO: 23; 27; 31; 35; 39; 43; 47; 88; 89, or having at least 80%, preferably 85%, more preferably 90%, or most preferably 95% sequence identity thereto.

26-27. (canceled)

28. The multispecific binding moiety according to claim 24, wherein the PD-1 binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;
b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;
c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;
d) heavy chain CDR1 (HCDR1), heavy chain CDR2 (HCDR2), and heavy chain CDR3 (HCDR3), having an amino acid sequence as set forth in SEQ ID NO: 15, SEQ ID NO: 16, and SEQ ID NO: 17, respectively; or
e) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively;
wherein each of the HCDRs may comprise at most three, two, or one amino acid variations.

29. The multispecific binding moiety according to claim 28, wherein the PD-1 binding domain comprises a heavy chain variable region having an amino acid sequence as set forth in any one of SEQ ID NO: 1; 5; 9; 13; 14; 18; 19, or having at least 80%, preferably 85%, more preferably 90%, or most preferably 95% sequence identity thereto.

30. The multispecific binding moiety according to claim 28, wherein the PD-1 binding domain and/or TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, or a variant thereof.

31. The multispecific binding moiety according to claim 28, wherein the PD-1 binding domain and/or TGF-βRII binding domain comprises a light chain variable region having an amino acid sequence as set forth in SEQ ID NO: 48, or having at least 80% sequence identity thereto.

32. (canceled)

33. A pharmaceutical composition comprising an effective amount of the multispecific binding moiety according to claim 1, and a pharmaceutically acceptable carrier.

34-35. (canceled)

36. A method for treating a disease, comprising administering an effective amount of a multispecific binding moiety according to claim 1, to a subject in need thereof.

37. A method for treating a disease associated with a suppressed immune system, in particular cancer, comprising administering an effective amount of a multispecific binding moiety according to claim 1, to a subject in need thereof.

38. A cell comprising a nucleic acid sequence encoding the heavy chain variable region of a PD-1 binding domain as defined in claim 16 and a nucleic acid sequence encoding a heavy chain variable region of a TGF-βRII binding domain, wherein the TGF-βRII binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;
b) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
c) heavy chain CDR1 (HCDR1), heavy chain CDR2 (HCDR2), and heavy chain CDR3 (HCDR3), having an amino acid sequence as set forth in SEQ ID NO: 32, SEQ ID NO: 33, and SEQ ID NO: 34, respectively;
d) heavy chain CDR1 (HCDR1), heavy chain CDR2 (HCDR2), and heavy chain CDR3 (HCDR3), having an amino acid sequence as set forth in SEQ ID NO: 36, SEQ ID NO: 37, and SEQ ID NO: 38, respectively;
e) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
f) heavy chain CDR1 (HCDR1), heavy chain CDR2 (HCDR2), and heavy chain CDR3 (HCDR3), having an amino acid sequence as set forth in SEQ ID NO: 44, SEQ ID NO: 45, and SEQ ID NO: 46, respectively; or
g) heavy chain CDR1 (HCDR1), heavy chain CDR2 (HCDR2), and heavy chain CDR3 (HCDR3), having an amino acid sequence as set forth in SEQ ID NO: 90, SEQ ID NO: 91, and SEQ ID NO: 92, respectively,
wherein each of the HCDRs may comprise at most three, two, or one amino acid variations.

39. The cell according to claim 38, wherein the cell further comprises a nucleic acid sequence encoding a CH1 region and preferably a hinge, CH2 and CH3 region.

40. The cell according to claim 38, wherein the cell further comprises at least one nucleic acid sequence encoding a light chain variable region wherein the light chain variable region comprising light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, or a variant thereof, and preferably a CL region.

41. A cell producing a multispecific binding moiety as claimed in claim 1.


SEQUENCE LISTING (110 sequences)

SEQ ID NO: 1 - protein, 120 aa, synthetic construct
  REGION 1..120: note = Heavy chain variable region
EVQLVQSGAEVKKPGSSMKVSCKASGGTFSSYVISWVRQAPGQGLEWMGMIIPVFDTSSYEKKFQGRITIIADKSTSTVYLELSSLRSEDAAVYYCARGTVEATLLFDFWGQGTLVTVSS

SEQ ID NO: 2 - protein, 5 aa, synthetic construct
  REGION 1..5: note = Heavy chain CDR1
SYVIS

SEQ ID NO: 3 - protein, 17 aa, synthetic construct
  REGION 1..17: note = Heavy chain CDR2
MIIPVFDTSSYEKKFQG

SEQ ID NO: 4 - protein, 11 aa, synthetic construct
  REGION 1..11: note = Heavy chain CDR3
GTVEATLLFDF

SEQ ID NO: 5 - protein, 121 aa, synthetic construct
  REGION 1..121: note = Heavy chain variable region
QVQLQESGPGLVKPSETLSLTCTVSNGSLGFDFWSWIRQPPGRGLEWIGYIYYSGSWSLNPSFKGRVTMSVDTSKNQFSLNLRSVTAADTAVYYCARGGYTGYGGDWFDPWGQGTLVTVSS

SEQ ID NO: 6 - protein, 5 aa, synthetic construct
  REGION 1..5: note = Heavy chain CDR1
FDFWS

SEQ ID NO: 7 - protein, 16 aa, synthetic construct
  REGION 1..16: note = Heavy chain CDR2
YIYYSGSWSLNPSFKG

SEQ ID NO: 8 - protein, 13 aa, synthetic construct
  REGION 1..13: note = Heavy chain CDR3
GGYTGYGGDWFDP

SEQ ID NO: 9 - protein, 121 aa, synthetic construct
  REGION 1..121: note = Heavy chain variable region
QVQLQESGPGLVKPSETLSLTCTVSDGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 10 - protein, 5 aa, synthetic construct
  REGION 1..5: note = Heavy chain CDR1
YHFWS

SEQ ID NO: 11 - protein, 16 aa, synthetic construct
  REGION 1..16: note = Heavy chain CDR2
YIVYSGSYNVNPSLKT

SEQ ID NO: 12 - protein, 13 aa, synthetic construct
  REGION 1..13: note = Heavy chain CDR3
GGYTGYGGDWFDP

SEQ ID NO: 13 - protein, 121 aa, synthetic construct
  REGION 1..121: note = Heavy chain variable region
QVQLQESGPGLVKPSETLSLTCTVSEGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 14 - protein, 127 aa, synthetic construct
  REGION 1..127: note = Heavy chain variable region
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSS

SEQ ID NO: 15 - protein, 5 aa, synthetic construct
  REGION 1..5: note = Heavy chain CDR1
RFALH

SEQ ID NO: 16 - protein, 17 aa, synthetic construct
  REGION 1..17: note = Heavy chain CDR2
WIDPNTGTPTFAQGVTG

SEQ ID NO: 17 - protein, 18 aa, synthetic construct
  REGION 1..18: note = Heavy chain CDR3
SLGYCDSDICYPNWIFDN

SEQ ID NO: 18 - protein, 127 aa, synthetic construct; identical to SEQ ID NO: 14
  REGION 1..127: note = Heavy chain variable region

SEQ ID NO: 19 - protein, 127 aa, synthetic construct
  REGION 1..127: note = Heavy chain variable region
QVQLVQSGSELKKPGASVKVSCKASGYTFTRFALSWVRQAPGQGLEWMGWIDPNTGTPTYAQDFTGRFVFSLDTSVTTAYLQISSLKAEDTAVYYCARSLGYCGSDICYPNGILDNWGQGTLVTVSS

SEQ ID NO: 20 - protein, 5 aa, synthetic construct
  REGION 1..5: note = Heavy chain CDR1
RFALS

SEQ ID NO: 21 - protein, 17 aa, synthetic construct
  REGION 1..17: note = Heavy chain CDR2
WIDPNTGTPTYAQDFTG

SEQ ID NO: 22 - protein, 18 aa, synthetic construct
  REGION 1..18: note = Heavy chain CDR3
SLGYCGSDICYPNGILDN

SEQ ID NO: 23 - protein, 122 aa, synthetic construct
  REGION 1..122: note = Heavy chain variable region
EVQLVESGGGLVQPGGSLRLSCAASGFTFDIYAMTWVRQAPGKGLEWVSVISGSGGTTYYADSVKGRFTISRDNSKNTLYLQMNSLRAEDTAVYYCARRGQYRDIVGATDYWGQGTLVTVSS

SEQ ID NO: 24 - protein, 5 aa, synthetic construct
  REGION 1..5: note = Heavy chain CDR1
IYAMT

SEQ ID NO: 25 - protein, 17 aa, synthetic construct
  REGION 1..17: note = Heavy chain CDR2
VISGSGGTTYYADSVKG

SEQ ID NO: 26 - protein, 13 aa, synthetic construct
  REGION 1..13: note = Heavy chain CDR3
RGQYRDIVGATDY

SEQ ID NO: 27 - protein, 122 aa, synthetic construct
  REGION 1..122: note = Heavy chain variable region
EVQLVESGGGLVQPGGSLRLSCAASGFTFDINAMTWVRQAPGKGLEWVSVISGSGGTTYYADSVKGRFTISRDNSKNTLYLQMNSLRAEDTAVYYCARRGQYRDIVGATDYWGQGTLVTVSS

SEQ ID NO: 28 - protein, 5 aa, synthetic construct
  REGION 1..5: note = Heavy chain CDR1
INAMT

SEQ ID NO: 29 - protein, 17 aa, synthetic construct
  REGION 1..17: note = Heavy chain CDR2
VISGSGGTTYYADSVKG

SEQ ID NO: 30 - protein, 13 aa, synthetic construct
  REGION 1..13: note = Heavy chain CDR3
RGQYRDIVGATDY

SEQ ID NO: 31 - protein, 116 aa, synthetic construct
  REGION 1..116: note = Heavy chain variable region
QVQLVESGGGLVEPGGSLRLSCAASGFTFSNAWMSWVRQAPGKGLEWVGRIKTTISGGATDFAAPVKGRFTISRDDSKNTLYLQMNSLKTEDTAVYYCTLDLRDYWGQGTLVTVSS

SEQ ID NO: 32 - protein, 5 aa, synthetic construct
  REGION 1..5: note = Heavy chain CDR1
NAWMS

SEQ ID NO: 33 - protein, 19 aa, synthetic construct
  REGION 1..19: note = Heavy chain CDR2
RIKTTISGGATDFAAPVKG

SEQ ID NO: 34 - protein, 5 aa, synthetic construct
  REGION 1..5: note = Heavy chain CDR3
DLRDY

SEQ ID NO: 35 - protein, 116 aa, synthetic construct
  REGION 1..116: note = Heavy chain variable region
QVQLVESGGGLVEPGGSLRLSCAASGFKFSNAWMSWVRQAPGKGLEWVGRIKTTISGGATQFAAPVKGRFTISRDDSKNTLYLQMNSLKTEDTAVYYCTLDLRDYWGQGTLVTVSS

SEQ ID NO: 36 - protein, 5 aa, synthetic construct
  REGION 1..5: note = Heavy chain CDR1
NAWMS

SEQ ID NO: 37 - protein, 19 aa, synthetic construct
  REGION 1..19: note = Heavy chain CDR2
RIKTTISGGATQFAAPVKG

SEQ ID NO: 38 - protein, 5 aa, synthetic construct
  REGION 1..5: note = Heavy chain CDR3
DLRDY

SEQ ID NO: 39 - protein, 121 aa, synthetic construct
  REGION 1..121: note = Heavy chain variable region
QVQLVESGGGLVQPGGSLRLSCAVSGFTFRRYAMSWVRQAPGKGLEWVSAISASGDRTHNTDSVKGRFSISRDNSKNTLYLQMNSLRAEDTAVYFCAKGIAASGKNYFDPWGQGTLVTVSS

SEQ ID NO: 40 - protein, 5 aa, synthetic construct
  REGION 1..5: note = Heavy chain CDR1
RYAMS

SEQ ID NO: 41 - protein, 17 aa, synthetic construct
  REGION 1..17: note = Heavy chain CDR2
AISASGDRTHNTDSVKG

SEQ ID NO: 42 - protein, 12 aa, synthetic construct
  REGION 1..12: note = Heavy chain CDR3
GIAASGKNYFDP

SEQ ID NO: 43 - protein, 121 aa, synthetic construct
  REGION 1..121: note = Heavy chain variable region
QVQLVESGGGLVQPGGSLRLSCAVSGFTFSRYAMSWVRQAPGKGLEWVSAISASGDRTKNTDSVKGRFSISRDNSKNTLYLQMNSLRAEDTAVYFCAKGTAAAGKNYFDPWGQGTLVTVSS

SEQ ID NO: 44 - protein, 5 aa, synthetic construct
  REGION 1..5: note = Heavy chain CDR1
RYAMS

SEQ ID NO: 45 - protein, 17 aa, synthetic construct
  REGION 1..17: note = Heavy chain CDR2
AISASGDRTKNTDSVKG

SEQ ID NO: 46 - protein, 12 aa, synthetic construct
  REGION 1..12: note = Heavy chain CDR3
GTAAAGKNYFDP

SEQ ID NO: 47 - protein, 121 aa, synthetic construct
  REGION 1..121: note = Heavy chain variable region
QVQLVESGGGLVQPGGSLRLSCAVSGFTFSRYAMSWVRQAPGKGLEWVSAISASGDRTKYTDSVKGRFSISRDNSKNTLYLQMNSLRAEDTAVYFCAKGTAAAGKNYFDPWGQGTLVTVSS

SEQ ID NO: 48 - protein, 107 aa, synthetic construct
  REGION 1..107: note = Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 49 - protein, 6 aa, synthetic construct
  REGION 1..6: note = Light chain CDR1 according to IMGT
QSISSY

SEQ ID NO: 50 - skipped

SEQ ID NO: 51 - protein, 9 aa, synthetic construct
  REGION 1..9: note = Light chain CDR3 according to IMGT
QQSYSTPPT

SEQ ID NO: 52 - protein, 108 aa, synthetic construct
  REGION 1..108: note = Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 53 - protein, 6 aa, synthetic construct
  REGION 1..6: note = Light chain CDR1 according to IMGT
QSISSY

SEQ ID NO: 54 - skipped

SEQ ID NO: 55 - protein, 10 aa, synthetic construct
  REGION 1..10: note = Light chain CDR3 according to IMGT
QQSYSTPPIT

SEQ ID NO: 56 - protein, 107 aa, synthetic construct
  REGION 1..107: note = Light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 57 - protein, 6 aa, synthetic construct
  REGION 1..6: note = Light chain CDR1 according to IMGT
QSVSSN

SEQ ID NO: 58 - skipped

SEQ ID NO: 59 - protein, 9 aa, synthetic construct
  REGION 1..9: note = Light chain CDR3 according to IMGT
QQYNNWPWT

SEQ ID NO: 60 - protein, 108 aa, synthetic construct
  REGION 1..108: note = Light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 61 - protein, 7 aa, synthetic construct
  REGION 1..7: note = Light chain CDR1 according to IMGT
QSVSSSY

SEQ ID NO: 62 - skipped

SEQ ID NO: 63 - protein, 9 aa, synthetic construct
  REGION 1..9: note = Light chain CDR3 according to IMGT
QQYGSSPWT

SEQ ID NO: 64 - protein, 108 aa, synthetic construct
  REGION 1..108: note = Light chain variable region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 65 - protein, 6 aa, synthetic construct
  REGION 1..6: note = Light chain CDR1 according to IMGT
NIGRKS

SEQ ID NO: 66 - skipped

SEQ ID NO: 67 - protein, 11 aa, synthetic construct
  REGION 1..11: note = Light chain CDR3 according to IMGT
QVWDGSSDHWV

SEQ ID NO: 68 - protein, 15 aa, synthetic construct
  REGION 1..15: note = hinge region
EPKSCDKTHTCPPCP

SEQ ID NO: 69 - protein, 98 aa, synthetic construct
  REGION 1..98: note = CH1 region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 70 - protein, 110 aa, synthetic construct
  REGION 1..110: note = CH2 region
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 71 - protein, 110 aa, synthetic construct
  REGION 1..110: note = CH2-DM region
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 72 - protein, 106 aa, synthetic construct
  REGION 1..106: note = CH3 region
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 106 aa, synthetic construct
  REGION 1..106: note = CH3-DE region
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 106 aa, synthetic construct
  REGION 1..106: note = CH3-KK region
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 107 aa, synthetic construct [sequence omitted]

SEQ ID NO: 76 - protein, 451 aa, synthetic construct
  REGION 1..451: note = Heavy chain reference anti-TGF-betaRII antibody TGF1 analog
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, synthetic construct
  REGION 1..214: note = Light chain reference anti-TGF-betaRII antibody TGF1 analog
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 447 aa, synthetic construct
  REGION 1..447: note = Heavy chain reference PD-1 antibody pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 218 aa, synthetic construct
  REGION 1..218: note = Light chain reference PD-1 antibody pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 607 aa, synthetic construct
  REGION 1..607: note = Heavy chain reference PD-L1-TGF-beta TRAP molecule analog
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 81 - protein, 216 aa, synthetic construct
  REGION 1..216: note = Light chain reference PD-L1-TGF-beta TRAP molecule analog
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 82 - protein, 567 aa, synthetic construct
  REGION 1..567: note = human TGF-betaRII isoform A
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 83 - protein, 144 aa, synthetic construct
  REGION 1..144: note = extracellular domain of human TGF-betaRII isoform A
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 84 - protein, 592 aa, synthetic construct
  REGION 1..592: note = human TGF-betaRII isoform B
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 85 - protein, 169 aa, synthetic construct
  REGION 1..169: note = extracellular domain of isoform B of human TGF-betaRII
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 86 - protein, 454 aa, synthetic construct
  REGION 1..454: note = Heavy chain negative control RSV IgG1 antibody
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 214 aa, synthetic construct
  REGION 1..214: note = Light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 121 aa, synthetic construct
  REGION 1..121: note = Heavy chain variable region
QVQLVESGGGLVQPGGSLRLSCAVSGFTFSRYAMSWVRQAPGKGLEWVSAISASGDRTKNTDSVKGRFSISRDNSKNTLYLQMNSLRAEDTAVYYCAKGTAAAGKNYFDPWGQGTLVTVSS

SEQ ID NO: 89 - protein, 121 aa, synthetic construct
  REGION 1..121: note = Heavy chain variable region
QVQLVESGGGLVQPGGSLRLSCAVSGFTFSRYAMSWVRQAPGKGLEWVSAISASGDRTKYTDSVKGRFSISRDNSKNTLYLQMNSLRAEDTAVYYCAKGTAAAGKNYFDPWGQGTLVTVSS

SEQ ID NO: 90 - protein, 5 aa, synthetic construct
  REGION 1..5: note = Heavy chain CDR1
RYAMS

SEQ ID NO: 91 - protein, 17 aa, synthetic construct
  REGION 1..17: note = Heavy chain CDR2
AISASGDRTKYTDSVKG

SEQ ID NO: 92 - protein, 12 aa, synthetic construct
  REGION 1..12: note = Heavy chain CDR3
GTAAAGKNYFDP

SEQ ID NO: 93 - protein, 95 aa, synthetic construct
  REGION 1..95: note = V region VK1-39
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 94 - protein, 107 aa, synthetic construct; identical to SEQ ID NO: 48
  REGION 1..107: note = VK1-39/JK1

SEQ ID NO: 95 - protein, 108 aa, synthetic construct; identical to SEQ ID NO: 52
  REGION 1..108: note = VK1-39/JK5

SEQ ID NO: 96 - protein, 440 aa, synthetic construct
  REGION 1..440: note = Heavy chain reference nivolumab analog antibody
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 214 aa, synthetic construct
  REGION 1..214: note = Light chain reference nivolumab analog antibody
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 95 aa, synthetic construct
  REGION 1..95: note = V region VK3-15
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 99 - protein, 107 aa, synthetic construct; identical to SEQ ID NO: 56
  REGION 1..107: note = VK3-15/JK1

SEQ ID NO: 100 - protein, 96 aa, synthetic construct
  REGION 1..96: note = V region VK3-20
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 101 - protein, 108 aa, synthetic construct; identical to SEQ ID NO: 60
  REGION 1..108: note = VK3-20/JK1

SEQ ID NO: 102 - protein, 96 aa, synthetic construct
  REGION 1..96: note = V region VL3-21
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 103 - protein, 108 aa, synthetic construct; identical to SEQ ID NO: 64
  REGION 1..108: note = VL3-21/JL3

SEQ ID NO: 104 - protein, 136 aa, synthetic construct
  REGION 1..136: note = Extracellular domain TGF-betaRII
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 105 - skipped

SEQ ID NO: 106 - protein, 16 aa, synthetic construct
  REGION 1..16: note = HCDR2
  VARIANT 3: note = X can be Y, V, or I
  VARIANT 7: note = X can be S, or G
  VARIANT 8: note = X can be T, Y, S, H, N, W, L, or Q
  VARIANT 9: note = X can be S, or N
  VARIANT 10: note = X can be F, V, or L
  VARIANT 11: note = X can be N, or S
  VARIANT 13: note = X can be S or A
  VARIANT 14: note = X can be F or L
  VARIANT 16: note = X can be S, T, G, D, R, or N
YIXYSGXXXXXPXXKX

SEQ ID NO: 107 - protein, 13 aa, synthetic construct
  REGION 1..13: note = HCDR3
  VARIANT 6: note = X can be Y, H, V, or A
  VARIANT 13: note = X can be P, V, Y, W, F, T, Q, H, or S
GGYTGXGGDWFDX

SEQ ID NO: 108 - skipped

SEQ ID NO: 109 - protein, 17 aa, synthetic construct
  REGION 1..17: note = HCDR2
  VARIANT 4: note = X can be P, S, or T
  VARIANT 5: note = X can be N, or Q
  VARIANT 6: note = X can be T, or D
  VARIANT 8: note = X can be N, S, T, K, L, or E
  VARIANT 9: note = X can be P, Y, A, H, or F
  VARIANT 10: note = X can be T, or S
  VARIANT 11: note = X can be Y, F, or H
  VARIANT 12: note = X can be A, G, V, or F
  VARIANT 13: note = X can be Q, R, N, L, T, or S
  VARIANT 14: note = X can be D, A, G, or S
  VARIANT 15: note = X can be F, V, or A
  VARIANT 16: note = X can be T, K, H, G
  VARIANT 17: note = X can be G, N, E, or D
WIBXXXGXXXXXXXXXX

SEQ ID NO: 110 - protein, 18 aa, synthetic construct
  REGION 1..18: note = HCDR3
  VARIANT 1: note = X can be I, S, or V
  VARIANT 2: note = X can be L, Q, or N
  VARIANT 6: note = X can be N, G, S, or D
  VARIANT 7: note = X can be T, S, P, N, or E
  VARIANT 9: note = X can be N, or I
  VARIANT 14: note = X can be W, G, Q, H, W, A, or L
  VARIANT 15: note = X can be I, V, or L
  VARIANT 16: note = X can be F, L, or I
  VARIANT 18: note = X can be Y, S, N, I, R, H, V, T, K, A, or L
XXGYCXXDXCYPNXXXDX